(19) United States
(12) Patent Application Publication
(10) Pub. No.: US 2025/0295767 A1
(43) Pub. Date: Sep. 25, 2025

(54) ROR1-SPECIFIC ANTIGEN BINDING MOLECULES

(71) Applicant: ALMAC DISCOVERY LIMITED, Craigavon (GB)

(72) Inventors: Estelle Grace McLean, Craigavon (GB); Paul Richard Trumper, Craigavon (GB); Jennifer Thom, Craigavon (GB); Timothy Harrison, Craigavon (GB); Graham John Cotton, Craigavon (GB); Caroline Barelle, Craigavon (GB); Andrew Porter, Craigavon (GB); Marina Kovaleva, Craigavon (GB)

(73) Assignee: ALMAC DISCOVERY LIMITED, Craigavon (GB)

(21) Appl. No.: 19/230,346

(22) Filed: Jun. 6, 2025

Related U.S. Application Data
(63) Continuation of application No. 16/957,075, filed on Jun. 22, 2020, now Pat. No. 12,324,837.

(30) Foreign Application Priority Data
Dec. 22, 2017 (GB) 1721802.5

(51) Int. Cl. A61K 39/395; A61K 39/00; A61K 40/11; A61K 40/31; A61K 40/42; A61K 47/68; A61P 35/00; C07K 16/28; C07K 16/46; G01N 33/574

(52) U.S. Cl. CPC A61K 39/39558; A61K 40/11; A61K 40/31; A61K 40/4202; A61K 47/68031; A61K 47/68035; A61K 47/6817; A61K 47/6849; A61K 47/6851; A61P 35/00; C07K 16/2803; C07K 16/2863; C07K 16/46; G01N 33/57492; A61K 2039/5156; C07K 2317/24; C07K 2317/31; C07K 2317/33; C07K 2317/56; C07K 2317/565; C07K 2317/567; C07K 2317/73; C07K 2317/76; C07K 2317/77; C07K 2317/92; C07K 2317/94; C07K 2319/03; C07K 2319/30; C07K 2319/33; G01N 2333/70503; G01N 2333/71

(57) ABSTRACT

The present invention relates to receptor tyrosine kinase-like orphan receptor I (RORI) specific antigen binding molecules and associated fusion proteins and conjugates. In a further aspect, the present invention relates to coajugated immunoglobulin-like shark variable novel antigen receptors (VNARs).

### RELATED APPLICATIONS

The present application is a continuation of U.S. application Ser. No. 16/957,075 filed on Jun. 22, 2020, which is a US National Stage application of PCT/EP2018/086823 filed on Dec. 21, 2018, which claims priority from GB Patent Application No. 1721802.5, filed Dec. 22, 2017, the entirety of which are incorporated herein by reference.

### SEQUENCE LISTING

The instant application contains a Sequence Listing which has been submitted electronically in XML file format and is hereby incorporated by reference in its entirety. Said XML copy, created on May 15, 2025, is named P114734WO.xml and is 340,881 bytes in size.

### FIELD OF INVENTION

The present invention relates to receptor tyrosine kinase-like orphan receptor 1 (ROR1) specific antigen binding molecules and associated fusion proteins and conjugates. In a further aspect, the present invention relates to conjugated immunoglobulin-like shark variable novel antigen receptors (VNARs).

### BACKGROUND

Receptor tyrosine kinase-like orphan receptor 1 (ROR1) is a 937 amino acid glycosylated type I single pass transmembrane protein. The extracellular region consists of three distinct domains composing an N-terminal immunoglobulin domain (Ig), followed by a cysteine rich fizzled domain (fz) which in turn is linked to the membrane proximal kringle domain (kr). The intracellular region of the protein contains a pseudo kinase domain followed by two Ser/Thr rich domains which are interspersed by a proline-rich region, and this same overall domain architecture is conserved in the closely related family member ROR2, with which it shares high sequence identity. (Rebagay G et al, Frontiers Oncology, 2012, 2, Borcherding N et al Protein Cell, 2014, 5, 496-502).

ROR1 is expressed during embryonic development, where it is prominently expressed in neural crest cells and in the necrotic and interdigital zones in the later stages of development. However, its expression is quickly silenced after birth, and is largely absent in normal adult tissue (Fukada PNAS, 2012, Baskar et al Clin. Cancer Res., 2008, 14, 396, Broome H E et al, Leuk. Res., 2011, 35, 1390; Balakrishnan A et al, Clin. Cancer. Res. 2017, 23, 3061-3071).

ROR1 expression has been observed at both the mRNA and protein level across a broad range of solid tumours and haematological malignancies including lung, breast, pancreatic, ovarian, colon, head and neck and prostate cancers, melanoma and renal cell carcinoma (Zhang S et al Am J. Pathol., 2012, 181, 1903-1910), breast cancer (Zhang S et al PLoS One 2012, 7, e31127; Oxford Biotherapeutics patent application WO2011054007) and Chronic lymphocytic leukemia (CLL) and acute lymphoblastic leukemia AML (Fukuda T et al, Proc Natl Acad Sci USA. 2008, 105, 3047-3052; Baskar S et al, Clin Cancer Res., 2008, 14, 396-404; Daneshmanesh A H et al, Int J Cancer. 2008, 123, 1190-1195; Dave H et al, PLOS ONE, 2012, 7, e52655).

Additionally, increased ROR1 expression is reported to correlate with poor clinical outcomes for a number of cancer indications including breast cancer (Chien H P et al, Virchows Arch., 2016, 468, 589-595; Zhang), ovarian cancer (Zhang H et al, Sci Rep., 2014, 4:5811. doi: 10.1038/srep05811), colorectal cancer (Zhou J K et al, Oncotarget, 2017, 8, 32864-32872), lung adenocarcinoma (Zheng Y Z et al, Sci Rep., 2016, 6, 36447) and CLL (Cui B et al, Blood, 2016, 128, 2931-2940).

Consistent with ROR1's expression pattern and the link to poor clinical prognosis, a functional role for ROR1 in tumourigenesis and disease progression has been demonstrated for a number of different cancer indications. ROR1 promotes epithelial-mesenchymal transition and metastasis in models of breast cancer (Cui B et al Cancer Res, 2013, 73, 3649-3660) and spheroid formation and tumour engraftment in models of ovarian cancer (Zhang S et al, Proc Natl Acad Sci., 2014, 11, 17266-17271). ROR1 is a transcript target of the NKX2-1/TTF-1 lineage survival factor oncogene in lung adenocarcinoma, where it sustains EGFR signalling and represses pro-apoptotic signalling (Yamaguchi T et al, Cancer Cell, 2012, 21, 348-361; Ida L et al, Cancer Science, 2016, 107, 155-161). ROR1 has also been shown to act as a scaffold to sustain caveolae structures and by-pass signalling mechanism that confer resistance to EGFR tyrosine kinase inhibitors (Yamaguchi T et al, Nat Commun., 2016, 7, 10060). Signalling through an ROR1-HER3 complex modulates the Hippo-YAP pathway and promotes breast cancer bone metastasis (Li C et al, Nature Cell Biol., 19, 1206-119) and the protein can promote Met-driven tumourigenesis (Gentile A et al, Cancer Res., 2011, 71, 3132-3140). Whilst in CLL, ROR1 has been reported to hetero-oligomerise with ROR2 in response to Wnt5a to transduce signalling and enhance proliferation and migration (Yu J et al, J. Clin. Invest., 2016, 2, 585-598)

Given the functional role of ROR1 in cancer pathology and the general lack of expression on normal adult tissue, this oncofetal protein is an attractive target for cancer therapy. Antibodies to ROR1 have been described in the literature WO2021097313 (4A5 kipps), WO2014031174 (UC961), WO2016187220 (Five Prime) WO2010124188 (2A2), WO2012075158 (R11, R12), WO2011054007 (Oxford Bio), WO2011079902 (Bioinvent) WO2017127664, WO2017127664 (NBE Therapeutics, SCRIPPS), WO2016094847 (Emergent), WO2017127499), and a humanised murine anti-ROR1 antibody, UC961, has entered clinical trials for relapsed or refractory chronic lymphocytic leukemia. Chimeric antigen receptor T-cells targeting ROR1 have also been reported (Hudecek M et al, Clin. Cancer Res., 2013, 19, 3153-64) and preclinical primate studies with UC961 and with CAR-T cells targeting ROR1 showed no overt toxicity, which is consistent with the general lack of expression of the protein on adult tissue (Choi M et al, Clinical Lymphoma, myeloma & leukemia, 2015, S167; Berger C et al, Cancer Immunol. Res., 2015, 3, 206).

Single domain binding molecules can be derived from an array of proteins from distinct species. The immunoglobulin isotope novel antigen receptor (IgNAR) is a homodimeric heavy-chain complex originally found in the serum of the nurse shark (Ginglymostoma cirratum) and other sharks and ray species. IgNARs do not contain light chains and are distinct from the typical immunoglobulin structure. Each molecule consists of a single-variable domain (VNAR) and five constant domains (CNAR). The nomenclature in the literature refers to IgNARs as immunoglobulin isotope novel antigen receptors or immunoglobulin isotope new antigen receptors and the terms are synonymous.

There are three main defined types of shark IgNAR known as I, II and III (Kovalena et al, Exp Opin Biol Ther 2014 14(10) 1527-1539). These have been categorized based on the position of non-canonical cysteine residues which are under strong selective pressure and are therefore rarely replaced.

All three types have the classical immunoglobulin canonical cysteines at positions 35 and 107 that stabilize the standard immunoglobulin fold, together with an invariant tryptophan at position 36. There is no defined CDR2 as such, but regions of sequence variation that compare more closely to TCR HV2 and HV4 have been defined in framework 2 and 3 respectively. Type I has germline encoded cysteine residues in framework 2 and framework 4 and an even number of additional cysteines within CDR3. Crystal structure studies of a Type I IgNAR isolated against and in complex with lysozyme enabled the contribution of these cysteine residues to be determined. Both the framework 2 and 4 cysteines form disulphide bridges with those in CDR3 forming a tightly packed structure within which the CDR3 loop is held tightly down towards the HV2 region. To date Type I IgNARs have only been identified in nurse sharks—all other elasmobranchs, including members of the same order have only Type II or variations of this type.

Type II IgNAR are defined as having a cysteine residue in CDR1 and CDR3 which form intra-molecular disulphide bonds that hold these two regions in close proximity, resulting in a protruding CDR3 that is conducive to binding pockets or grooves. Type I sequences typically have longer CDR3s than type II with an average of 21 and 15 residues respectively. This is believed to be due to a strong selective pressure for two or more cysteine residues in Type I CDR3 to associate with their framework 2 and 4 counterparts. Studies into the accumulation of somatic mutations show that there are a greater number of mutations in CDR1 of type II than type I, whereas HV2 regions of Type I show greater sequence variation than Type II. This evidence correlates well with the determined positioning of these regions within the antigen binding sites. A third IgNAR type known as Type III has been identified in neonates. This member of the IgNAR family lacks diversity within CDR3 due to the germline fusion of the D1 and D2 regions (which form CDR3) with the V-gene. Almost all known clones have a CDR3 length of 15 residues with little or no sequence diversity.

Another structural type of VNAR, termed type (IIb or IV), has only two canonical cysteine residues (in framework 1 and framework 3b regions). So far, this type has been found primarily in dogfish sharks (Liu, J. L., et al. Mol. Immunol. 2007. 44(7): p. 1775-1783; Kovalenko O. V., et al. J Biol Chem. 2013. 288(24): p. 17408-19) and was also isolated from semisynthetic V-NAR libraries derived from wobbegong sharks (Streltsov, V. A. et al. (2004) Proc. Natl. Acad. Sci. U.S.A. 101(34): p. 12444-12449).

### SUMMARY OF INVENTION

The present invention generally relates to specific antigen binding molecules. In a first aspect, there is provided a receptor tyrosine kinase-like orphan receptor 1 (ROR1) specific antigen binding molecule comprising an amino acid sequence represented by the formula (I):

FW1-CDR1-FW2-HV2-FW3a-HV4-FW3b-CDR3-FW4 (I)
wherein
   FW1 is a framework region
   CDR1 is a CDR sequence
   FW2 is a framework region
   HV2 is a hypervariable sequence
   FW3a is a framework region
   HV4 is a hypervariable sequence
   FW3b is a framework region
   CDR3 is a CDR sequence
   FW4 is a framework region.

Framework region FW1 is preferably from 20 to 28 amino acids in length, more preferably from 22 to 26 amino acids in length, still more preferably from 23 to 25 amino acids in length. In certain preferred embodiments, FW1 is 26 amino acids in length. In other preferred embodiments, FW1 is 25 amino acids in length. In still other preferred embodiments, FW1 is 24 amino acids in length.

CDR region CDR1 is preferably from 7 to 11 amino acids in length, more preferably from 8 to 10 amino acids in length. In certain preferred embodiments, CDR1 is 9 amino acids in length. In other preferred embodiments, CDR1 is 8 amino acids in length.

Framework region FW2 is preferably from 6 to 14 amino acids in length, more preferably from 8 to 12 amino acids in length. In certain preferred embodiments, FW2 is 12 amino acids in length. In other preferred embodiments, FW2 is 10 amino acids in length. In other preferred embodiments, FW2 is 9 amino acids in length. In other preferred embodiments, FW2 is 8 amino acids in length.

Hypervariable sequence HV2 is preferably from 4 to 11 amino acids in length, more preferably from 5 to 10 amino acids in length. In certain preferred embodiments, HV2 is 10 amino acids in length. In certain preferred embodiments, HV2 is 9 amino acids in length. In other preferred embodiments, HV2 is 6 amino acids in length.

Framework region FW3a is preferably from 6 to 10 amino acids in length, more preferably from 7 to 9 amino acids in length. In certain preferred embodiments, FW3a is 8 amino acids in length. In certain preferred embodiments, FW3a is 7 amino acids in length.

Hypervariable sequence HV4 is preferably from 3 to 7 amino acids in length, more preferably from 4 to 6 amino acids in length. In certain preferred embodiments, HV4 is 5 amino acids in length. In other preferred embodiments, HV4 is 4 amino acids in length.

Framework region FW3b is preferably from 17 to 24 amino acids in length, more preferably from 18 to 23 amino acids in length, still more preferably from 19 to 22 amino acids in length. In certain preferred embodiments, FW3b is 21 amino acids in length. In other preferred embodiments, FW3b is 20 amino acids in length.

CDR region CDR3 is preferably from 8 to 21 amino acids in length, more preferably from 9 to 20 amino acids in length, still more preferably from 10 to 19 amino acids in length. In certain preferred embodiments, CDR3 is 17 amino acids in length. In other preferred embodiments, CDR3 is 14 amino acids in length. In still other preferred embodiments, CDR3 is 12 amino acids in length. In yet other preferred embodiments, CDR3 is 10 amino acids in length.

Framework region FW4 is preferably from 7 to 14 amino acids in length, more preferably from 8 to 13 amino acids in length, still more preferably from 9 to 12 amino acids in length. In certain preferred embodiments, FW4 is 12 amino acids in length. In other preferred embodiments, FW4 is 11 amino acids in length. In still other preferred embodiments, FW4 is 10 amino acids in length. In yet other preferred embodiments, FW4 is 9 amino acids in length.

Preferably, the ROR1-specific antigen binding molecule does not bind to receptor tyrosine kinase-like orphan receptor 2 (ROR2). More preferably, the ROR1-specific antigen binding molecule binds to both human ROR1 and murine ROR1 (mROR1). Yet more preferably, the ROR1-specific antigen binding molecule binds to deglycosylated ROR1.

Certain ROR1-specific antigen binding molecules of the invention do not bind to a linear peptide sequence selected from:

In preferred embodiments of the ROR1-specific antigen binding molecule:

(SEQ ID NO: 34)
YMESLHMQGEIENQI
(SEQ ID NO: 35)
CQPWNSQYPHTHTFTALRFP
(SEQ ID NO: 36)
RSTIYGSRLRIRNLDTTDTGYFQ
(SEQ ID NO: 37)
QCVATNGKEVVSSTGVLFVKFGPPPTASPGYSDEYE

FW1 is a framework region of from 20 to 28 amino acids
CDR1 is a CDR sequence selected from DTSYGLYS (SEQ ID NO: 1), GAKYGLAA (SEQ ID NO: 2), GAKYGLFA (SEQ ID NO: 3), GANYGLAA (SEQ ID NO: 4), or GANYGLAS (SEQ ID NO: 5)
FW2 is a framework region of from 6 to 14 amino acids
HV2 is a hypervariable sequence selected TTDWERMSIG (SEQ ID NO: 6), SSNQERISIS (SEQ ID NO: 7), or SSNKEQISIS (SEQ ID NO: 8)
FW3a is a framework region of from 6 to 10 amino acids
HV4 is a hypervariable sequence selected from NKRAK (SEQ ID NO: 9), NKRTM (SEQ ID NO: 10), NKGAK (SEQ ID NO: 11), or NKGTK (SEQ ID NO: 12)
FW3b is a framework region of from 17 to 24 amino acids

CDR3 is a CDR sequence selected from
(SEQ ID NO: 13)
QSGMAISTGSGHGYNWY,
(SEQ ID NO: 14)
QSGMAIDIGSGHGYNWY,
(SEQ ID NO: 15)
YPWAMWGQWY,
(SEQ ID NO: 16)
VFMPQHWHPAAHWY,
(SEQ ID NO: 17)
REARHPWLRQWY,
or
(SEQ ID NO: 18)
YPWGAGAPWLVQWY

FW4 is a framework region of from 7 to 14 amino acids
   or a functional variant with at least 45% sequence identity thereto.

In other preferred embodiments of the ROR1-specific antigen binding molecule, FW1 is selected from: ASVNQTPRTATKETGESLTINCVLT (SEQ ID NO: 19), AKVDQTPRTATKETGESLTINCVLT (SEQ ID NO: 20), TRVDQTPRTATKETGESLTINCVVT (SEQ ID NO: 21), TRVDQTPRTATKETGESLTINCVLT (SEQ ID NO: 22), ASVNQTPRTATKETGESLTINCVVT (SEQ ID NO: 23), or TRVDQSPSSLSASVGDRVTITCVLT (SEQ ID NO: 24), FW2 is selected from: TSWFRKNPG (SEQ ID NO: 25), or TYWYRKNPG (SEQ ID NO: 26), FW3a is selected from: GRYVESV (SEQ ID NO: 27), or GRYSESV (SEQ ID NO: 28), FW3b is selected from: SFSLRIKDLTVADSATYYCKA (SEQ ID NO: 29), SFTLTISSLQPEDSATYYCRA (SEQ ID NO: 30), or SFTLTISSLQPEDFATYYCKA (SEQ ID NO: 31), and FW4 is selected from DGAGTVLTVN (SEQ ID NO: 32), or DGAGTKVEIK (SEQ ID NO: 33), or functional variants thereof with a sequence identity of at least 45%.

All possible combinations and permutations of the framework regions, complementarity determining regions and hypervariable regions listed above are explicitly contemplated herein.

Sequence identity referenced in relation to the molecules of the invention may be judged at the level of individual CDRs, HVs or FWs, or it may be judged over the length of the entire molecule. The CDR, HV and FW sequences described may also be longer or shorter, whether that be by addition or deletion of amino acids at the N- or C-terminal ends of the sequence or by insertion or deletion of amino acids with a sequence.

In a preferred embodiment of the ROR1-specific antigen binding molecule, FW1 is ASVNQTPRTATKETGESLTINCVLT (SEQ ID NO: 19); CDR1 is DTSYGLYS (SEQ ID NO: 1); FW2 is TSWFRKNPG (SEQ ID NO: 25); HV2 is TTDWERMSIG (SEQ ID NO: 6); FW3a is GRYVESV (SEQ ID NO: 27); HV4 is NKRAK (SEQ ID NO: 9); FW3b is SFSLRIKDLTVADSATYYCKA (SEQ ID NO: 29); CDR3 is QSGMAISTGSGHGYNWY (SEQ ID NO: 13); and FW4 is DGAGTVLTVN (SEQ ID NO: 32); or functional variants thereof with a sequence identity of at least 45%.

In another preferred embodiment of the ROR1-specific antigen binding molecule, FW1 is AKVDQTPRTATKETGESLTINCVLT (SEQ ID NO: 20); CDR1 is DTSYGLYS (SEQ ID NO: 1); FW2 is TSWFRKNPG (SEQ ID NO: 25); HV2 is TTDWERMSIG (SEQ ID NO: 6); FW3a is GRYVESV (SEQ ID NO: 27); HV4 is NKRAK (SEQ ID NO: 9); FW3b is SFSLRIKDLTVADSATYYCKA (SEQ ID NO: 29); CDR3 is QSGMAIDIGSGHGYNWY (SEQ ID NO: 14); and FW4 is DGAGTVLTVN (SEQ ID NO: 32); or functional variants thereof with a sequence identity of at least 45%.

In another preferred embodiment of the ROR1-specific antigen binding molecule, FW1 is TRVDQTPRTATKETGESLTINCVVT (SEQ ID NO: 21); CDR1 is GAKYGLAA (SEQ ID NO: 2); FW2 is TYWYRKNPG (SEQ ID NO: 26); HV2 is SSNQERISIS (SEQ ID NO: 7); FW3a is GRYVESV (SEQ ID NO: 27); HV4 is NKRTM (SEQ ID NO: 10); FW3b is SFSLRIKDLTVADSATYYCKA (SEQ ID NO: 29); CDR3 is YPWAMWGQWY (SEQ ID NO: 15); and FW4 is DGAGTVLTVN (SEQ ID NO: 32); or functional variants thereof with a sequence identity of at least 45%.

In another preferred embodiment of the ROR1-specific antigen binding molecule, FW1 is TRVDQTPRTATKETGESLTINCVVT (SEQ ID NO: 21); CDR1 is GAKYGLFA (SEQ ID NO: 3); FW2 is TYWYRKNPG (SEQ ID NO: 26); HV2 is SSNQERISIS (SEQ ID NO: 7); FW3a is GRYVESV (SEQ ID NO: 27); HV4 is NKRTM (SEQ ID NO: 10); FW3b is SFSLRIKDLTVADSATYYCKA (SEQ ID NO: 29); CDR3 is VFMPQHWHPAAHWY (SEQ ID NO: 16); and FW4 is DGAGTVLTVN (SEQ ID NO: 32); or functional variants thereof with a sequence identity of at least 45%.

In another preferred embodiment of the ROR1-specific antigen binding molecule, FW1 is TRVDQTPRTATKETGESLTINCVLT (SEQ ID NO: 22); CDR1 is DTSYGLYS (SEQ ID NO: 1); FW2 is TSWFRKNPG (SEQ ID NO: 25); HV2 is TTDWERMSIG (SEQ ID NO: 6); FW3a is GRYVESV (SEQ ID NO: 27); HV4 is NKGAK (SEQ ID NO: 11); FW3b is SFSLRIKDLTVADSATYYCKA (SEQ ID NO: 29); CDR3 is REARHPWLRQWY (SEQ ID NO: 17); and FW4 is DGAGTVLTVN (SEQ ID NO: 32); or functional variants thereof with a sequence identity of at least 45%.

In another preferred embodiment of the ROR1-specific antigen binding molecule, FW1 is ASVNQTPRTATKETGESLTINCVVT (SEQ ID NO: 23); CDR1 is GANYGLAA (SEQ ID NO: 4); FW2 is TYWYRKNPG (SEQ ID NO: 26); HV2 is SSNQERISIS (SEQ ID NO: 7); FW3a is GRYVESV (SEQ ID NO: 27); HV4 is NKRTM (SEQ ID NO: 10); FW3b is SFSLRIKDLTVADSATYYCKA (SEQ ID NO: 29); CDR3 is YPWGAGAPWLVQWY (SEQ ID NO: 18); and FW4 is DGAGTVLTVN (SEQ ID NO: 32); or functional variants thereof with a sequence identity of at least 45%.

In another preferred embodiment of the ROR1-specific antigen binding molecule, FW1 is TRVDQSPSSLSASVGDRVTITCVLT (SEQ ID NO: 24); CDR1 is GANYGLAS (SEQ ID NO: 5); FW2 is TYWYRKNPG (SEQ ID NO: 26); HV2 is SSNKEQISIS (SEQ ID NO: 8); FW3a is GRYSESV (SEQ ID NO: 28); HV4 is NKGTK (SEQ ID NO: 12); FW3b is SFTLTISSLQPEDSATYYCRA (SEQ ID NO: 30); CDR3 is YPWGAGAPWLVQWY (SEQ ID NO: 18); and FW4 is DGAGTKVEIK (SEQ ID NO: 33); or functional variants thereof with a sequence identity of at least 45%.

In another preferred embodiment of the ROR1-specific antigen binding molecule, FW1 is TRVDQSPSSLSASVGDRVTITCVLT (SEQ ID NO: 24); CDR1 is GANYGLAS (SEQ ID NO: 5); FW2 is TYWYRKNPG (SEQ ID NO: 26); HV2 is SSNQERISIS (SEQ ID NO: 7); FW3a is GRYSESV (SEQ ID NO: 28); HV4 is NKRTM (SEQ ID NO: 10); FW3b is SFTLTISSLQPEDSATYYCRA (SEQ ID NO: 30); CDR3 is YPWGAGAPWLVQWY (SEQ ID NO: 18); and FW4 is DGAGTKVEIK (SEQ ID NO: 33); or functional variants thereof with a sequence identity of at least 45%.

In another preferred embodiment of the ROR1-specific antigen binding molecule, FW1 is TRVDQSPSSLSASVGDRVTITCVLT (SEQ ID NO: 24); CDR1 is DTSYGLYS (SEQ ID NO: 1); FW2 is TSWFRKNPG (SEQ ID NO: 25); HV2 is TTDWERMSIG (SEQ ID NO: 6); FW3a is GRYVESV (SEQ ID NO: 27); HV4 is NKGAK (SEQ ID NO: 11); FW3b is SFTLTISSLQPEDFATYYCKA (SEQ ID NO: 31); CDR3 is REARHPWLRQWY (SEQ ID NO: 17); and FW4 is DGAGTKVEIK (SEQ ID NO: 33); or functional variants thereof with a sequence identity of at least 45%.

In another preferred embodiment of the ROR1-specific antigen binding molecule, FW1 is TRVDQSPSSLSASVGDRVTITCVLT (SEQ ID NO: 24); CDR1 is DTSYGLYS (SEQ ID NO: 1); FW2 is TYWYRKNPG (SEQ ID NO: 26); HV2 is SSNKEQISIS (SEQ ID NO: 8); FW3a is GRYSESV (SEQ ID NO: 28); HV4 is NKGTK (SEQ ID NO: 12); FW3b is SFTLTISSLQPEDSATYYCRA (SEQ ID NO: 30); CDR3 is REARHPWLRQWY (SEQ ID NO: 17); and FW4 is DGAGTKVEIK (SEQ ID NO: 33); or functional variants thereof with a sequence identity of at least 45%.

In another preferred embodiment of the ROR1-specific antigen binding molecule, FW1 is TRVDQSPSSLSASVGDRVTITCVLT (SEQ ID NO: 24); CDR1 is DTSYGLYS (SEQ ID NO: 1); FW2 is TYWYRKNPG (SEQ ID NO: 26); HV2 is TTDWERMSIG (SEQ ID NO: 6); FW3a is GRYSESV (SEQ ID NO: 28); HV4 is NKGAK (SEQ ID NO: 11); FW3b is SFTLTISSLQPEDSATYYCRA (SEQ ID NO: 30); CDR3 is REARHPWLRQWY (SEQ ID NO: 17); and FW4 is DGAGTKVEIK (SEQ ID NO: 33); or functional variants thereof with a sequence identity of at least 45%.

In yet further preferred embodiments, the ROR1-specific antigen binding molecule comprises an amino acid sequence selected from: ASVNQTPRTATKETGESLTINCVLTDTSYGLYSTSWFRKNPGTTDWERMSIGGRYVESVNKRAKSFSLRI KDLTVADSATYYCKAQSGMAISTGSGHGYNWYDGAGTVLTVN (SEQ ID NO: 39); AKVDQTPRTATKETGESLTINCVLTDTSYGLYSTSWFRKNPGTTDWERMSIGGRYVESVNKRAKSFSLRI KDLTVADSATYYCKAQSGMAIDIGSGHGYNWYDGAGTVLTVN (SEQ ID NO: 40); TRVDQTPRTATKETGESLTINCVVTGAKYGLAATYWYRKNPGSSNQERISISGRYVESVNKRTMSFSLRIK DLTVADSATYYCKAYPWAMWGQWYDGAGTVLTVN (SEQ ID NO: 41); TRVDQTPRTATKETGESLTINCVVTGAKYGLFATYWYRKNPGSSNQERISISGRYVESVNKRTMSFSLRIK DLTVADSATYYCKAVFMPQHWHPAAHWYDGAGTVLTVN (SEQ ID NO: 42); TRVDQTPRTATKETGESLTINCVLTDTSYGLYSTSWFRKNPGTTDWERMSIGGRYVESVNKGAKSFSLRI KDLTVADSATYYCKAREARHPWLRQWYDGAGTVLTVN (SEQ ID NO: 43); ASVNQTPRTATKETGESLTINCVVTGANYGLAATYWYRKNPGSSNQERISISGRYVESVNKRTMSFSLRIK DLTVADSATYYCKAYPWGAGAPWLVQWYDGAGTVLTVN (SEQ ID NO: 44); TRVDQSPSSLSASVGDRVTITCVLTGANYGLASTYWYRKNPGSSNKEQISISGRYSESVNKGTKSFTLTIS SLQPEDSATYYCRAYPWGAGAPWLVQWYDGAGTKVEIK (SEQ ID NO: 45); TRVDQSPSSLSASVGDRVTITCVLTGANYGLASTYWYRKNPGSSNQERISISGRYSESVNKRTMSFTLTIS SLQPEDSATYYCRAYPWGAGAPWLVQWYDGAGTKVEIK (SEQ ID NO: 46); TRVDQSPSSLSASVGDRVTITCVLTDTSYGLYSTSWFRKNPGTTDWERMSIGGRYVESVNKGAKSFTLTI SSLQPEDFATYYCKAREARHPWLRQWYDGAGTKVEIK (SEQ ID NO: 47); TRVDQSPSSLSASVGDRVTITCVLTDTSYGLYSTYWYRKNPGSSNKEQISISGRYSESVNKGTKSFTLTIS SLQPEDSATYYCRAREARHPWLRQWYDGAGTKVEIK (SEQ ID NO: 48); TRVDQSPSSLSASVGDRVTITCVLTDTSYGLYSTYWYRKNPGTTDWERMSIGGRYSESVNKGAKSFTLTI SSLQPEDSATYYCRAREARHPWLRQWYDGAGTKVEIK (SEQ ID NO: 49), or a functional variant thereof with a sequence identity of at least 45%.

The ROR1-specific antigen binding molecule of the present invention may be humanized. The ROR1-specific antigen binding molecule of the present invention may be de-immunized. Examples of humanised sequences of the invention include, but are not limited to:

B1 G1
(SEQ ID NO: 45)
TRVDQSPSSLSASVGDRVTITCVLTGANYGLASTYWYRKNPGSSNKEQISISGRYSESVNKGTKSFTLTIS
SLQPEDSATYYCRAYPWGAGAPWLVQWYDGAGTKVEIK;
B1 G2
(SEQ ID NO: 46)
TRVDQSPSSLSASVGDRVTITCVLTGANYGLASTYWYRKNPGSSNQERISISGRYSESVNKRTMSFTLTIS
SLQPEDSATYYCRAYPWGAGAPWLVQWYDGAGTKVEIK;
P3A1 V1
(SEQ ID NO: 47)
TRVDQSPSSLSASVGDRVTITCVLTDTSYGLYSTSWFRKNPGTTDWERMSIGGRYVESVNKGAKSFTLTI
SSLQPEDFATYYCKAREARHPWLRQWYDGAGTKVEIK;
P3A1 G1
(SEQ ID NO: 48)
TRVDQSPSSLSASVGDRVTITCVLTDTSYGLYSTYWYRKNPGSSNKEQISISGRYSESVNKGTKSFTLTIS
SLQPEDSATYYCRAREARHPWLRQWYDGAGTKVEIK;
P3A1 G2
(SEQ ID NO: 49)
TRVDQSPSSLSASVGDRVTITCVLTDTSYGLYSTYWYRKNPGTTDWERMSIGGRYSESVNKGAKSFTLTI
SSLQPEDSATYYCRAREARHPWLRQWYDGAGTKVEIK;
D3 humanised ADV1
(SEQ ID NO: 50)
ASVNQSPSSLSASVGDRVTITCVLTDTSYGLYSTSWFRKNPGTTDWERMSIGGRYSESVNKGAKSFTLTI
SSLQPEDSATYYCKAQSGMAISTGSGHGYNWYDGAGTKVEIK;
D3 humanised ADV2
(SEQ ID NO: 51)
TRVDQSPSSLSASVGDRVTITCVLTDTSYGLYSTSWFRKNPGTTDWERMSIGGRYSESVNKGAKSFTLTI
SSLQPEDSATYYCKAQSGMAISTGSGHGYNWYDGAGTKVEIK;
D3 humanised ADV3
(SEQ ID NO: 52)
ASVNQSPSSASASVGDRLTITCVLTDTSYGLYSTSWFRKNPGTTDWERMSIGGRYSESVNKGAKSFTLTI
SSLQPEDSATYYCKAQSGMAISTGSGHGYNWYDGAGTKLEVK;
B1 humanised V5
(SEQ ID NO: 53)
ASVDQSPSSLSASVGDRVTITCVVTGANYGLAATYWYRKNPGSSNQERISISGRYSESVNKRTMSFTLTIS
SLQPEDSATYYCKAYPWGAGAPWLVQWYDGAGTKVEIK;
B1 humanised V7
(SEQ ID NO: 54)
ASVDQSPSSASASVGDRLTITCVVTGANYGLAATYWYRKNPGSSNQERISISGRYSESVNKRTMSFTLTIS
SLQPEDSATYYCKAYPWGAGAPWLVQWYDGAGTKLEVK;
D3 humanised EL V1
(SEQ ID NO: 55)
ASVNQSPSSLSASVGDRVTITCVLTDTSYGLYSTSWFRKNPGTTDWERMSIGGRYVESVNKRAKSFSLRI
KDLTVADSATYYCKAQSGMAISTGSGHGYNWYDGAGTKVEIK;
D3 humanised EL V2
(SEQ ID NO: 56)
ASVNQSPSSLSASVGDRVTITCVLTDTSYGLYSTSWFRKNPGTTDWERMSIGGRYVESVNKRAKSFTLTI
SSLQPEDFATYYCKAQSGMAISTGSGHGYNWYDGAGTKVEIK;
D3 humanised EL V3
(SEQ ID NO: 57)
ASVNQSPSSLSASVGDRVTITCVLTDTSYGLYSTSWFRKNPGTTDWERMSIGGRFSGSGSKRAKSFTLTI
SSLQPEDFATYYCKAQSGMAISTGSGHGYNWYDGAGTKVEIK;
D3 humanised EL V4
(SEQ ID NO: 58)
ASVNQSPSSLSASVGDRVTITCVLTDTSYGLYSTSWYQQKPGTTDWERMSIGGRYVESVNKRAKSFTLTI
SSLQPEDFATYYCKAQSGMAISTGSGHGYNWYDGAGTKVEIK;
and
D3 humanised EL V5
(SEQ ID NO: 59)
ASVNQSPSSLSASVGDRVTITCVLTDTSYGLYSTSWYQQKPGTTDWERMSIGGRFSGSGSKRAKSFTLTI
SSLQPEDFATYYCKAQSGMAISTGSGHGYNWYDGAGTKVEIK.

The ROR1-specific antigen binding molecule of the present invention may also be conjugated to a detectable label, dye, toxin, drug, pro-drug, radionuclide or biologically active molecule.

Preferably, the ROR1-specific antigen binding molecule selectively interacts with ROR1 protein with an affinity constant of approximately 0.01 to 50 nM, preferably 0.1 to 30 nM, even more preferably 0.1 to 10 nM.

Furthermore, the ROR1-specific antigen binding molecule is preferably capable of mediating killing of ROR1-expressing tumour cells or is capable of inhibiting cancer cell proliferation.

The ROR1-specific antigen binding molecule may also be capable of being endocytosed upon binding to ROR1. In other embodiments, the ROR1-specific antigen binding molecule may not be endocytosed upon binding to ROR1.

In a second aspect of the present invention, there it is provided a recombinant fusion protein comprising a specific antigen binding molecule of the first aspect. Preferably, in the recombinant fusion protein of the second aspect, the specific antigen binding molecule is fused to one or more biologically active proteins. The specific antigen binding molecule may be fused to one or more biologically active proteins via one or more linker domains. Preferred linkers include but are not limited to [G₄S]ₓ, where x is 1, 2, 3, 4, 5, or 6. Particular preferred linkers are [G₄S]₃ (SEQ ID NO: 60) and [G₄S]₅ (SEQ ID NO: 61). Other preferred linkers include the sequences PGVQPSP (SEQ ID NO: 62), PGVQPSPGGGGS (SEQ ID NO: 63) and PGVQPAPGGGGS (SEQ ID NO: 64). These linkers may be particularly useful when recombinant fusion proteins are expressed in different expression systems that differ in glycosylation patterns, such as CHO and insect, and those that do not glycosylate expressed proteins (e.g. E. coli).

It will also be appreciated that the fusion proteins of the invention can be constructed in any order, i.e., with the ROR1-specific antigen binding molecule at the N-terminus, C-terminus, or at neither terminus (e.g. in the middle of a longer amino acid sequence).

Preferred biologically active proteins include, but are not limited to an immunoglobulin, an immunoglobulin Fc region, an immunoglobulin Fab region, a single chain Fv (scFv), a diabody, a triabody, a tetrabody, a bispecific t-cell engager (BiTE), an intein, a VNAR domain, a single domain antibody (sdAb), a VH domain, or a scaffold protein (affibodies, centyrins, darpins etc.). A particularly preferred biologically active protein is an immunoglobulin Fc region. Other preferred fusion proteins include VNAR-VNAR and VNAR-VNAR-VNAR.

Any part of the fusion protein of the invention may be engineered to enable conjugation. In a preferred example, where an immunoglobulin Fc region is used, it may be engineered to include a cysteine residue as a conjugation site. Preferred introduced cysteine residues include, but are not limited to S252C and S473C (Kabat numbering), which correspond to S239C and S442C in EU numbering, respectively.

In accordance with the second aspect, recombinant fusions comprising multiple VNAR domains are provided. Accordingly, the recombinant fusions of the invention may be dimers, trimers or higher order multimers of VNARs. In such recombinant fusions, the specificity of each VNAR may be the same or different. Recombinant fusions of the invention include, but are not limited to, bi-specific or tri-specific molecules in which each VNAR domain binds to a different antigen, or to different epitopes on a single antigen (bi-paratopic binders). The term “bi-paratopic” as used herein is intended to encompass molecules that bind to multiple epitopes on a given antigen. Molecules that bind three or more eptiopes on a given antigen are also contemplated herein and where the term “bi-paratopic” is used, it should be understood that the potential for tri-paratopic or multi-paratopic molecules is also encompassed.

Also in accordance with the second aspect, recombinant fusions are provided which include a ROR1-specific antigen binding molecule of the first aspect and a humanised VNAR domain. Humanised VNAR domains may be referred to as soloMERs and include but are not limited to the VNAR BA11, which is a humanised VNAR that binds with high affinity to human serum albumin (Kovalenko et al, J. Biol. Chem., 2013 JBC).

Examples of bi-paratopic and multivalent fusion proteins include, but are not limited to:

| | | |
|---|---|---|
| B1-BA11 | P3A1-BA11-B1 | P3A1-(L₄)-BA11- |
| B1-BA11 | B1-BA11-D3 | (L₄)-D3 |
| BA11-B1 | D3-BA11-B1 | P3A1-(L₄)-D3-(L₄)- |
| 2V-BA11 | D3-BA11-P3A1 | BA11 |
| BA11-2V | P3A1-BA11-D3 | B1-(L₄)-BA11-(L₄)- |
| D3-D3-BA11 | B1G1-B1G1 | D3 |
| 2V-2V-BA11 | B1G2-B1G2 | BA11-(L₄)-B1- |
| B1-BA11 Cys | P3A1-(L₂)-B1 | (L₄)-D3 |
| B1-BA11 Cys | P3A1-(L₃)-B1 | D3-(L₄)-BA11- |
| D3-D3-BA11 Cys | B1-(L₄)-P3A1 | (L₄)-B1 |
| 2V-BA11 Cys | P3A1-(L₄)-B1 | P3A1-(L₄)-BA11- |
| D3-B1 | D3-(L₄)-P3A1 | (L₄)-P3A1 |
| B1-D3 | P3A1-(L₄)-D3 | BA11-(L₄)-P3A1- |
| D3-P3A1 | B1-(L₄)-D3 | (L₄)-P3A1 |
| P3A1-D3 | B1-(L₄)-BA11- | D3-(L₅)-P3A1 |
| D3-B1 Cys | (L₄)-P3A1 | D3-(L₅)-P3A1- |
| B1-D3 Cys | BA11-(L₄)-B1- | (L₅)-BA11 |
| D3-P3A1 Cys | (L₄)-P3A1 | D3-(L₅)-BA11- |
| P3A1-D3 Cys | P3A1-(L₄)-BA11- | (L₅)-P3A1 |
| B1-BA11-B1 | (L₄)-B1 | P3A1-(L₅)-D3 |
| D3-BA11-D3 | D3-(L₄)-P3A1- | P3A1-(L₅)-BA11- |
| P3A1-BA11-P3A1 | (L₄)-B1 | (L₅)-D3 |
| E9-BA11-E9 | D3-(L₄)-BA11- | P3A1-(L₅)-D3- |
| B1-BA11-P3A1 | (L₄)-P3A1 | (L₅)-BA11 |
| | | 2V-(L₄)-2V |
| | | 2V-(L₄)-BA11- |
| | | (L₄)-2V |

Wherein:

B1 is
(SEQ ID NO: 44)
ASVNQTPRTATKETGESLTINCVVTGANYGLAATYWYRKNPGSSNQERI
SISGRYVESVNKRTMSFSLRIKDLTVADSATYYCKAYPWGAGAPWLVQW
YDGAGTVLTVN
2V is
(SEQ ID NO: 65)
TRVDQTPRTATKETGESLTINCVLTDTSYGLYSTSWFRKNPGTTDWERM
SIGGRYVESVNKGAKSFSLRIKDLTVADSATYYCKAQSLAISTRSYWYD
GAGTVLTVN
P3A1 is
(SEQ ID NO: 43)
TRVDQTPRTATKETGESLTINCVLTDTSYGLYSTSWFRKNPGTTDWERM
SIGGRYVESVNKGAKSFSLRIKDLTVADSATYYCKAREARHPWLRQWYD
GAGTVLTVN
D3 is
(SEQ ID NO: 39)
ASVNQTPRTATKETGESLTINCVLTDTSYGLYSTSWFRKNPGTTDWERM
SIGGRYVESVNKRAKSFSLRIKDLTVADSATYYCKAQSGMAISTGSGHG
YNWYDGAGTVLTVN
BA11; is
(SEQ ID NO: 66)
TRVDQSPSSLSASVGDRVTITCVLTDTSYPLYSTYWYRKNPGSSNKEQI
SISGRYSESVNKGTKSFTLTISSLQPEDSATYYCRAMSTNIWTGDGAGT
KVEIK
E9 is
(SEQ ID NO: 40)
AKVDQTPRTATKETGESLTINCVLTDTSYGLYSTSWFRKNPGTTDWERM
SIGGRYVESVNKRAKSFSLRIKDLTVADSATYYCKAQSGMAIDIGSGHG
YNWYDGAGTVLTVN
B1G2 is
(SEQ ID NO: 46)
TRVDQSPSSLSASVGDRVTITCVLTGANYGLASTYWYRKNPGSSNQERI
SISGRYSESVNKRTMSFTLTISSLQPEDSATYYCRAYPWGAGAPWLVQW
YDGAGTKVEIK
B1G1;
(SEQ ID NO: 45)
TRVDQSPSSLSASVGDRVTITCVLTGANYGLASTYWYRKNPGSSNKEQI
SISGRYSESVNKGTKSFTLTISSLQPEDSATYYCRAYPWGAGAPWLVQW
YDGAGTKVEIK

and
Where no linker is defined (-) corresponds to a linker of -(G₄S)₅—
-(L₂)- corresponds to a linker of -(G₄S)₃—
-(L₃)- corresponds to a linker of -(G₄S)₇—
-(L₄)- corresponds to the linker Wobbe-G₄S, which in turn is PGVQPSPGGGGS (SEQ ID NO: 63)
-(L₅)- corresponds to the linker Wobbe-G₄S-GM, which in turn is PGVQPAPGGGGS (SEQ ID NO: 64)
Cys—corresponds to a Cys containing C-terminal tag—for example QACGAHHHHHHGAEFEQKLISEEDL (SEQ ID NO: 67)

In certain embodiments, the specific binding molecules or recombinant fusions of the invention may be expressed with N- or C-terminal tags to assist with purification. Examples include but are not limited to His₆ and/or Myc. In addition, the N- or C-terminal tag may be further engineered to include additional cysteine residues to serve as conjugation points. It will therefore be appreciated that reference to specific binding molecules or recombinant fusions in all aspects of the invention is also intended to encompass such molecules with a variety of N- or C-terminal tags, which tags may also include additional cysteines for conjugation.

Additional recombinant fusions are listed below. It will be appreciated that not every combination of linker and VNAR or fusion partner is listed below. However, all such combinations are expressly encompassed by the present invention.

Monovalent-B
A11 fusions
BA11-B1
B1-BA11
P3A1-BA11
BA11-P3A1
D3-BA11
BA11-D3
E9-BA11
BA11-E9
Divalent-
BA11 fusions
P3A1-P3A1-BA11
BA11-P3A1-P3A1
P3A1-BA11-P3A1
D3-D3-BA11
D3-BA11-D3
BA11-D3-D3
B1-B1-BA11
B1-BA11-B1
BA11-B1-B1
E9-E9-BA11
E9-BA11-E9
BA11-E9-E9
Biparatopic
Dimers
B1-P3A1
P3A1-B1
B1-D3
D3-B1
D3-P3A1
P3A1-D3
E9-B1
B1-E9
E9-P3A1
P3A1-E9
E9-D3
D3-E9
Dimeric biparatopic
BA11 fusions
B1-D3-BA11
D3-B1-BA11
B1-BA11-D3
D3-BA11-B1
BA11-B1-D3
BA11-D3-B1
B1-P3A1-BA11
P3A1-B1-BA11
B1-BA11-P3A1
P3A1-BA11-B1
BA11-B1-P3A1
BA11-P3A1-B1
D3-P3A1-BA11
P3A1-D3-BA11
D3-BA11-P3A1
P3A1-BA11-D3
BA11-D3-P3A1
BA11-P3A1-D3
D3-E9-BA11
E9-D3-BA11
E9-BA11-D3
D3-BA11-E9
BA11-D3-E9
BA11-E9-D3
E9-P3A1-BA11
P3A1-E9-BA11
E9-BA11-P3A1
P3A1-BA11-E9
BA11-E9-P3A1
BA11-P3A1-E9
B1-E9-BA11
E9-B1-BA11
B1-BA11-E9
E9-BA11-B1
BA11-B1-E9
BA11-E9-B1
Trimeric-
Biparatopics
B1-B1-D3
B1-D3-B1
D3-B1-B1
B1-B1-P3A1
B1-P3A1-B1
P3A1-B1-B1
B1-B1-E9
B1-E9-B1
E9-B1-B1
D3-D3-P3A1
D3-P3A1-D3
P3A1-D3-D3
D3-D3-E9
D3-E9-D3
E9-D3-D3
D3-D3-B1
D3-B1-D3
B1-D3-D3
P3A1-P3A1-B1
P3A1-B1-P3A1
B1-P3A1-P3A1
P3A1-P3A1-D3
P3A1-D3-P3A1
D3-P3A1-P3A1
P3A1-P3A1-E9
P3A1-E9-P3A1
E9-P3A1-P3A1
E9-E9-B1
E9-B1-E9
B1-E9-E9
E9-E9-P3A1
E9-P3A1-E9
P3A1-E9-E9
E9-E9-D3
E9-D3-E9
D3-E9-E9

Where the linkers between the VNAR domains are preferentially, but not limited to (G₄S)₅, (G₄S)₃, (G₄S)₇, PGVQPSPGGGGS (SEQ ID NO: 63) (Wobbe-G₄S), PGVQPAPGGGGS (SEQ ID NO: 64) (Wobbe-G₄S GM) and wherein different combinations of different linkers can be combined within the same construct.

Whereby, additional C-terminal (or N-terminal) tag sequences may or may not be present. C-terminal tags include, but are not limited to, tags that contain poly-Histidine sequences to facilitate purification (such as His₆), contain c-Myc sequences (such as EQKLISEEDL (SEQ ID NO: 68)) to enable detection and/or contain Cysteine residues to enable labelling and bioconjugation using thiol reactive payloads and probes and combinations thereof. Preferential C-terminal tags include but are not limited to:

(SEQ ID NO: 69)
QASGAHHHHHHGAEFEQKLISEEDL
(SEQ ID NO: 67)
QACGAHHHHHHGAEFEQKLISEEDL
(SEQ ID NO: 70)
QACKAHHHHHHGAEFEQKLISEEDL
(SEQ ID NO: 71)
AAAHHHHHHGAEFEQKLISEEDL
(SEQ ID NO: 72)
ACAHHHHHHGAEFEQKLISEEDL
(SEQ ID NO: 73)
QASGAHHHHHH
(SEQ ID NO: 74)
QACGAHHHHHH
(SEQ ID NO: 75)
QACKAHHHHHH
(SEQ ID NO: 76)
AAAHHHHHH
(SEQ ID NO: 77)
ACAHHHHHH
(SEQ ID NO: 78)
QASGA
(SEQ ID NO: 79)
QACGA
(SEQ ID NO: 80)
QACKA
(SEQ ID NO: 81)
ACA
(SEQ ID NO: 82)
SAPSA

Wherein:

•B1 is
(SEQ ID NO: 44)
ASVNQTPRTATKETGESLTINCVVTGANYGLAATYWYRKNPGSSNQERI
SISGRYVESVNKRTMSFSLRIKDLTVADSATYYCKAYPWGAGAPWLVQW
YDGAGTVLTVN
•2V is
(SEQ ID NO: 65)
TRVDQTPRTATKETGESLTINCVLTDTSYGLYSTSWFRKNPGTTDWERM
SIGGRYVESVNKGAKSFSLRIKDLTVADSATYYCKAQSLAISTRSYWYD
GAGTVLTVN
•P3A1 is
(SEQ ID NO: 43)
TRVDQTPRTATKETGESLTINCVLTDTSYGLYSTSWFRKNPGTTDWERM
SIGGRYVESVNKGAKSFSLRIKDLTVADSATYYCKAREARHPWLRQWYD
GAGTVLTVN
•D3 is
(SEQ ID NO: 39)
ASVNQTPRTATKETGESLTINCVLTDTSYGLYSTSWFRKNPGTTDWERM
SIGGRYVESVNKRAKSFSLRIKDLTVADSATYYCKAQSGMAISTGSGHG
YNWYDGAGTVLTVN
•BA11; is
(SEQ ID NO: 66)
TRVDQSPSSLSASVGDRVTITCVLTDTSYPLYSTYWYRKNPGSSNKEQI
SISGRYSESVNKGTKSFTLTISSLQPEDSATYYCRAMSTNIWTGDGAGT
KVEIK
•E9 is
(SEQ ID NO: 40)
AKVDQTPRTATKETGESLTINCVLTDTSYGLYSTSWFRKNPGTTDWERM
SIGGRYVESVNKRAKSFSLRIKDLTVADSATYYCKAQSGMAIDIGSGHG
YNWYDGAGTVLTVN

As stated above, all combinations of VNAR and linker are expressly encompassed herein.

Humanised derivatives of the VNARs are also encompassed herein.

Also in accordance with the second aspect, recombinant fusions are provided which include a ROR1-specific antigen binding molecule of the first aspect and a recombinant toxin. Examples of recombinant toxins include but are not limited to Pseudomonas exotoxin PE38 and diphtheria toxin.

Also in accordance with the second aspect, recombinant fusions are provided which include a ROR1-specific antigen binding molecule of the first aspect and a recombinant CD3 binding protein. Examples of recombinant ROR1 and CD3 binding agents include but are not limited to:

B1 CD3
(SEQ ID NO: 83)
ASVNQTPRTATKETGESLTINCVVTGANYGLAATYWYRKNPGSSNQERISISGRYVESVNKRTMSFSLRIK
DLTVADSATYYCKAYPWGAGAPWLVQWYDGAGTVLTVNGGGGSDIKLQQSGAELARPGASVKMSCKTS
GYTFTRYTMHWVKQRPGQGLEWIGYINPSRGYTNYNQKFKDKATLTTDKSSSTAYMQLSSLTSEDSAVY
YCARYYDDHYCLDYWGQGTTLTVSSGGGGSGGGGSGGGGSDIQLTQSPAIMSASPGEKVTMTCRASS
SVSYMNWYQQKSGTSPKRWIYDTSKVASGVPYRFSGSGSGTSYSLTISSMEAEDAATYYCQQWSSNPL
TFGAGTKLELKSHHHHHH
B1 CD3[G₄S]₃
(SEQ ID NO: 84)
ASVNQTPRTATKETGESLTINCVVTGANYGLAATYWYRKNPGSSNQERISISGRYVESVNKRTMSFSLRIK
DLTVADSATYYCKAYPWGAGAPWLVQWYDGAGTVLTVNGGGGSGGGGSGGGGSDIKLQQSGAELARP
GASVKMSCKTSGYTFTRYTMHWVKQRPGQGLEWIGYINPSRGYTNYNQKFKDKATLTTDKSSSTAYMQL
SSLTSEDSAVYYCARYYDDHYCLDYWGQGTTLTVSSGGGGSGGGGSGGGGSDIQLTQSPAIMSASPGE
KVTMTCRASSSVSYMNWYQQKSGTSPKRWIYDTSKVASGVPYRFSGSGSGTSYSLTISSMEAEDAATYY
CQQWSSNPLTFGAGTKLELKSHHHHHH
P3A1 CD3
(SEQ ID NO: 85)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNGGGGSDIKLQQSGAELARPGASVKMSCKTSG
YTFTRYTMHWVKQRPGQGLEWIGYINPSRGYTNYNQKFKDKATLTTDKSSSTAYMQLSSLTSEDSAVYY
CARYYDDHYCLDYWGQGTTLTVSSGGGGSGGGGSGGGGSDIQLTQSPAIMSASPGEKVTMTCRASSS
VSYMNWYQQKSGTSPKRWIYDTSKVASGVPYRFSGSGSGTSYSLTISSMEAEDAATYYCQQWSSNPLT
FGAGTKLELKSHHHHHH
P3A1 CD3[G₄S]₃
(SEQ ID NO: 86)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNGGGGSGGGGSGGGGSDIKLQQSGAELARPG
ASVKMSCKTSGYTFTRYTMHWVKQRPGQGLEWIGYINPSRGYTNYNQKFKDKATLTTDKSSSTAYMQLS
SLTSEDSAVYYCARYYDDHYCLDYWGQGTTLTVSSGGGGSGGGGSGGGGSDIQLTQSPAIMSASPGEK
VTMTCRASSSVSYMNWYQQKSGTSPKRWIYDTSKVASGVPYRFSGSGSGTSYSLTISSMEAEDAATYYC
QQWSSNPLTFGAGTKLELKSHHHHHH
P3A1 P3A1 CD3
(SEQ ID NO: 87)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNGGGGSGGGGSGGGGSGGGGSGGGGSTRV
DQTPRTATKETGESLTINCVLTDTSYGLYSTSWFRKNPGTTDWERMSIGGRYVESVNKGAKSFSLRIKDL
TVADSATYYCKAREARHPWLRQWYDGAGTVLTVNGGGGSDIKLQQSGAELARPGASVKMSCKTSGYTF
TRYTMHWVKQRPGQGLEWIGYINPSRGYTNYNQKFKDKATLTTDKSSSTAYMQLSSLTSEDSAVYYCAR
YYDDHYCLDYWGQGTTLTVSSGGGGSGGGGSGGGGSDIQLTQSPAIMSASPGEKVTMTCRASSSVSY
MNWYQQKSGTSPKRWIYDTSKVASGVPYRFSGSGSGTSYSLTISSMEAEDAATYYCQQWSSNPLTFGA
GTKLELKSHHHHHH
P3A1 P3A1 CD3[G₄S]₃
(SEQ ID NO: 88)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNGGGGSGGGGSGGGGSGGGGSGGGGSTRV
DQTPRTATKETGESLTINCVLTDTSYGLYSTSWFRKNPGTTDWERMSIGGRYVESVNKGAKSFSLRIKDL
TVADSATYYCKAREARHPWLRQWYDGAGTVLTVNGGGGSGGGGSGGGGSDIKLQQSGAELARPGASV
KMSCKTSGYTFTRYTMHWVKQRPGQGLEWIGYINPSRGYTNYNQKFKDKATLTTDKSSSTAYMQLSSLT
SEDSAVYYCARYYDDHYCLDYWGQGTTLTVSSGGGGSGGGGSGGGGSDIQLTQSPAIMSASPGEKVT
MTCRASSSVSYMNWYQQKSGTSPKRWIYDTSKVASGVPYRFSGSGSGTSYSLTISSMEAEDAATYYCQ
QWSSNPLTFGAGTKLELKSHHHHHH
P3A1-[PGVQPSPGGGGS]-B1-[G₄S]-CD3
(SEQ ID NO: 89)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNPGVQPSPGGGGSASVNQTPRTATKETGESLT
INCVVTGANYGLAATYWYRKNPGSSNQERISISGRYVESVNKRTMSFSLRIKDLTVADSATYYCKAYPWG
AGAPWLVQWYDGAGTVLTVNGGGGSDIKLQQSGAELARPGASVKMSCKTSGYTFTRYTMHWVKQRPG
QGLEWIGYINPSRGYTNYNQKFKDKATLTTDKSSSTAYMQLSSLTSEDSAVYYCARYYDDHYCLDYWGQ
GTTLTVSSGGGGSGGGGSGGGGSDIQLTQSPAIMSASPGEKVTMTCRASSSVSYMNWYQQKSGTSPK
RWIYDTSKVASGVPYRFSGSGSGTSYSLTISSMEAEDAATYYCQQWSSNPLTFGAGTKLELKSHHHHHH
P3A1-[PGVQPSPGGGGS]-B1-[G₄S]₃-CD3
(SEQ ID NO: 90)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNPGVQPSPGGGGSASVNQTPRTATKETGESLT
INCVVTGANYGLAATYWYRKNPGSSNQERISISGRYVESVNKRTMSFSLRIKDLTVADSATYYCKAYPWG
AGAPWLVQWYDGAGTVLTVNGGGGSGGGGSGGGGSDIKLQQSGAELARPGASVKMSCKTSGYTFTRY
TMHWVKQRPGQGLEWIGYINPSRGYTNYNQKFKDKATLTTDKSSSTAYMQLSSLTSEDSAVYYCARYYD
DHYCLDYWGQGTTLTVSSGGGGGGGGSGGGGSDIQLTQSPAIMSASPGEKVTMTCRASSSVSYMNW
YQQKSGTSPKRWIYDTSKVASGVPYRFSGSGSGTSYSLTISSMEAEDAATYYCQQWSSNPLTFGAGTKL
ELKSHHHHHH
P3A1-[PGVQPAPGGGGS]-D3-[G₄S]-CD3
(SEQ ID NO: 91)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNPGVQPAPGGGGSASVNQTPRTATKETGESLT
INCVLTDTSYGLYSTSWFRKNPGTTDWERMSIGGRYVESVNKRAKSFSLRIKDLTVADSATYYCKAQSGM
AISTGSGHGYNWYDGAGTVLTVNGGGGSDIKLQQSGAELARPGASVKMSCKTSGYTFTRYTMHWVKQR
PGQGLEWIGYINPSRGYTNYNQKFKDKATLTTDKSSSTAYMQLSSLTSEDSAVYYCARYYDDHYCLDYW
GQGTTLTVSSGGGGSGGGGSGGGGSDIQLTQSPAIMSASPGEKVTMTCRASSSVSYMNWYQQKSGTS
PKRWIYDTSKVASGVPYRFSGSGSGTSYSLTISSMEAEDAATYYCQQWSSNPLTFGAGTKLELKSHHHH
HH
P3A1-[PGVQPAPGGGGS]-D3-[G₄S]₃-CD3
(SEQ ID NO: 92)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNPGVQPAPGGGGSASVNQTPRTATKETGESLT
INCVLTDTSYGLYSTSWFRKNPGTTDWERMSIGGRYVESVNKRAKSFSLRIKDLTVADSATYYCKAQSGM
AISTGSGHGYNWYDGAGTVLTVNGGGGSGGGGSGGGGSDIKLQQSGAELARPGASVKMSCKTSGYTF
TRYTMHWVKQRPGQGLEWIGYINPSRGYTNYNQKFKDKATLTTDKSSSTAYMQLSSLTSEDSAVYYCAR
YYDDHYCLDYWGQGTTLTVSSGGGGSGGGGSGGGGSDIQLTQSPAIMSASPGEKVTMTCRASSSVSY
MNWYQQKSGTSPKRWIYDTSKVASGVPYRFSGSGSGTSYSLTISSMEAEDAATYYCQQWSSNPLTFGA
GTKLELKSHHHHHH
P3A1-[G₄S]₅-D3-[G₄S]-CD3
(SEQ ID NO: 93)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNGGGGSGGGGGGGGSGGGGSGGGGSASV
NQTPRTATKETGESLTINCVLTDTSYGLYSTSWFRKNPGTTDWERMSIGGRYVESVNKRAKSFSLRIKDL
TVADSATYYCKAQSGMAISTGSGHGYNWYDGAGTVLTVNGGGGSDIKLQQSGAELARPGASVKMSCKT
SGYTFTRYTMHWVKQRPGQGLEWIGYINPSRGYTNYNQKFKDKATLTTDKSSSTAYMQLSSLTSEDSAV
YYCARYYDDHYCLDYWGQGTTLTVSSGGGGSGGGGSGGGGSDIQLTQSPAIMSASPGEKVTMTCRAS
SSVSYMNWYQQKSGTSPKRWIYDTSKVASGVPYRFSGSGSGTSYSLTISSMEAEDAATYYCQQWSSNP
LTFGAGTKLELKSHHHHHH
P3A1-[G4S]₅-D3-[G₄S]₃-CD3
(SEQ ID NO: 94)
TRVDQTPRTATKETGESLTINCVLTDTSYGLYSTSWFRKNPGTTDWERMSIGGRYVESVNKGAKSFSLRI
KDLTVADSATYYCKAREARHPWLRQWYDGAGTVLTVNGGGGSGGGGSGGGGSGGGGSGGGGSASV
NQTPRTATKETGESLTINCVLTDTSYGLYSTSWFRKNPGTTDWERMSIGGRYVESVNKRAKSFSLRIKDL
TVADSATYYCKAQSGMAISTGSGHGYNWYDGAGTVLTVNGGGGSGGGGSGGGGSDIKLQQSGAELAR
PGASVKMSCKTSGYTFTRYTMHWVKQRPGQGLEWIGYINPSRGYTNYNQKFKDKATLTTDKSSSTAYM
QLSSLTSEDSAVYYCARYYDDHYCLDYWGQGTTLTVSSGGGGSGGGGSGGGGSDIQLTQSPAIMSASP
GEKVTMTCRASSSVSYMNWYQQKSGTSPKRWIYDTSKVASGVPYRFSGSGSGTSYSLTISSMEAEDAAT
YYCQQWSSNPLTFGAGTKLELKSHHHHHH

In a third aspect of the present invention, there is provided a ROR1-specific chimeric antigen receptor (CAR), comprising at least one ROR1-specific antigen binding molecule as defined by the first aspect of the invention, fused or conjugated to at least one transmembrane region and at least one intracellular domain.

The present invention also provides a cell comprising a chimeric antigen receptor according to the third aspect, which cell is preferably an engineered T-cell.

In a fourth aspect of the invention, there is provided a nucleic acid sequence comprising a polynucleotide sequence that encodes a specific antigen binding molecule, recombinant fusion protein or chimeric antigen receptor according to the first, second or third aspects of the invention.

There is also provided a vector comprising a nucleic acid sequence in accordance with the fourth aspect and a host cell comprising such a nucleic acid.

A method for preparing a specific antigen binding molecule, recombinant fusion protein or chimeric antigen receptor, of the first, second or third aspect is provided, the method comprising cultivating or maintaining a host cell comprising the polynucleotide or vector described above under conditions such that said host cell produces the specific antigen binding molecule, recombinant fusion protein or chimeric antigen receptor, optionally further comprising isolating the specific antigen binding molecule, recombinant fusion protein or chimeric antigen receptor.

In a fifth aspect of the invention, there is provided a pharmaceutical composition comprising the specific antigen binding molecule, fusion protein or chimeric antigen receptor of the first, second or third aspects. The pharmaceutical composition may contain a variety of pharmaceutically acceptable carriers. Pharmaceutical compositions of the invention may be for administration by any suitable method known in the art, including but not limited to intravenous, intramuscular, oral, intraperitoneal, or topical administration. In preferred embodiments, the pharmaceutical composition may be prepared in the form of a liquid, gel, powder, tablet, capsule, or foam.

The specific antigen binding molecule, recombinant fusion protein or chimeric antigen receptor of the first, second or third aspects may be for use in therapy. More specifically, the specific antigen binding molecule, recombinant fusion protein or chimeric antigen receptor of the first, second or third aspects may be for use in the treatment of cancer. Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer.

Also provided herein is the use of a specific antigen binding molecule, recombinant fusion protein or chimeric antigen receptor of the first, second or third aspects in the manufacture of a medicament for the treatment of a disease in a patient in need thereof.

Furthermore, in accordance with the present invention there is provided a method of treatment of a disease in a patient in need of treatment comprising administration to said patient of a therapeutically effective dosage of a specific antigen binding molecule, recombinant fusion protein or chimeric antigen receptor of the first, second or third aspects or a pharmaceutical composition of the fifth aspect.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer.

Also provided herein is a method of assaying for the presence of a target analyte in a sample, comprising the addition of a detectably labelled specific antigen binding molecule of the first aspect, or a recombinant fusion protein of the second aspect, to the sample and detecting the binding of the molecule to the target analyte.

In addition, there is provided herein a method of imaging a site of disease in a subject, comprising administration of a detectably labelled specific antigen binding molecule of the first aspect or a detectably labelled recombinant fusion protein of the second aspect to a subject.

There is also provided herein a method of diagnosis of a disease or medical condition in a subject comprising administration of a specific antigen binding molecule of the first aspect or a recombinant fusion protein of the second aspect.

Also contemplated herein is an antibody, antibody fragment or antigen-binding molecule that competes for binding to ROR1 with the ROR1-specific antigen binding molecule of the first aspect. The term “compete” when used in the context of antigen binding proteins (e.g., neutralizing antigen binding proteins or neutralizing antibodies) means competition between antigen binding proteins as determined by an assay in which the antigen binding protein (e.g., antibody or functional fragment thereof) under test prevents or inhibits specific binding of a the antigen binding molecule defined herein (e.g., specific antigen binding molecule of the first aspect) to a common antigen (e.g., ROR1 in the case of the specific antigen binding molecule of the first aspect).

Also described herein is a kit for diagnosing a subject suffering from cancer, or a pre-disposition thereto, or for providing a prognosis of the subject's condition, the kit comprising detection means for detecting the concentration of antigen present in a sample from a test subject, wherein the detection means comprises a ROR1-specific antigen binding molecule of the first aspect, a recombinant fusion protein of the second aspect, a chimeric antigen receptor of the third aspect or a nucleic acid sequence of the fourth aspect, each being optionally derivatized, wherein presence of antigen in the sample suggests that the subject suffers from cancer. Preferably the antigen comprises ROR1 protein, more preferably an extracellular domain thereof. More preferably, the kit is used to identify the presence or absence of ROR1-positive cells in the sample, or determine the concentration thereof in the sample. The kit may also comprise a positive control and/or a negative control against which the assay is compared and/or a label which may be detected.

The present invention also provides a method for diagnosing a subject suffering from cancer, or a pre-disposition thereto, or for providing a prognosis of the subject's condition, the method comprising detecting the concentration of antigen present in a sample obtained from a subject, wherein the detection is achieved using a ROR1-specific antigen binding molecule of the first aspect, a recombinant fusion protein of the second aspect, a chimeric antigen receptor of the third aspect or a nucleic acid sequence of the fourth aspect, each being optionally derivatized, and wherein presence of antigen in the sample suggests that the subject suffers from cancer.

Also contemplated herein is a method of killing or inhibiting the growth of a cell expressing ROR1 in vitro or in a patient, which method comprises administering to the cell a pharmaceutically effective amount or dose of (i) ROR1-specific antigen binding molecule of the first aspect, a recombinant fusion protein of the second aspect, a nucleic acid sequence of the third aspect, or the CAR or cell according the fourth aspect, or (ii) of a pharmaceutical composition of the fifth aspect. Preferably, the cell expressing ROR1 is a cancer cell. More preferably, the ROR1 is human ROR1.

In a sixth aspect of the present invention, there is provided a specific antigen binding molecule comprising an amino acid sequence represented by the formula (II):

X-FW1-CDR1-FW2-HV2-FW3a-HV4-FW3b-CDR3-FW4-Y (II)
wherein
   FW1 is a framework region
   CDR1 is a CDR sequence
   FW2 is a framework region
   HV2 is a hypervariable sequence
   FW3a is a framework region
   HV4 is a hypervariable sequence
   FW3b is a framework region
   CDR3 is a CDR sequence
   FW4 is a framework region
   X and Y are optional amino acid sequences
wherein the specific antigen binding molecule is conjugated to a second moiety.

In certain preferred embodiments, the specific antigen binding molecule according to this aspect of the invention may additionally be conjugated to a third, fourth or fifth moiety. Conjugation of further moieties is also contemplated. In some cases, a third, fourth or fifth moiety may be conjugated to the second moiety. Accordingly, it will be understood that any of the moieties according to this aspect of the invention may have additional moieties conjugated thereto. Description of preferred features of the second moiety as set out below apply to the third, fourth, fifth or higher order moiety mutatis mutandis.

Preferably X or Y are individually either absent or selected from the group comprising an immunoglobulin, an immunoglobulin Fc region, an immunoglobulin Fab region, a single chain Fv (scFv), a diabody, a triabody, a tetrabody, a bispecific t-cell engager (BiTE), an intein, a VNAR domain, a single domain antibody (sdAb), a VH domain, a scaffold protein (affibodies, centyrins, darpins etc.), or a toxin including but not limited to Pseudomonas exotoxin PE38, diphtheria toxin.

Preferably, the conjugation is via a cysteine residue in the amino acid sequence of the specific antigen binding molecule. The cysteine residue may be anywhere in the sequence, including in optional sequences X or Y (if present).

The conjugation may be via a thiol, aminoxy or hydrazinyl moiety incorporated at the N-terminus or C-terminus of the amino acid sequence of the specific antigen binding molecule.

Preferably, the second moiety is selected from the group comprising detectable label, dye, toxin, drug, pro-drug, radionuclide or biologically active molecule.

More preferably, the second moiety is at least one toxin selected from the group comprising:
maytansinoids,
auristatins,
anthracyclins, preferably PNU-derived anthracyclins
calicheamicins,
amanitin derivatives, preferably α-amanitin derivatives
tubulysins
duocarmycins
radioisotopes for example alpha-emitting radionuclide, such as 227 Th or 225 Ac
liposomes comprising a toxic payload,
protein toxins
taxanes,
pyrrolbenzodiazepines
indolinobenzodiazepine pseudodimers and/or
spliceosome inhibitors
CDK11 inhibitors
Pyridinobenzodiazepines

In other preferred embodiments in accordance with this aspect, the second moiety may be from the group comprising an immunoglobulin, an immunoglobulin Fc region, an immunoglobulin Fab region, a single chain Fv (scFv), a diabody, a triabody, a tetrabody, a bispecific t-cell engager (BiTE), an intein, a VNAR domain, a single domain antibody (sdAb), a VH domain, a scaffold protein (affibodies, centyrins, darpins etc.), or a toxin including but not limited to Pseudomonas exotoxin PE38, diphtheria toxin.

In particularly preferred embodiments, the second moiety is a VNAR domain, which may be the same or different to the specific antigen binding molecule according to this aspect. Accordingly, dimers, trimers or higher order multimers of VNAR domains linked by chemical conjugation are explicitly contemplated herein. In such embodiments, each individual VNAR domain may have the same antigen specificity as the other VNAR domains, or they may be different.

In accordance with this aspect, the specific antigen binding molecule may comprise, for example, bi-paratopic specific antigen binding molecules as described in relation to the first to fifth aspects fused to further biologically active molecules (including but not limited to molecules for half-life extension, for example BA11) and then further conjugated to a second moiety, including but not limited to cytotoxic payloads

In accordance with this aspect, the specific antigen binding molecule may be a receptor tyrosine kinase-like orphan receptor 1 (ROR1) specific antigen binding molecule. This may be a ROR1-specific antigen binding molecule of the first aspect of the invention. Accordingly, any of the preferred features described above in relation to the first, second and third aspects apply mutatis mutandis to the sixth aspect.

The specific antigen binding molecule of the sixth aspect may be for use in therapy. More specifically, the specific antigen binding molecule of the sixth aspect may be for use in the treatment of cancer. Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer.

Also provided herein is the use of a specific antigen binding molecule of the sixth aspect in the manufacture of a medicament for the treatment of a disease in a patient in need thereof.

Pharmaceutical compositions comprising the specific antigen binding molecule of the sixth aspect are also provided. The pharmaceutical composition may contain a variety of pharmaceutically acceptable carriers

Furthermore, in accordance with the present invention there is provided a method of treatment of a disease in a patient in need of treatment comprising administration to said patient of a therapeutically effective dosage of a specific antigen binding molecule of the sixth aspect or a pharmaceutical composition comprising a specific antigen binding molecule of the sixth aspect.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer.

Also provided herein is a method of assaying for the presence of a target analyte in a sample, comprising the addition of a detectably labelled specific antigen binding molecule of the sixth aspect to the sample and detecting the binding of the molecule to the target analyte.

In addition, there is provided herein a method of imaging a site of disease in a subject, comprising administration of a detectably labelled specific antigen binding molecule of the sixth aspect to a subject.

There is also provided herein a method of diagnosis of a disease or medical condition in a subject comprising administration of a specific antigen binding molecule of the sixth aspect.

Furthermore, any of the features described in respect of any of the above-mentioned aspects of the invention may be combined mutatis mutandis with the other aspects of the invention.

### DESCRIPTION OF FIGURES

FIG. 1: anti-ROR1 phage monoclonals displaying VNAR domains: binding to human or mouse recombinant ROR1-Fc in ELISA. B1, P3A1 and E7—specific ROR1 binders, H2—non-specific phage.

FIG. 2: ROR1 binding sequences obtained from screening the synthetic VNAR library using human ROR1 (B1 and E7) and mouse ROR1 (P3A1 and CPF7). Sequences shown without and with the C-terminal His6Myc tag (His6 Myc sequence in italics).

FIG. 3: Generation of the immunised VNAR library using human ROR1: analysis of three spiny dogfish pre- and post-immunisation plasma binding to murine or human ROR1.

FIG. 4: anti-ROR1 phage monoclonals from immunised VNAR library: binding to human or mouse recombinant ROR1-Fc in ELISA. E9 and D3—specific ROR1 binders, H1—non-specific VNAR binder displayed on phage.

FIG. 5: ROR1 binding sequences E9 and D3 obtained from screening the immunised VNAR library using mouse ROR1. Sequences shown without and with the C-terminal His6Myc tag (His6 Myc sequence in italics).

FIG. 6: Far UV CD spectra of VNAR no tag, VNAR 6×His and VNAR-His6-Myc in 50 mM NaCl 20 mM NaP buffer pH 6.0 at room temperature.

FIG. 7: VNAR reformatting A: monomeric VNAR, B: homodimers, C: conjugated homodimers via C-terminal intermolecular disulphide bond, D: heterodimers, E: VNAR IgG Fc fusions, F: IgG Fc-VNAR fusions, G: VNAR-(IgG Fc)-VNAR fusions.

FIG. 8: Binding of B1 C-terminally linked homodimer to hROR1. B1, B1 C-terminal thiol (B1 SH) and B1 C-terminal disulphide dimer (B1 S—S B1) binding to human ROR1 by ELISA.

FIG. 9: Cell surface binding of VNAR (His6Myc tag) molecules to A549 (ROR1 hi) lung cancer cells by flow cytometry. B1 and E7 monomers and P3A1-P3A1 dimer bind strongly to A549 cells at all concentrations tested. CPF7 and P3A1 monomers bind at 50 μg/ml to A549 cells. VNAR binding was detected using PE-anti Myc tag Ab (CST) and analysed using a BD Biosciences FACSCalibur flow cytometer.

FIG. 10: Linker mouse IgG and linker human IgG sequences used in VNAR IgG Fc fusion proteins. Engineered hIgG1 Fc fusion proteins incorporate an engineered cysteine substitution in the hIgG1 Fc sequence, for example at position S252C or S473C (Kabat numbering) to enable site specific labelling.

FIG. 11: Intein cleavage reagents and the corresponding VNAR C-terminal derivatives.

FIG. 12: VNAR binding to human, mouse and rat ROR1 and human ROR2 by ELISA. All VNARs were found to be species cross-reactive to ROR1. None of the VNAR clones cross-reacted with human ROR2.

FIG. 13: VNAR cell surface binding to A549 (ROR1 hi) vs A427 (ROR1 low) lung cancer cell lines by flow cytometry.

FIG. 14: Cell surface binding of VNARs to MDA-MB-231 breast cancer cells for 2 hrs at 4° C. or 37° C. Loss of cell surface signal at 37° C. is suggestive of ROR1 internalisation. VNAR binding was detected using PE-anti Myc tag Ab (CST) and analysed using a BD Biosciences FACS Calibur (B1) or a ThermoFisher Attune NxT flow cytometer.

FIG. 15: Bar chart depicting VNAR-hFc molecule cell surface binding to A549 (ROR1 hi) vs A427 (ROR1 low) lung cancer cell lines. VNAR hFc binding was detected using a PE-anti-human antibody (Jackson ImmunoResearch Labs/Stratech) and a ThermoFisher Attune NxT flow cytometer.

FIG. 16: Internalisation of VNAR-Fc fusions. Cell surface binding of VNAR-Fc to MDA-MB-231 breast cancer cells for 2 hrs at 4° C. or 37° C. Loss of cell surface signal at 37° C. is suggestive of ROR1 internalisation.

FIG. 17: VNARs bind to human ROR1 independent of glycosylation. A, SDS PAGE analysis of hROR1 (lane 2) and deglycosylated hROR1 (lane 3). Mwt markers (lane 1). B, ROR1 binding VNARs B1, P3A1-P3A1 and D3-D3 bind equally well to deglycosylated hROR1 by ELISA. C, B1 mFc binds equally well to glycosylated and deglycosylated hROR1 by ELISA. Binding to unfolded hROR1 (reduced with 28 mM DTT, 0.5% Sarkosyl) was significantly reduced, consistent with B1 VNAR binding to conformational epitope(s).

FIG. 18.1: B1 forms a complex with ROR1 Ig domain by SEC. A, Overlayed SEC analysis (Superdex 200 Increase 10/300, GE Healthcare) of human ROR1 Ig domain with and without B1 his (orange and blue traces, respectively). B, SDS PAGE analysis of peak fractions.

FIG. 18.2: SEC analysis of ROR1-specific VNAR B1 binding to non-glycosylated version of ROR1 Ig domain (IgHis). Running Conditions: 20 mM Hepes, 150 mM NaCl, pH7.5. Arrow indicates peak selected for mass spectrometry analysis.

FIG. 18.3: Mass spectrometry analysis of additional peak formed when non-glycosylated version of ROR1 Ig domain (IgHis) and ROR1-specific VNAR B1 were analysed by SEC (FIG. 18.2). IgHis expected MW: 12,218.6 Da; IgHis observed MW: 12,217.9 Da. B1 expected MW: 12,506.8 Da; B1 observed MW: 12,506.0 Da. These data demonstrate that a complex between B1 and non-glycosylated IgHis has formed.

FIG. 18.4. Binding of VNAR domains D3 and P3A1 to ROR1 domains as assessed by SEC/SDS-PAGE analysis

FIG. 19a: SPR sensograms depicting binding of VNARs to hROR1 +/− previously captured B1 His6Myc VNAR. 2V monomer or dimer did not bind under any of these conditions.

FIG. 19b: Representative SPR sensograms depicting binding of i) ROR1 2A2 mAb, ii) UC961 based mAb, iii) P3A1, iv) B1, v) D3 and vi) E9 to hROR1 +/− previously captured P3A1 His6Myc dimer VNAR. No competition of binding to hROR1 was observed other than P3A1 self-competition (iii).

FIG. 20: B1 and P3A1 do not bind to selected linear ROR1 peptides by ELISA. Binding to human ROR1 is included as a positive control.

FIG. 21: B1, P3A1, D3 and D3-D3 do not bind to selected linear ROR1 peptides by ELISA. Binding to human ROR1 is included as a positive control.

FIG. 22: Competition ELISA experiments.

FIG. 23: Competition ELISA experiments.

FIG. 24: Binding of B1, P3A1, D3 monomer and D3-D3 dimer to different ROR1 domains.

FIG. 25: Schematic of BA11 aminoxy conjugation to benzaldehyde fluorescein.

FIG. 26: Schematic of BA11 thiol conjugation to maleimide fluorescein.

FIG. 27: Schematic of BA11 C-terminal cysteine derivative conjugation to maleimide fluorescein

FIG. 28: Examples of labels and payloads used for conjugation.

FIG. 29: Analysis of B1 MMAE conjugates. A, SDS PAGE analysis of B1 his myc derivatives and conjugates—lanes 1, B1 aminoxy; 2, B1 oxime MMAE; 3, B1 oxime vc MMAE; 4, B1 SH vc MMAE. B-F, electrospray mass spectra of B1 his myc derivatives and conjugates—B, B1 SH (expected mass; 14908.9 Da, observed mass 14908.4 Da); C, B1 SH vc MMAE (expected mass 16225.5 Da, observed mass 16225.5 Da); D, B1 aminoxy (expected mass 14937.4 Da, observed mass 14936.5 Da); E, B1 oxime MMAE (expected mass 16015.4 Da, observed mass 16016.7 Da); F, B1 oxime vc MMAE (expected mass 16334.4 Da, observed mass 16334.2 Da).

FIG. 30: Cell surface binding of B1-, P3A1- and 2V-hFc molecules vs the MMAE-conjugated versions in A549 (ROR1 hi) vs A427 (ROR1 low) lung cancer cell lines. VNAR hFc binding was detected using a PE-anti-human antibody (Jackson ImmunoResearch Labs/Stratech) and a ThermoFisher Attune NxT flow cytometer.

FIG. 31: Analysis of VNAR hFc conjugates. A&B, SDS PAGE analysis of VNAR hFc (S252C) proteins and conjugates (4-12% and 12% Bis Tris gel, respectively). Lanes 1, untreated protein, 2, refolded protein and 3, MMAE conjugate (+/− reduction with DTT). C&D, Example of mass spec analysis of deglycosylated, reduced VNAR hFc (S252C) fusion proteins before and after MMAE conjugation, respectively. Expected masses: unconjugated 38,997.8 Da and MMAE conjugate (DAR 2) 40,310.0 Da. E&F SDS PAGE analysis of VNAR hFc (S473C) protein conjugates. Lanes 3, MMAE conjugates and 4, AF488 conjugates (+/− reduction with DTT). G&H Mass spec analysis of deglycosylated, reduced B1- and P3A1 hFc (S473C) MMAE conjugates, respectively. Expected masses: B1 conjugate 40,170.5 Da and P3A1 conjugate 40,308.5 Da (DARs of 2) [* corresponds to MS artefact due to in source fragmentation]. I&J Mass spec analysis of deglycosylated, reduced B1- and P3A1 hFc (S473C) AF488 conjugates, respectively. Expected masses: B1 conjugate 39,552.4 Da and P3A1 conjugate 39,690.4 Da (DARs of 2).

FIG. 32: Schematic of VNAR hFc PBD dimer, amanitin and PNU conjugates.

FIG. 33: Cell viability following treatment with B1 mFc MMAE or 2V mFc-MMAE molecules (72 hr) in a panel of different human cancer cell lines. Cell Titre Glo reagent (Promega) was used to quantify ATP which correlates with the number of metabolically active cells in culture. IC50 values were determined using GraphPad Prism software.

FIG. 34: Cell viability following treatment with VNAR hFc PBD conjugates (96 hr) in 2 different human cancer cell lines (DU145 and Jeko-1). Cell Titre Glo reagent (Promega) was used to quantify ATP which correlates with the number of metabolically active cells in culture. IC50 values were determined using GraphPad Prism software. VNAR hFc conjugates were generated by reacting VNAR hIgG1 Fc(S252C) fusions with MA PEG4 va PBD (see FIG. 32).

FIG. 35: Cell viability following treatment with VNAR hFc PBD, SG3199 PBD and PNU (PEG4 vc PAB DMAE PNU159682) conjugates (96 hr) in 2 different human cancer cell lines (PA-1 and Kasumi-2). Cell Titre Glo reagent (Promega) was used to quantify ATP which correlates with the number of metabolically active cells in culture. IC50 values were determined using GraphPad Prism software. Whereby VNAR hFc conjugates were generated by reacting VNAR hIgG1 Fc(S252C) fusions with MA PEG4 va PBD, MA PEG8 va PAB SG3199, MA PEG4 vc PAB DMAE PNU 159682 (see FIG. 32).

FIG. 35b: PA-1 cells and Kasumi-2 cells were treated with IC80 concentrations of B1 hFc-SG3199, B1 hFc-PNU or 2VhFc non-binding controls in the presence or absence of increasing amounts of unconjugated B1 hFc or 2VhFc. Cell viability was assessed using Cell Titer Glo assay (Promega). Effects on cell viability following treatment with the protein-drug conjugate molecules were abrogated with increasing amounts of competing unconjugated B1 hFc but not with 2VhFc protein.

FIG. 36 QC data of B1-[(G4S)5]-D3 Alexa Fluor 488 conjugate. Top. SDS-PAGE analysis of the Alexa Fluor 488 VNAR conjugate. Visualisation using Coomassie Brilliant Blue or UV. SDS-PAGE carried out under reductive (+0.1 M DTT) or non-reductive (−0.1 M DTT) conditions. Bottom. Deconvoluted mass spectrum of the Alexa Fluor 488 VNAR conjugate. Observed mass (26286.8 Da) is consistent with the theoretical mass (29285.1 Da) expected for the selectively-labelled conjugate.

FIG. 37 QC data of P3A1-[(G4S)5]-BA11-[(G4S)5]-D3 Alexa Fluor 488 conjugate. Top. SDS-PAGE analysis of the Alexa Fluor 488 VNAR conjugate. Visualisation using Coomassie Brilliant Blue or UV. SDS-PAGE carried out under reductive (+0.1 M DTT) or non-reductive (−0.1 M DTT) conditions. Bottom. Deconvoluted mass spectrum of the Alexa Fluor 488 VNAR conjugate. Observed mass (42273.81 Da) is consistent with the theoretical mass (42,279.1) expected for the selectively-labelled conjugate.

FIG. 38 QC data of BA11-[PGVQPSPGGGGS]-B1 Alexa Fluor 488 conjugate. Top. SDS-PAGE analysis of the Alexa Fluor 488 VNAR conjugate. Visualisation using Coomassie Brilliant Blue or UV. SDS-PAGE carried out under reductive (+0.1 M DTT) or non-reductive (−0.1 M DTT) conditions. Bottom. Deconvoluted mass spectrum of the Alexa Fluor 488 VNAR conjugate. Observed mass (27821.12 Da) is consistent with the theoretical mass (27,819.99 Da) expected for the selectively-labelled conjugate.

In addition to the sequences mentioned the following sequences are expressly disclosed. Certain of these sequences relate to examples of molecules of the invention described herein:

| SEQ | |
|---|---|
| ID NO: | Sequence name |
| 115 | B1 Wobbe-G₄S-P3A1 CysHisMyc |
| 116 | P3A1 Wobbe-G₄S-B1 CysHisMyc |
| 117 | D3 Wobbe-G₄S-P3A1 CysHisMyc |
| 118 | P3A1 Wobbe-G₄S-D3 CysHisMyc |
| 119 | B1 Wobbe-G₄S-D3 CysHisMyc |
| 120 | B1 Wobbe-G₄S-BA11-Wobbe-G₄S-P3A1 |
| | CysHisMyc |
| 121 | BA11-Wobbe-G₄S-B1 -Wobbe-G₄S-P3A1 |
| | CysHisMyc |
| 122 | P3A1 Wobbe-G₄S-BA11-Wobbe-G₄S-B1 |
| | CysHisMyc |
| 123 | D3 Wobbe-G₄S-P3A1-Wobbe-G₄S-BA11 |
| | CysHisMyc |
| 124 | D3 Wobbe-G₄S-BA11 Wobbe-G₄S-P3A1 |
| | CysHisMyc |
| 125 | P3A1 Wobbe-G₄S-BA11-Wobbe-G₄S-D3 |
| | CysHisMyc |
| 126 | P3A1 Wobbe-G₄S-D3 Wobbe-G₄S-BA11 |
| | CysHisMyc |
| 127 | B1 Wobbe-G₄S-BA11-Wobbe-G₄S-D3 |
| | CysHisMyc |
| 128 | BA11-Wobbe-G₄S-B1-Wobbe-G₄S-D3 |
| | CysHisMyc |
| 129 | D3-Wobbe-G₄S-BA11-Wobbe-G₄S-B1 |
| | CysHisMyc |
| 130 | D3 Wobbe-G₄S-GM-P3A1 CysHisMyc |
| 131 | D3 Wobbe-G₄S-GM-P3A1-Wobbe-G₄S- |
| | GM-BA11 CysHisMyc |
| 132 | D3 Wobbe-G₄S-GM-BA11 Wobbe-G₄S- |
| | GM-P3A1 CysHisMyc |
| 133 | P3A1 Wobbe-G₄S-GM-D3 CysHisMyc |
| 134 | P3A1 Wobbe-G₄S-GM-BA11-Wobbe-G₄S- |
| | GM-D3 CysHisMyc |
| 135 | P3A1 Wobbe-G₄S-GM-D3 Wobbe-G₄S- |
| | GM-BA11 CysHisMyc |
| 136 | P3A1 Wobbe-G₄S-BA11-Wobbe-G₄S- |
| | P3A1 CysHisMyc |
| 137 | BA11 Wobbe-G₄S-P3A1-Wobbe-G₄S- |
| | P3A1-CysHisMyc |
| 138 | 2V-Wobbe-G₄S-2V CysHisMyc |
| 139 | 2V-Wobbe-G₄S-BA11-Wobbe-G₄S-2V |
| | CysHisMyc |
| 140 | BA11-Wobbe-G₄S-B1 CysHisMyc |
| 141 | BA11-Wobbe-G₄S-GM-B1 CysHisMyc |
| 142 | BA11-Wobbe-G₄S-B1 HisMyc |
| 143 | BA11-Wobbe-G₄S-GM-B1 HisMyc |
| 144 | B1-D3 HisMyc |
| 145 | D3-P3A1 HisMyc |
| 146 | P3A1-D3 HisMyc |
| 147 | D3-B1 CysHisMyc |
| 148 | B1-D3 CysHisMyc |
| 149 | D3-P3A1 CysHisMyc |
| 150 | P3A1-D3 CysHisMyc |
| 151 | B1-BA11-P3A1 HisMyc |
| 152 | B1-BA11-D3 HisMyc |
| 153 | D3-BA11-B1 CysHisMyc |
| 154 | D3-BA11-B1 HisMyc |
| 155 | D3-BA11-P3A1 HisMyc |
| 156 | P3A1-BA11-D3 CysHisMyc |
| 157 | P3A1-BA11-D3 HisMyc |
| 158 | P3A1-B1 HisMyc |
| 159 | P3A1-D3 His |
| 160 | P3A1-B1 SAPSA |
| 161 | 2V-BA11CysHisMyc |
| 162 | 2V-BA11HisMyc |
| 163 | BA11-2V HisMyc |
| 164 | D3-D3-BA11 HisMyc |
| 165 | D3-D3-BA11 CysHisMyc |
| 166 | E9-BA11-E9 HisMyc |
| 167 | D3-BA11-D3 HisMyc |
| 168 | P3A1-BA11-P3A1 HisMyc |
| 169 | BA11-B1 HisMyc |
| 170 | B1-Hinge-BA11 CysHisMyc |
| 171 | BA11-Hinge-B1 CysHisMyc |
| 172 | BA11 Helical B1 CysHisMyc |
| 173 | BA11-DoubleHinge-B1 CysHisMyc |
| 174 | B1-Hinge-BA11 CysHisMyc |
| 175 | BA11-Hinge-B1 CysHisMyc |
| 176 | BA11 Helical B1 CysHisMyc |
| 177 | BA11-DoubleHinge-B1 CysHisMyc |
| 178 | P3A1-(G₄S)₃-B1 HisMyc |
| 179 | P3A1-(G₄S)₇-B1 HisMyc |
| 180 | BA11-(G4S)3-B1 CysHisMyc |
| 181 | 2V |
| 182 | B1 his myc |
| 183 | B1 [QASGA] his |
| 184 | B1 [QASGA] |
| 185 | B1 [QACGA] his |
| 186 | B1 [QACKA] his |
| 187 | B1 SAPSA |
| 188 | B1 Hinge |
| 189 | B1 his myc(HEK293) |
| 190 | P3A1 his myc |
| 191 | P3A1 his |
| 192 | P3A1 |
| 193 | D3 his myc |
| 194 | E9 his myc |
| 195 | E7 his myc |
| 196 | CPF7 his myc |
| 197 | BA11 |
| 198 | B1G1 |
| 199 | B1G2 |
| 200 | B1V1 |
| 201 | B1V5 |
| 202 | B1V7 |
| 203 | D3V1 |
| 204 | D3V2 |
| 205 | D3V3 |
| 206 | D3 ELV1 |
| 207 | D3 ELV2 |
| 208 | D3 ELV3 |
| 209 | D3 ELV4 |
| 210 | D3 ELV5 |
| 211 | P3A1-P3A1 his myc |
| 212 | P3A1-P3A1 his |
| 213 | P3A1-P3A1 [SAPSA] |
| 214 | D3-D3 his myc |
| 215 | D3-D3 his |
| 216 | CPF7-CPF7 his myc |
| 217 | D3-D3 QACGA |
| 218 | D3-D3 QACGA His |
| 219 | P3A1V1 dimer |
| 220 | P3A1G1 dimer |
| 221 | P3A1G2 dimer |
| 222 | D3V1 dimer |
| 223 | D3V2 dimer |
| 224 | D3V3 dimer |
| 225 | B1G1-B1G1 HisMyc |
| 226 | B1G2-B1G2 HisMyc |
| 227 | B1 mFc |
| 228 | 2VmFc |
| 229 | B1 hFc (S252C) |
| 230 | B1G1 hFc (S252C) |
| 231 | B1G2 hFc (S252C) |
| 232 | B1 hFc (S473C) |
| 233 | P3A1 hFc (S252C) |
| 234 | hFc (S252C) P3A1 |
| 235 | P3A1 hFc(S473C) |
| 236 | D3 hFc (S252C) |
| 237 | hFc(S252C) D3 |
| 238 | D3D3 hFc (S252C) |
| 239 | hFc (S252C) D3D3 |
| 240 | E9 hFc (S252C) |
| 241 | 2V hFc (S252C) |
| 242 | 2V hFc (S473C) |
| 243 | human ROR1-Fc (HEH293) |
| 244 | human ROR1-Fc (CHO) |
| 245 | human ROR1 (Ig domain)-Fc (CHO) |
| 246 | human ROR1 (FZ domain)-Fc (CHO) |
| 247 | human ROR1 (KR domain)-Fc (CHO) |
| 248 | human ROR1 (IgFZ domains)-Fc (CHO) |
| 249 | human ROR1 (FZKR domains)-Fc (CHO) |
| 250 | human ROR2-Fc (CHO) |
| 251 | mouse ROR1-Fc (CHO) |
| 252 | rat ROR1-Fc (CHO) |
| 253 | human ROR1-His (HEK293 |
| 254 | human ROR1-His (CHO) |
| 255 | human ROR1 (Ig domain)-His |
| 256 | mouse ROR1-His (HEK293) |
| 257 | B1 × CD3 [G4S] his |
| 258 | B1 × CD3 [G4S]3 his |
| 259 | P3A1 × CD3 [G4S] his |
| 260 | P3A1 × CD3 [G4S]4 his |
| 261 | P3A1-P3A1 × CD3 [G4S] his |
| 262 | P3A1-P3A1 × CD3 [G4S]5 his |
| 263 | 2V × CD3 [G4S] his |
| 264 | 2V × CD3 [G4S]3 his |
| 265 | P3A1-[W]-B1-[G4S]-CD3 his |
| 266 | P3A1-[W]-B1-[G4S]3-CD3 his |
| 267 | P3A1-[WGM]-D3-[G4S]-CD3 his |
| 268 | P3A1-[WGM]-D3-[G4S]3-CD3 his |
| 269 | P3A1-[G4S]5-D3-[G4S]-CD3 his |
| 270 | P3A1-[G4S]5-D3-[G4S]3-CD3 his |

### DETAILED DESCRIPTION

The present invention generally relates to specific antigen binding molecules. Specifically, the invention provides immunoglobulin-like shark variable novel antigen receptors (VNARs) specific for receptor tyrosine kinase-like orphan receptor 1 (ROR1) and associated fusion proteins, chimeric antigen receptors, conjugates, and nucleic acids, as well as accompanying methods. The ROR1-specific VNAR domains are described herein as ROR1-specific antigen binding molecules.

The Novel or New antigen receptor (IgNAR) is an approximately 160 kDa homodimeric protein found in the sera of cartilaginous fish (Greenberg A. S., et al., Nature, 1995. 374(6518): p. 168-173, Dooley, H., et al, Mol. Immunol, 2003. 40(1): p. 25-33; Müller, M. R., et al., mAbs, 2012. 4(6): p. 673-685)). Each molecule consists of a single N-terminal variable domain (VNAR) and five constant domains (CNAR). The IgNAR domains are members of the immunoglobulin-superfamily. The VNAR is a tightly folded domain with structural and some sequence similarities to the immunoglobulin and T-cell receptor Variable domains and to cell adhesion molecules and is termed the VNAR by analogy to the N Variable terminal domain of the classical immunoglobulins and T Cell receptors. The VNAR shares limited sequence homology to immunoglobulins, for example 25-30% similarity between VNAR and human light chain sequences (Dooley, H. and Flajnik, M. F., Eur. J. Immunol., 2005. 35(3): p. 936-945).

Kovaleva M. et al Expert Opin. Biol. Ther. 2014. 14(10): p. 1527-1539 and Zielonka S. et al mAbs 2015. 7(1): p. 15-25 provided summaries of the structural characterization and generation of the VNARs, which are hereby incorporated by reference.

The VNAR does not appear to have evolved from a classical immunoglobulin antibody ancestor. The distinct structural features of VNARs are the truncation of the sequences equivalent to the CDR2 loop present in conventional immunoglobulin variable domains and the lack of the hydrophobic VH/VL interface residues which would normally allow association with a light chain domain, which is not present in the IgNAR structure. Furthermore, unlike classical immunoglobulins some VNAR subtypes include extra cysteine residues in the CDR regions that are observed to form disulphide bridges in addition to the canonical Immunoglobulin superfamily bridge between the Cysteines in the Framework 1 and 3 regions N terminally adjacent to CDRs 1 and 3.

To date, there are three defined types of shark IgNAR known as I, II and III. These have been categorized based on the position of non-canonical cysteine residues which are under strong selective pressure and are therefore rarely replaced.

All three types have the classical immunoglobulin canonical cysteines at positions 35 and 107 (numbering as in Kabat, E. A. et al. Sequences of proteins of immunological interest. 5th ed. 1991, Bethesda: US Dept. of Health and Human Services, PHS, NIH) that stabilize the standard immunoglobulin fold, together with an invariant tryptophan at position 36. There is no defined CDR2 as such, but regions of sequence variation that compare more closely to TCR HV2 and HV4 have been defined in framework 2 and 3 respectively. Type I has germline encoded cysteine residues in framework 2 and framework 4 and an even number of additional cysteines within CDR3. Crystal structure studies of a Type I IgNAR isolated against and in complex with lysozyme enabled the contribution of these cysteine residues to be determined. Both the framework 2 and 4 cysteines form disulphide bridges with those in CDR3 forming a tightly packed structure within which the CDR3 loop is held tightly down towards the HV2 region. To date Type I IgNARs have only been identified in nurse sharks—all other elasmobranchs, including members of the same order have only Type II or variations of this type.

Type II IgNAR are defined as having a cysteine residue in CDR1 and CDR3 which form intramolecular disulphide bonds that hold these two regions in close proximity, resulting in a protruding CDR3 (FIG. 2) that is conducive to binding pockets or grooves. Type I sequences typically have longer CDR3s than type II with an average of 21 and 15 residues respectively. This is believed to be due to a strong selective pressure for two or more cysteine residues in Type I CDR3 to associate with their framework 2 and 4 counterparts. Studies into the accumulation of somatic mutations show that there are a greater number of mutations in CDR1 of type II than type I, whereas HV2 regions of Type I show greater sequence variation than Type II. This evidence correlates well with the determined positioning of these regions within the antigen binding sites.

A third IgNAR type known as Type III has been identified in neonates. This member of the IgNAR family lacks diversity within CDR3 due to the germline fusion of the D1 and D2 regions (which form CDR3) with the V-gene. Almost all known clones have a CDR3 length of 15 residues with little or no sequence diversity.

Another structural type of VNAR, termed type (IIb or IV), has only two canonical cysteine residues (in framework 1 and framework 3b regions). So far, this type has been found primarily in dogfish sharks (Liu, J. L., et al. Mol. Immunol. 2007. 44(7): p. 1775-1783; Kovalenko O. V., et al. J Biol Chem. 2013. 288(24): p. 17408-19) and was also isolated from semisynthetic V-NAR libraries derived from wobbegong sharks (Streltsov, V. A. et al. (2004) Proc. Natl. Acad. Sci. U.S.A. 101(34): p. 12444-12449).

It has been shown however specific VNARs isolated from synthetic libraries formed from the VNAR sequences can bind with high affinity to other proteins (Shao C. Y. et al. Mol Immunol. 2007. 44(4): p. 656-65; WO2014/173959) and that the IgNAR is part of the adaptive immune system as cartilaginous fish can be immunized with antigen and responsive IgNARs obtained that bind to the antigen (Dooley, H., et al, Mol. Immunol, 2003. 40(1): p. 25-33; WO2003/014161). It has been shown that the IgNAR has a mechanism for combinatorial joining of V like sequences with D and J sequences similar to that of immunoglobulins and the T cell receptor (summarized by Zielonka S. et al mAbs 2015. 7(1): p. 15-25).

The VNAR binding surface, unlike the variable domains in other natural immunoglobulins, derives from four regions of diversity: CDR1, HV2, HV4 and CDR3 (see also Stanfield, R. L., et al, Science, 2004. 305(5691): p. 1770-1773; Streltsov, V. A., et al, Protein Sci., 2005. 14(11): p. 2901-2909; Stanfield, R. L., et al., J Mol. Biol., 2007. 367(2): p. 358-372), joined by intervening framework sequences in the order: FW1-CDR1-FW2-HV2-FW3a-HV4-FW3b-CDR3-FW4. The combination of a lack of a natural light chain partner and lack of CDR2 make VNARs the smallest naturally occurring binding domains in the vertebrate kingdom.

The IgNAR shares some incidental features with the heavy chain only immunoglobulin (HCAb) found in camelidae (camels, dromedaries and llamas, Hamers-Casterman, C. et al. Nature, 1993. 363, 446-448; Wesolowski, J., et al., Med Microbiol Immunol, 2009. 198(3): p. 157-74) Unlike the IgNAR the HCAb is clearly derived from the immunoglobulin family and shares significant sequence homology to standard immunogloblulins. Importantly one key distinction of VNARs is that the molecule has not had at any point in its evolution a partner light chain, unlike classical immunoglobulins or the HCAbs. Flajnik M. F. et al PLoS Biol 2011. 9(8): el 001120 and Zielonka S. et al mAbs 2015. 7(1): p. 15-25 have commented on the similarities and differences between, and the possible and distinct evolutionary origins of, the VNAR and the immunoglobulin-derived VHH single binding domain from the camelids.

Although antibodies to ROR1 have been reported in the literature, the high sequence identity between the extracellular domain of human, mouse and rat ROR1 and between human ROR1 and ROR2 family members means generating high affinity hROR1-specific binding agents is not trivial. Additionally, the large size of antibodies compromises their ability to penetrate into solid tumours and render regions of target proteins inaccessible due to steric factors, which can be particularly acute for cell-surface proteins where oligomerisation or receptor clustering is observed.

As a result there is a need in the art for improved anti-ROR1 binding protein agents with different functional or physical characteristics or properties to antibodies and the development of therapeutics and diagnostic agents for malignancies associated with ROR1 expression. The present invention provides such agents in the form of the ROR1-specific antigen binding molecules described herein.

The presently-described ROR1-specific antigen binding molecules have been shown to bind to both human and murine ROR1. Furthermore, the ROR1-specific antigen binding molecules of the present invention bind to deglycosylated forms of ROR1 and do not bind to a number of linear peptides associated with anti-ROR1 antibodies described in the prior art. The presently-described ROR1-specific antigen binding molecules are therefore thought to bind to novel epitopes in the ROR1 sequence.

Binding of the ROR1-specific antigen binding molecules of the invention to cancer cell lines, as well as internalisation, have been demonstrated. This confirms the potential for the use of such molecules in the treatment of cancers, specifically cancers which express ROR1.

Various forms of the ROR1-specific antigen binding molecules are described, including fusion proteins of several types. Fusion proteins including an immunoglobulin Fc region are described, as well as both homo and heterodimers. Fusion of proteins to an Fc domain can improve protein solubility and stability, markedly increase plasma half-life and improve overall therapeutic effectiveness.

The present inventors have also, for the first time, created VNAR molecules conjugated to a variety of moieties and payloads. The present invention therefore also provides chemically conjugated VNARs. More specifically, ROR1-specific antigen molecules in several conjugated formats are provided.

### Definitions

An antigen specific binding molecule of the invention comprises amino acid sequence derived from a synthetic library of VNAR molecules, or from libraries derived from the immunization of a cartilaginous fish. The terms VNAR, IgNAR and NAR may be used interchangeably also.

Amino acids are represented herein as either a single letter code or as the three letter code or both.

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “Complementarity Determining Regions” or CDRs (i.e., CDR1 and CDR3) refers to the amino acid residues of a VNAR domain the presence of which are typically involved in antigen binding. Each VNAR typically has two CDR regions identified as CDR1 and CDR3. Additionally, each VNAR domain comprises amino acids from a “hypervariable loop” (HV), which may also be involved in antigen binding. In some instances, a complementarity determining region can include amino acids from both a CDR region and a hypervariable loop. In other instances, antigen binding may only involve residues from a single CDR or HV. According to the generally accepted nomenclature for VNAR molecules, a CDR2 region is not present.

“Framework regions” (FVV) are those VNAR residues other than the CDR residues. Each VNAR typically has five framework regions identified as FW1, FW2, FW3a, FW3b and FW4.

The boundaries between FW, CDR and HV regions in VNARs are not intended to be fixed and accordingly some variation in the lengths and compositions of these regions is to be expected. This will be understood by those skilled in the art, particularly with reference to work that have been carried out in analyzing these regions. (Anderson et al., PLoS ONE (2016) 11 (8); Lui et al., Mol Immun (2014) 59, 194-199; Zielonka et al., Mar Biotechnol (2015). 17, (4) 386-392; Fennell et al., J Mol Biol (2010) 400. 155-170; Kovalenko et al., J Biol Chem (2013) 288. 17408-17419; Dooley et al., (2006) PNAS 103 (6). 1846-1851). The molecules of the present invention, although defined by reference to FW, CDR and HV regions herein, are not limited to these strict definitions. Variation in line with the understanding in the art as the structure of the VNAR domain is therefore expressly contemplated herein.

A “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein.

A codon set is typically represented by 3 capital letters in italics, e.g. NNK, NNS, XYZ, DVK etc. A “non-random codon set” therefore refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al.; J. Mol. Biol. (1999), 296, 57-86); Garrard & Henner, Gene (1993), 128, 103). Such sets of oligonucleotides having certain codon sets can be synthesized using commercial nucleic acid synthesizers (available from, for example, Applied Biosystems, Foster City, CA), or can be obtained commercially (for example, from Life Technologies, Rockville, MD). A set of oligonucleotides synthesized having a particular codon set will typically include a plurality of oligonucleotides with different sequences, the differences established by the codon set within the overall sequence. Oligonucleotides used according to the present invention have sequences that allow for hybridization to a VNAR nucleic acid template and also may where convenient include restriction enzyme sites.

“Cell”, “cell line”, and “cell culture” are used interchangeably (unless the context indicates otherwise) and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, etc. Eukaryotic cells use control sequences such as promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell.

The “detection limit” for a chemical entity in a particular assay is the minimum concentration of that entity which can be detected above the background level for that assay. For example, in the phage ELISA, the “detection limit” for a particular phage displaying a particular antigen binding fragment is the phage concentration at which the particular phage produces an ELISA signal above that produced by a control phage not displaying the antigen binding fragment.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

The term “fusion protein” in this text means, in general terms, one or more proteins joined together by chemical means, including hydrogen bonds or salt bridges, or by peptide bonds through protein synthesis or both. Typically fusion proteins will be prepared by DNA recombination techniques and may be referred to herein as recombinant fusion proteins.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from an allogenic or xenogenic source. The DNA may, optionally, include marker or selection genes, for example, antibiotic resistance genes, temperature resistance genes, etc.

A “highly diverse position” refers to a position of an amino acid located in the variable regions of the light and heavy chains that have a number of different amino acid represented at the position when the amino acid sequences of known and/or naturally occurring antibodies or antigen binding fragments are compared. The highly diverse positions are typically in the CDR or HV regions.

“Identity” describes the relationship between two or more polypeptide sequences or two or more polynucleotide sequences, as determined by comparing the sequences. Identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic acids Research, 12, 387 (1984), BLASTP, BLASTN, and FASTA (Atschul et al., J. Molec. Biol. (1990) 215, 403).

Preferably, the amino acid sequence of the protein has at least 45% identity, using the default parameters of the BLAST computer program (Atschul et al., J. Mol. Biol. (1990) 215, 403-410) provided by HGMP (Human Genome Mapping Project), at the amino acid level, to the amino acid sequences disclosed herein.

More preferably, the protein sequence may have at least 45%, 46%, 47%, 48%, 49%, 50%, 55%, 60%, 65%, 66%, 67%, 68%, 69%, 70%, 75%, 80%, 85%, 90% and still more preferably 95% (still more preferably at least 96%, 97%, 98% or 99%) identity, at the nucleic acid or amino acid level, to the amino acid sequences as shown herein.

The protein may also comprise a sequence which has at least 45%, 46%, 47%, 48%, 49%, 50%, 50%, 55%, 60%, 65%, 66%, 67%, 68%, 69%, 70%, 75%, 80%, 85%, 90%, 95%, 96%, 97%, 98%, or 99% identity with a sequence disclosed herein, using the default parameters of the BLAST computer program provided by HGMP, thereto

A “library” refers to a plurality of VNARs or VNAR fragment sequences (for example, polypeptides of the invention), or the nucleic acids that encode these sequences, the sequences being different in the combination of variant amino acids that are introduced into these sequences according to the methods of the invention.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation or by silica purification. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step.

A “mutation” is a deletion, insertion, or substitution of a nucleotide(s) relative to a reference nucleotide sequence, such as a wild type sequence.

“Natural” or “naturally occurring” VNARs, refers to VNARs identified from a non-synthetic source, for example, from a tissue source obtained ex vivo, or from the serum of an animal of the Elasmobranchii subclass. These VNARs can include VNARs generated in any type of immune response, either natural or otherwise induced. Natural VNARs include the amino acid sequences, and the nucleotide sequences that constitute or encode these antibodies. As used herein, natural VNARs are different than “synthetic VNARs”, synthetic VNARs referring to VNAR sequences that have been changed from a source or template sequence, for example, by the replacement, deletion, or addition, of an amino acid, or more than one amino acid, at a certain position with a different amino acid, the different amino acid providing an antibody sequence different from the source antibody sequence.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promotor or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contingent and in reading frame. However, enhancers do not have to be contiguous. Linking is accomplished by ligation at convenient restriction sites. If such sites do not exist, the synthetic oligonucleotide adapters or linkers are used in accord with conventional practice.

The term “protein” means, in general terms, a plurality of amino acid residues joined together by peptide bonds. It is used interchangeably and means the same as peptide, oligopeptide, oligomer or polypeptide, and includes glycoproteins and derivatives thereof. The term “protein” is also intended to include fragments, analogues, variants and derivatives of a protein wherein the fragment, analogue, variant or derivative retains essentially the same biological activity or function as a reference protein. Examples of protein analogues and derivatives include peptide nucleic acids, and DARPins (Designed Ankyrin Repeat Proteins).

A fragment, analogue, variant or derivative of the protein may be at least 25 preferably 30 or 40, or up to 50 or 100, or 60 to 120 amino acids long, depending on the length of the original protein sequence from which it is derived. A length of 90 to 120, 100 to 110 amino acids may be convenient in some instances.

The fragment, derivative, variant or analogue of the protein may be (i) one in which one or more of the amino acid residues are substituted with a conserved or non-conserved amino acid residue (preferably, a conserved amino acid residue) and such substituted amino acid residue may or may not be one encoded by the genetic code, or (ii) one in which one or more of the amino acid residues includes a substituent group, or (iii) one in which the additional amino acids are fused to the mature polypeptide, such as a leader or auxiliary sequence which is employed for purification of the polypeptide. Such fragments, derivatives, variants and analogues are deemed to be within the scope of those skilled in the art from the teachings herein.

“Oligonucleotides” are short-length, single- or double-stranded polydeoxynucleotides that are chemically synthesized by known methods (such as phosphotriester, phosphite, or phosphoramidite chemistry, using solid-phase techniques). Further methods include the polymerase chain reaction (PCR) used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation. DNA is “purified” when the DNA is separated from non-nucleic acid impurities (which may be polar, non-polar, ionic, etc.).

A “source” or “template” VNAR, as used herein, refers to a VNAR or VNAR antigen binding fragment whose antigen binding sequence serves as the template sequence upon which diversification according to the criteria described herein is performed. An antigen binding sequence generally includes within a VNAR preferably at least one CDR, preferably including framework regions.

A “transcription regulatory element” will contain one or more of the following components: an enhancer element, a promoter, an operator sequence, a repressor gene, and a transcription termination sequence.

“Transformation” means a process whereby a cell takes up DNA and becomes a “transformant”. The DNA uptake may be permanent or transient. A “transformant” is a cell which has taken up and maintained DNA as evidenced by the expression of a phenotype associated with the DNA (e.g., antibiotic resistance conferred by a protein encoded by the DNA).

A “variant” or “mutant” of a starting or reference polypeptide (for example, a source VNAR or a CDR thereof), such as a fusion protein (polypeptide) or a heterologous polypeptide (heterologous to a phage), is a polypeptide that (1) has an amino acid sequence different from that of the starting or reference polypeptide and (2) was derived from the starting or reference polypeptide through either natural or artificial mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a nonrandom codon set that encodes a sequence with a variant amino acid (with respect to the amino acid found at the corresponding position in a source VNAR or antigen binding fragment) would be a variant polypeptide with respect to a source VNAR or antigen binding fragment. Thus, a variant CDR refers to a CDR comprising a variant sequence with respect to a starting or reference polypeptide sequence (such as that of a source VNAR or antigen binding fragment). A variant amino acid, in this context, refers to an amino acid different from the amino acid at the corresponding position in a starting or reference polypeptide sequence (such as that of a source VNAR or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

A “wild-type” or “reference” sequence or the sequence of a “wild-type” or “reference” protein/polypeptide, such as a coat protein, or a CDR of a source VNAR, may be the reference sequence from which variant polypeptides are derived through the introduction of mutations. In general, the “wild-type” sequence for a given protein is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man induced means. The products of such processes are “variant” or “mutant” forms of the original “wild-type” protein or gene.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of an antigen-specific binding protein, such as a monoclonal antibody or VNAR, onto a T cell, thereby allowing a large number of specific T cells to be generated, for example, for use in adoptive cell therapy. CARs may direct the specificity of the cell to a tumour associated antigen, for example. CARs may comprise an intracellular activation domain, a transmembrane domain, and an extracellular domain comprising a tumour associated antigen binding region. In particular aspects, CARs comprise fusions of single-chain variable fragments (scFv) derived from monoclonal antibodies fused to CD3-zeta transmembrane and endodomains. In other particular aspects, CARs comprise fusions of the VNAR domains described herein with CD3-zeta transmembrane and endodomains. The specificity of other CAR designs may be derived from ligands of receptors (e.g., peptides) or from pattern-recognition receptors, such as Dectins. In particular embodiments, one can target malignant B cells by redirecting the specificity of T cells by using a CAR specific for the B-lineage molecule, CD 19. In certain cases, the spacing of the antigen-recognition domain can be modified to reduce activation-induced cell death. In certain cases, CARs comprise domains for additional co-stimulatory signalling, such as CD3-zeta, FcR, CD27, CD28, CD 137, DAP 10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “conjugation” as used herein may refer to any method of chemically linking two or more chemical moieties. Typically, conjugation will be via covalent bond. In the context of the present invention, at least one of the chemical moieties will be a polypeptide and in some cases the conjugation will involve two or more polypeptides, one or more of which may be generated by recombinant DNA technology. A number of systems for conjugating polypeptides are known in the art. For example, conjugation can be achieved through a lysine residue present in the polypeptide molecule using N-hydroxy-succinimide or through a cysteine residue present in the polypeptide molecule using maleimidobenzoyl sulfosuccinimide ester. In some embodiments, conjugation occurs through a short-acting, degradable linkage including, but not limited to, physiologically cleavable linkages including ester, carbonate ester, carbamate, sulfate, phosphate, acyloxyalkyl ether, acetal, and ketal, hydrazone, oxime and disulphide linkages. In some embodiments linkers that are cleavable by intracellular or extracellular enzymes, such as cathepsin family members, cleavable under reducing conditions or acidic pH are incorporated to enable releases of conjugated moieties from the polypeptide or protein to which it is conjugated.

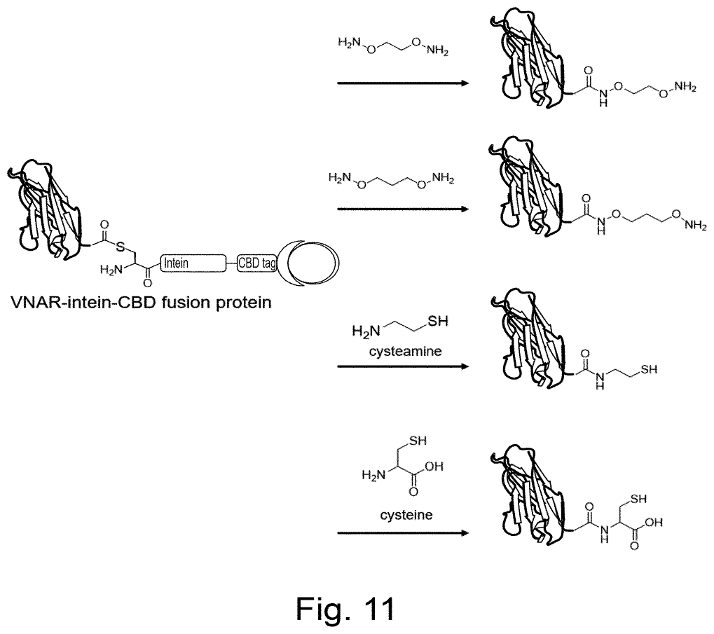

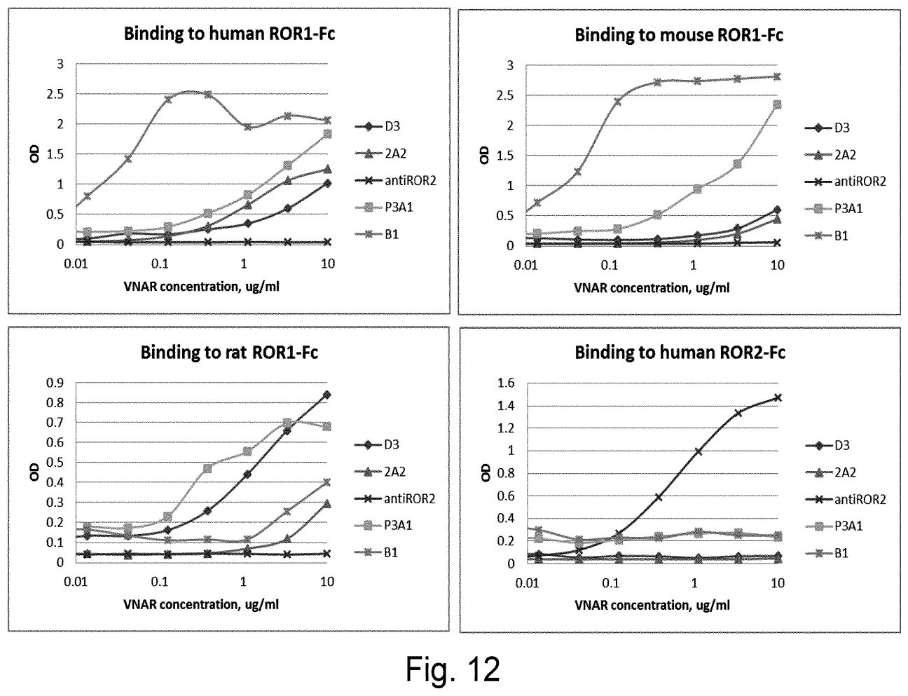

A particularly preferred method of conjugation is the use of intein-based technology (US2006247417) Briefly, the protein of interest is expressed as an N terminal fusion of an engineered intein domain (Muir 2006 Nature 442, 517-518). Subsequent N to S acyl shift at the protein-intein union results in a thioester linked intermediate that can be chemically cleaved with bis-aminoxy agents or amino-thiols to give the desired protein C-terminal aminoxy or thiol derivative, respectively (FIG. 11). These C-terminal aminoxy and thiol derivatives can be reacted with aldehyde/ketone and maleimide functionalised moieties, respectively, in a chemoselective fashion to give the site-specific C-terminally modified protein (FIGS. 25-27).

In another preferred method of conjugation the VNARs are directly expressed with an additional cysteine at or near the C-terminal region of the VNAR or incorporated within a short C-terminal tag sequence enabling conjugation with thiol reactive payloads such as maleimide functionalised moieties.

Conjugation as referred to herein is also intended to encompass the use of a linker moiety, which may impart a number of useful properties. Linker moieties include, but are not limited to, peptide sequences such as poly-glycine, gly-ser, val-cit or val-ala. In certain cases, the linker moiety may be selected such that it is cleavable under certain conditions, for example via the use of enzymes, nucleophilic/basic reagents, reducing agents, photo-irradiation, electrophilic/acidic reagents, organometallic and metal reagents, or oxidizing reagents, or the linker may be specifically selected to resist cleavage under such conditions.

Polypeptides may be conjugated to a variety of functional moieties in order to achieve a number of goals. Examples of functional moieties include, but are not limited to, polymers such as polyethylene glycol in order to reduce immunogenicity and antigenicity or to improve solubility. Further non-limiting examples include the conjugation of a polypeptide to a therapeutic agent or a cytotoxic agent.

The term “detectable label” is used herein to specify that an entity can be visualized or otherwise detected by spectroscopic, photochemical, biochemical, immunochemical, electrical, optical, chemical or other means. The detectable label may be selected such that it generates a signal which can be measured and whose intensity is proportional to the amount of bound entity. A wide variety of systems for labelling and/or detecting proteins and peptides are known in the art. A label may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionuclides, fluorophores, chemiluminescent agents, microparticles, enzymes, colorimetric labels, magnetic labels, haptens, molecular beacons, and aptamer beacons.

Methods of killing or inhibiting the growth of a cells expressing ROR1 in vitro or in a patient are contemplated herein, In general, them “killing” as used herein in the context of cells means causing a cell death. This may be achieved by a number of mechanisms, such as necrosis or other cells injury, or the induction of apoptosis. The phrases “inhibiting the growth” or “inhibiting proliferation” when used herein are intended to encompass the prevention of cell development, more specifically the prevention of cell division.

The present invention will be further understood by reference to the following examples.

### EXAMPLES

### Example 1—Generation of Specific Anti-ROR1 VNAR Sequences

Specific VNAR Sequences from Synthetic Library

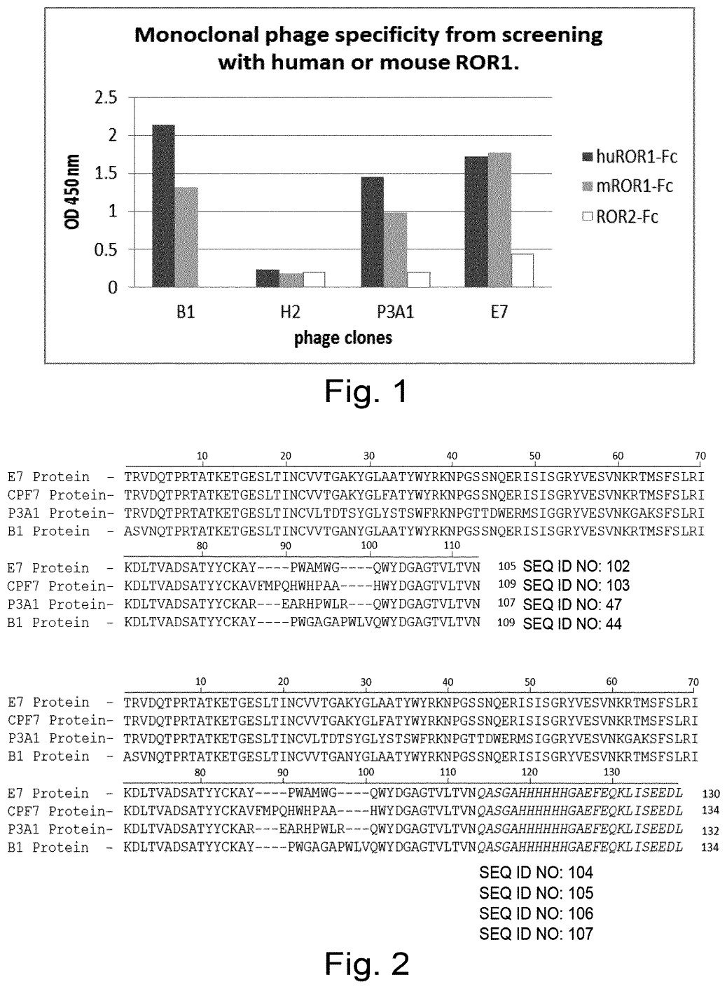

Two selection campaigns were adopted for screening a VNAR synthetic domain library (WO2014173959) for specific ROR1 binders. The first campaign made use of human ROR1 antigen and the second used mouse ROR1 antigen. Both recombinant ROR1 proteins were biotinylated as per manufacturer's instructions (Thermo Scientific Sulfo-NHS-LC-Biotin protocol, Cat N 21327) to aid the antigen presentation and selection process. VNAR domains were isolated after 3 rounds of selection using these biotinylated ROR1 antigens immobilised on streptavidin-coated beads. Post selection and following the screening of individual clones, 70% of monoclonal phage displaying VNAR domains (selected against human ROR1 protein) were found to be specific to human and mouse ROR1, but not a closely related ROR2 protein (the lead clones from this selection were called B1—40% and E7—30% (FIG. 1). Similarly, 45% of monoclonal phage selected with mouse ROR1 were specific to human and mouse ROR1, but not ROR2 (lead clone from this selection was called P3A1, FIG. 1). Another specific clone obtained from mouse ROR1 screening was CPF7 which was present as a single sequence out of 200 screened clones.

The sequences obtained from screening with human ROR1 are B1 and E7, and from screening with mouse ROR1 is P3A1 and CPF7. (FIG. 2)

Specific VNAR Sequences from Immunised Libraries

### Libraries Construction.

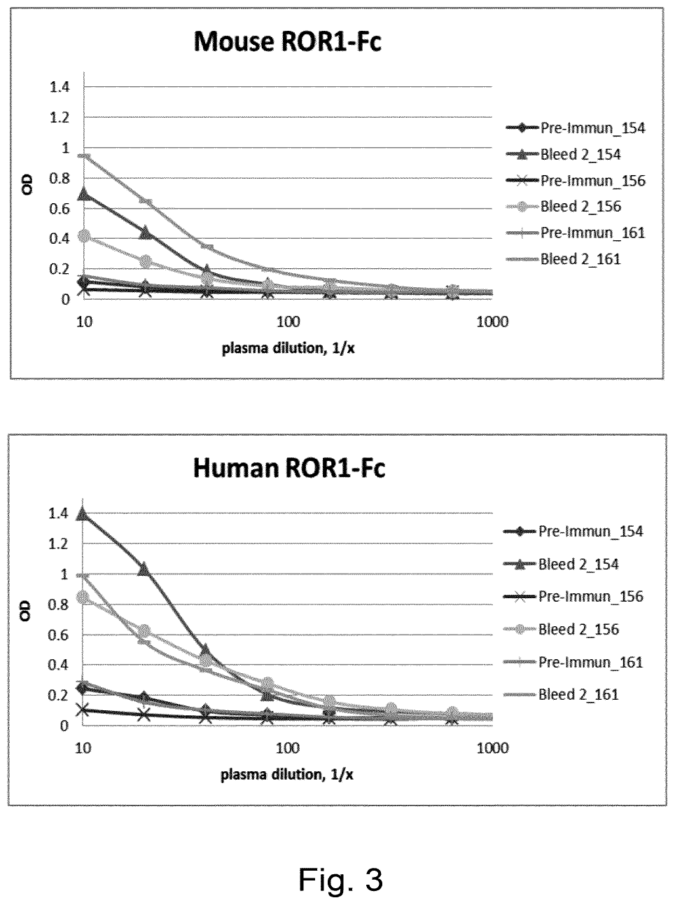

Three spiny dogfish were immunised with extracellular domain of recombinant human ROR1 protein and a target-specific IgNAR immune response was monitored through the analysis of post-immunised sera as described in Müller M. R. et al. Generation and Isolation of Target-Specific Single-Domain Antibodies from Shark Immune Repertoires, Humana Press 2012. Sera samples pre- and post-immunisation were taken from animals and tested for antigen binding in ELISA. An IgNAR titre increase, specific for human ROR1, was observed after 16 weeks in all animals (FIG. 3). The specificity of post-immune sera to mouse ROR1 was also observed indicating the presence in immunised animals of species cross-reactive ROR1 specific IgNAR binders (FIG. 3).

The VNAR repertoire (binding sites of IgNAR) was amplified from dogfish blood using specific PCR primers and cloned into a phage display vector, which contained an in-frame coat protein pIII of the bacteriophage M13 gene as described in Müller M. R. et al. Generation and Isolation of Target-Specific Single-Domain Antibodies from Shark Immune Repertoires, Humana Press 2012. The library sizes were calculated and are shown in Table 1:

TABLE 1

| Fish # | Library | Size (unique transformants) |
|---|---|---|
| 154 | ELSI 5 | 6 × 107 |
| 156 | ELSI 6 | 1.7 × 107 |
| 161 | ELSI 7 | 2 × 107 |

### Screening of the Immunised Libraries for Antigen Specific VNAR Sequences.

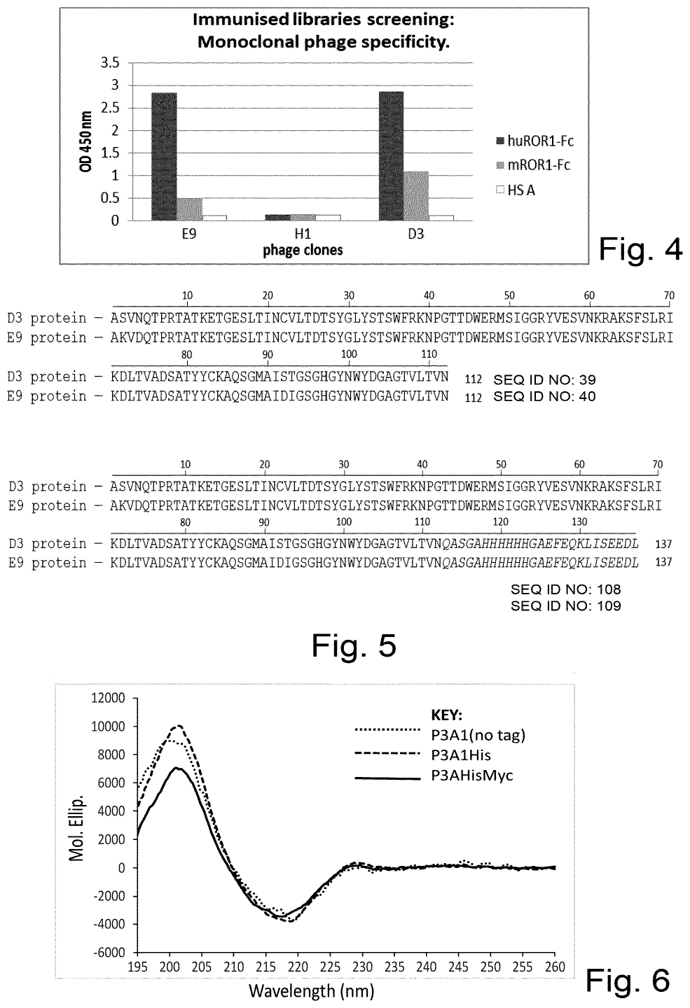

Recombinant mouse ROR1 protein was used for screening the immunised libraries (ELSI 5-7). Following a protocol similar to that used to screen the synthetic library, VNAR domains were isolated after 3 rounds of selection using biotinylated ROR1 antigen immobilised on streptavidin-coated beads. Following the selection process, 45% of monoclonal phage displaying a VNAR domain (from the combined output from the 3 libraries) was specific to human and mouse ROR1. One third of the ROR1 specific VNAR were found to have the sequence D3 (FIGS. 4 and 5) and the remaining two thirds—to the sequence E9 (FIGS. 4 and 5).

The sequences obtained from screening with mouse ROR1 are E9 and D3. (FIG. 5)

All lead anti-ROR1 VNAR proteins were expressed in TG1 E. coli or HEK293 mammalian cells and IMAC purified from the periplasmic fraction or the cell supernatant, respectively.

### Methods

### Ignar Titre in Sera ELISA

ELISA were carried out using the following protocol:
1. Coat an ELISA plate with 100 μl/well of 1 mg/ml of human ROR1-Fc or mouse ROR1-Fc in or PBS. Incubate at 4° C. overnight.
2. Wash plates 3× with PBST.
3. Block plates by adding 200 μl/well 2% (w/v) M-PBS and incubate at 37° C. for 1 h.
4. Wash plates 3× with PBST.
5. Serially dilute dogfish sera in PBS from no less than 1:10 up to 1:1000 and add 100 μl/well. Incubate at room temperature for 1 h.
6. Wash plates 3× with PBST.
7. Add 100 μl/well primary antibody (mouse monoclonal anti-IgNAR antibody, GA8) diluted as hybridoma tissue culture supernatant in PBST.
8. Wash plates 3× with PBST.
9. Add 100 μl/well of a suitable secondary anti-mouse IgG HRP conjugate diluted in PBS. Incubate for 1 h.
10. Wash plates 2× with PBST followed by 2× with PBS.
11. Add 100 μl/well of TMB substrate to the plate and incubate until the appearance of signal/onset of saturation. Stop the colour development by adding 100 μl/well of 0.18 M H2SO4.
12. Read at 450 nm with a microtiter plate reader.

### Library Screening

1. To rescue library phage for selections, cultures from library glycerol stocks were grown at 37° C. and 250 rpm, in 2×TY, 2% glucose, 100 μg/ml ampicillin to an OD600 of 0.5.

2. Cells were super-infected with 109 M13K07 helper phage (NEB) and then incubated overnight in 2×TY, 100 μg/ml ampicillin, 50 μg/ml kanamycin at 25° C. and 250 rpm.

3. The phage was PEG-precipitated (20% PEG/2.5 M NaCl) twice from the bacterial culture and the resulting phage pellets were resuspended in 1 ml PBS.

4. 200 μl of Dynabeads M-280 Streptavidin (Invitrogen #11205D), pre-blocked with 2% (w/v) MPBS, were coated with 400 nM biotinylated mouse ROR1 rotating at 20 rpm, at room temperature for 1 h.

5. Library phage was de-selected by incubation with Dynabeads for 1 h rotating at room temperature and then added to the antigen-coated beads.

6. Beads were washed 5-10 times with PBST and 5-10 times with PBS, eluted by rotating for 8 min in 400 μl 100 mM TEA and neutralised by the addition of 200 μl 1 M Tris-HCl pH 7.5.

7. E. coli TG1 cells (10 ml) were infected with 300 μl of eluted phage for 30 min at 37° C. and grown overnight at 37° C. on TYE agar plates containing 2% (w/v) glucose and 100 μg/ml ampicillin.

8. Three further rounds of selection were conducted and outputs were screened for antigen-specific binding by monoclonal phage and periplasmic extract ELISAs against human or mouse ROR1. Phage binders were detected using HRP-conjugated anti-M13 antibody (GE Healthcare, 27942101) and periplasmic protein was detected using HRP-conjugated anti-c-Myc antibody (Roche, 118 141 50 001).

### VNAR Expression in E. Coli

1. Dilute the overnight culture 1:50 in TB media with phosphate salts, 1% glucose, 100 μg/ml Ampicillin and incubate at 37° C. with vigorous shaking (250 rpm) all day.

2. Pellet the cells by centrifugation at 3,000×g for 20 min at 20° C.

3. Re-suspend the cells in the same volume of TB media with phosphate salts, 100 μg/ml Ampicillin (no glucose).

4. Add IPTG to a final concentration of 1 mM IPTG and incubate at 16° C. overnight (16 h) with shaking at 250 rpm.

5. Collect the cells by centrifugation at 6,000×g for 30 min (the pellet could be frozen at this point at −20° C.).

6. Re-suspend the pellet in 10% culture volume ice-cold TES and shake gently on ice for 15 min.

7. Add an equal volume ice-cold 5 mM MgSO4 (for 2.5 mM final concentration of MgSO4) and continue shaking gently on ice for a further 15 min.

8. Pellet the suspension by centrifugation at 15,000×g for 30 min at 4° C. and carefully decant the supernatant containing released periplasmic proteins into a clean falcon.

9. Add 10×PBS pH 7.4 [final concentration of 1×PBS] to peri-prep extract prior to IMAC incubation.

### VNAR Expression in HEK293

10 μg DNA in water (sterile filtrated) for 10 ml culture.

Use 10 ml of cells (˜106/ml) in a 50 ml bioreactor tube (exponentially growing cells in fresh media)

Add OptiMEM media to DNA to a total volume of 500 μl.

Add 25 μl of PEI (1 mg/ml stock made up in water) to a separate 500 μl OptiMEM media.

Incubated DNA and PEI at room temperature for up to 15 min.

Mix 500 μl of PEI in media to each 500 μl of DNA in media.

Incubated at room temperature for 20-30 min facilitating complex formation.

Add 1 ml of mixture to the cells and incubate at 37° C., 5% CO2 sharking 140 rpm.

Next day feed cells by addition of 250 μl of 20% (w/v) tryptone to 10 ml of cells to obtain the final concentration of tryptone 0.5%

Leave cells to express for 3-5 days.

Spin the cells and assess supernatant for secreted protein to determine productivity.

Add 10×PBS pH 7.4 [final concentration of 1×PBS] to peri-prep extract prior to IMAC incubation.

This protocol can be scaled up or down as required for protein production.

### Protein Expression (Scale Up)

ROR1 binding VNAR proteins expressed well in many different forms in several different expression systems. The addition of standard C terminal tags, including His and His6Myc, to aid protein purification, handling and protein analysis, did not affect the binding of ROR1 VNARs to target ROR1 (Table 2).

TABLE 2
SPR data for binding of VNARs with different
C-terminal tags to human ROR1 and ROR2

| | | hROR1 | | | |
|---|---|---|---|---|---|
| VNAR | C-terminal tag | Ka (M⁻¹s⁻¹) | Kd (s⁻¹) | K_{D} (nM) | hROR2 |
| B1 | 6xHis | 2.33E+06 | 1.91E−04 | 0.11 | No binding |
| | 6xHis myc | 7.47E+05 | 6.09E−04 | 0.83 | No binding |
| P3A1 | 6xHis | 2.92E+06 | 2.06E−02 | 7.8 | No binding |
| | 6xHis myc | 9.8E+05 | 2.5E−02 | 25.6 | No binding |
| P3A1 | No tag | 1.67E+06 | 5.98E−04 | 0.36 | No binding |
| dimer | 6xHis myc | 2.08E+06 | 6.37E−04 | 0.35 | No binding |

In addition, VNAR C-terminal tags do not affect VNAR structure as measured by circular dichroism (FIG. 6—CD spectra of VNARs) (Glasgow University, UK).

VNARs were also expressed genetically fused to mouse and human IgG Fc sequences, and as N-terminal fusions to engineered inteins, enabling site specific conjugation to labels and drugs. Expression systems used include E. coli (periplasmic and cytoplasmic expression), HEK 293 and CHO (Evitria Fc fusion proteins).

### Example 2—VNAR Reformatting

### Homodimers

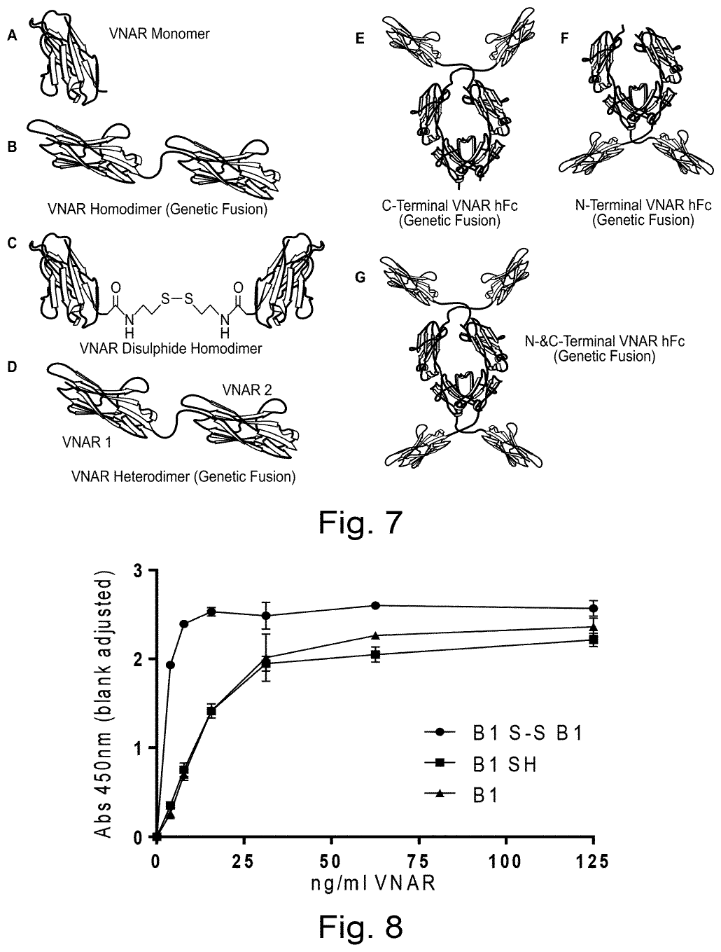

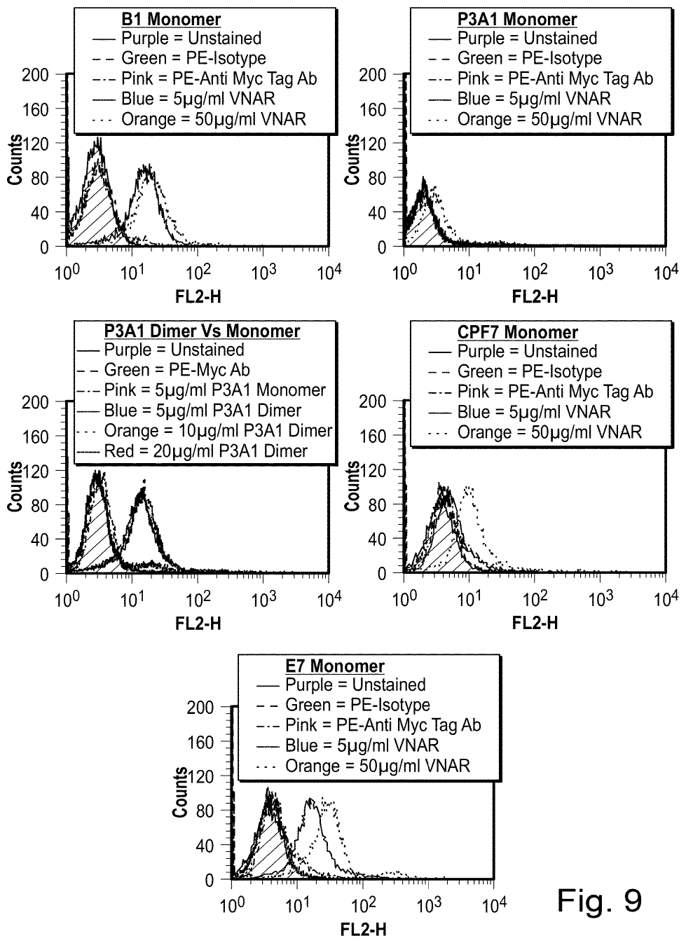

ROR1 binding VNARs were successfully reformatted into homodimers by genetic fusion using standard GlySer based linkers (FIG. 7B). Homodimers were shown to have increased affinity for recombinant hROR1 by SPR and ELISA, and increased binding to cell surface ROR1 on ROR1 positive cancer cell lines by flow cytometry (FIG. 9). Flow cytometry experiments are described in more detail in Example 4.

In addition, ROR1 binding VNAR homodimers were successfully generated through chemical conjugation. VNARs were expressed as intein fusion proteins and cleaved with cysteamine to generate C-terminal thiol derivatives, which then self-associated into homodimers via C terminal intermolecular disulphide formation (FIG. 7C). These disulphide linked homodimers showed increased binding affinity to recombinant hROR1 by ELISA (FIG. 8). Production of intein fusion proteins is discussed in more detail in Example 8.

### Heterodimers

ROR1 binding VNAR heterodimers were generated by genetic fusion with standard GlySer linkers or alternatively with PGVQPSPGGGGS (SEQ ID NO: 63) or PGVQPAPGGGGS (SEQ ID NO: 64) linkers (FIG. 7D) and demonstrated high affinity specific binding to recombinant ROR1 and ROR1 positive cells. Heterodimeric VNAR proteins can also be generated by chemical conjugation.

Results for binding characterisation experiments are tabulated in Table 3 and 4 (see Example 3) and Tables 18, 19, 20 (see Example 10).

### VNAR Fc Fusion Proteins

Fusion of proteins to an Fc domain can improve protein solubility and stability, markedly increase plasma half-life and improve overall therapeutic effectiveness. ROR1 binding VNARs were genetically fused to the N terminus of mouse IgG2a Fc (mFc) and both the N and C termini of human IgG1 (hFc) via standard GlySer linkers (FIG. 7 E, F, G). Examples of Fc sequences

Mouse IgG2a Fc (mFc)
(SEQ ID NO: 95)
EPRGPTIKPCPPCKCPAPNLLGGPSVFIFPPKIKDVLMISLSPIVTCVVVDVSEDDPDVQISWFVNNVEVHT
AQTQTHREDYNSTLRVVSALPIQHQDWMSGKEFKCKVNNKDLPAPIERTISKPKGSVRAPQVYVLPPPEE
EMTKKQVTLTCMVTDFMPEDIYVEWTNNGKTELNYKNTEPVLDSDGSYFMYSKLRVEKKNWVERNSYSC
SVVHEGLHNHHTTKSFSRTPGK
Human IgG1 Fc (hFc)
(SEQ ID NO: 96)
EPKSSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFS
CSVMHEALHNHYTQKSLSLSPGK

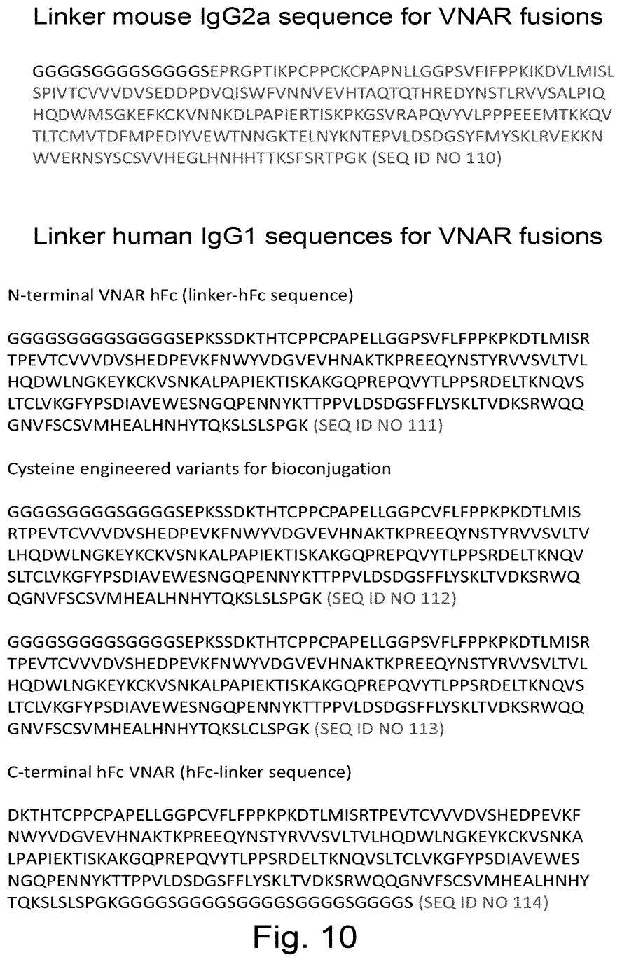

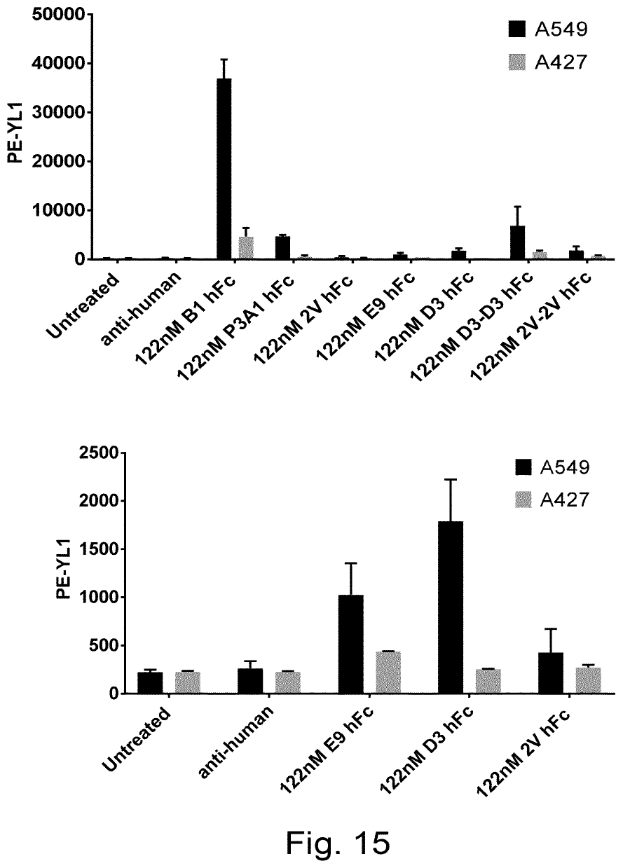

VNAR Fc fusion proteins were expressed as secreted protein in CHO K1 cells and purified from the media using MabSelect™ SuRe™ (Evitria, Switzerland). Purified proteins were analysed by SEC (AdvanceBio, Agilent), SDS PAGE and mass spectrometry to confirm sequence and protein integrity. The resulting VNAR Fc fusion proteins bind recombinant human ROR1 by SPR (Table 6) and ROR1 positive cells with high affinity (FIG. 15) and were shown to internalise into ROR1 positive cells. ROR1 binding VNARs were also genetically fused to engineered hIgG1 Fc fusion proteins that incorporated an engineered cysteine substitution in the hIgG1 Fc sequence, for example at position S252C or S473C (Kabat numbering) to enable site specific labelling (FIG. 10).

### Typical Method for Expression of VNAR Intein Fusion Proteins

For expression as intein fusions, DNA encoding VNARs was optimised for E. coli expression (GeneArt, Thermo) and cloned into the Ndel/Sapl sites of the pTXB1 vector (NEB) and derivatives thereof. This results in a gene encoding the VNAR protein of interest fused to an engineered intein domain which in turn is fused to a chitin binding domain (CBD) to enable purification on a chitin column. pTXB1 vector derivatives encode alternative inteins as the fusion proteins.

Transformed E. coli cells were grown in 1 L shaker flasks until OD600=˜0.6, cold shocked 4° C. for 2 hours then protein expression induced with 0.5 mM IPTG at 18° C. overnight. Cells were lysed by sonication in lysis buffer (50 mM sodium phosphate pH7.4, 0.5 M NaCl, 15% glycerol, 0.5 mM EDTA, 0.1% Sarkosyl, 1 mM AEBSF) and centrifuged to remove cell debris. VNAR intein fusion protein was purified from clarified cell lysate by immobilising on chitin beads (NEB, S6651). Beads were washed extensively with lysis buffer followed by cleavage buffer (50 mM sodium phosphate pH6.9, 200 mM NaCl) and VNARs released from the beads by overnight chemical cleavage in 400 mM dioxyamine, or O,O′-1,3-propanediylbishydroxylamine, or 100 mM cysteine or cysteamine to generate the corresponding C-terminal aminoxy, C-terminal cysteine or C-terminal thiol derivative of the VNARs (FIG. 11).

Cleaved VNAR supernatant was then further purified by SEC (Superdex75 26/60 GE healthcare) and/or IMAC (HisTrap HP, GE Healthcare). Concentrations were determined from absorbance at 280 nm using the theoretical extinction coefficient predicted from the amino acid sequence. All proteins were characterised by reducing and non-reducing SDS PAGE analysis and mass spectrometry. The formation of the desired disulphide bond was confirmed by mass spectrometry methods.

### Example 3—Anti-ROR1 VNAR Characterisation—

### Binding to ROR1 and ROR2 by SPR and ELISA

### Species Cross-Reactivity of ROR1 VNAR Binders

Soluble VNAR protein clones (B1, P3A1 and D3) were analysed for species cross-reactivity with human, mouse and rat ROR1 along with a positive control antibody 2A2 and an anti ROR2 specific antibody control. 2A2 is an anti-human ROR1 specific mouse monoclonal antibody (BioLegend Cat #2357802) and the anti ROR2 antibody is a commercial monoclonal mouse antibody from R&D Systems (Cat #MAB2064).

VNAR B1 was observed to be a very strong binder to both mouse and human ROR1. All VNARs are species cross-reactive to ROR1 derived from a human, mouse and rat origin (Table 3 and Table 4). None of the VNAR clones cross-reacted with human ROR2 (Table 3).

### Determination of Binding Kinetics to Human ROR1, Human ROR2, Mouse ROR1 or Rat ROR1

Binding kinetics were determined using a Pioneer Surface Plasmon Resonance (SPR) instrument (SensiQ/Pall ForteBio). ROR1-hFc or ROR2-hFc fusion proteins (extracelluar domains) were immobilised in sodium acetate pH5 buffer to COOH₂ chips using amine coupling. VNARs and VNAR-Fc molecules were tested at various concentrations and the Ka (M⁻¹s⁻¹), Kd (s⁻¹) and KD (nM) values were determined using QDat software (SensiQ/Pall ForteBio). ROR1 2A2 mAb (Biolegend) and ROR2 mAb (R&D Systems) were included as controls for positive/negative binding to ROR1 and ROR2. 2V is a control VNAR sequence, derived from a naïve VNAR library, so is representative of this protein class but has no known target.

TABLE 3
SPR data for binding of VNAR molecules to human ROR1 (hROR1) and human ROR2
(hROR2). C-terminal His₆ or His₆Myc tagged VNARs were expressed.

| | Expression | hROR1 | | | |
|---|---|---|---|---|---|
| VNAR | System | Ka (M⁻¹s⁻¹) | Kd (s⁻¹) | KD (nM) | hROR2 |
| B1 | E. coli | 6.29E+05 | 7.93E−04 | 1.6 | No binding |
| B1 | HEK293 | 5.36E+05 | 2.26E−03 | 0.63 | No binding |
| P3A1 | E. coli | 2.47E+06 | 4.42E−02 | 19.1 | No binding |
| CPF7 | E. coli | 2.33E+06 | 2.96E−02 | 13.6 | No binding |
| E7 | E. coli | 1.11E+06 | 1.18E−02 | 11.1 | No binding |
| D3 | E. coli | 2.09E−05 | 3.24E−02 | 159.1 | No binding |
| D3 | HEK293 | 1.39E+06 | 7.52E−02 | 54.5 | No binding |
| E9 | HEK293 | 4.23E+05 | 4.45E−02 | 136.6 | No binding |
| P3A1-[G₄S]₅-P3A1 | HEK293 | 4.9E+06 | 1.12E−03 | 0.30 | No binding |
| D3-[G₄S]₅-D3 | E. coli | 2.95E+06 | 3.38E−03 | 2.33 | No binding |
| P3A1-[G₄S]₅-B1 | E. coli | 3.13E+06 | 2.08E−03 | 1.0 | No binding |
| P3A1-[G₄S]₃-B1 | E. coli | 1.09E+06 | 2.84E−03 | 2.7 | No binding |
| P3A1-[G₄S]₇-B1 | E. coli | 1.49E+06 | 6.44E−03 | 4.3 | No binding |
| 2V | E. coli | No binding | No binding | No binding | No binding |
| 2V-[G₄S]₅-2V | E. coli | No binding | No binding | No binding | No binding |

TABLE 4
SPR data for binding to mouse ROR1 (mROR1) and rat ROR1 (rROR1)

| | | mROR1 | | | rROR1 | | |
|---|---|---|---|---|---|---|---|
| | Expression | Ka | Kd | KD | Ka | Kd | KD |
| VNAR | System | (M⁻¹s⁻¹) | (s⁻¹) | (nM) | (s⁻¹) | (M⁻¹s⁻¹) | (nM) |
| B1 | E. coli | 4.32E+05 | 2.09E−03 | 5.2 | 1.2E+05 | 1.11E−02 | 94.5 |
| B1 | HEK293 | 7.2E+05 | 1.51E−03 | 2.18 | 1.16E+05 | 6.51E−03 | 56.5 |
| P3A1 | E. coli | 2.95E+06 | 4.08E−02 | 14.3 | 2.86E+06 | 4.5E−02 | 17.7 |
| CPF7 | E. coli | 2.26E+06 | 3.2E−02 | 19.1 | 7.72E+05 | 3.66E−02 | 68.6 |
| E7 | E. coli | 1.41E+06 | 2.0E−03 | 1.4 | ND | ND | ND |
| P3A1- | HEK293 | 4.17E+06 | 1.45E−03 | 0.396 | 3.18E+06 | 1.73E−03 | 0.57 |
| [G₄S]₅- | | | | | | | |
| P3A1 | | | | | | | |
| 2V | E. coli | No | No | No | No | No | No |
| | | binding | binding | binding | binding | binding | binding |
| 2V- | E. coli | No | No | No | No | No | No |
| [G₄S]₅-2V | | binding | binding | binding | binding | binding | binding |

VNAR proteins have been developed, which bind with high affinity to human ROR1 ECD in monomeric and multimeric formats (both homo and hetero dimeric forms), show no binding to the closely related family member human ROR2 and cross react with high affinity to mouse and rat orthologues of ROR1. Reformatting the P3A1 and D3 proteins as dimers significantly increased the binding affinity to human ROR1 with a significant reduction in the dissociation rate constants being observed.

The binding of a chemically conjugated B1 homodimer to hROR1 was also assessed by ELISA. To generate this molecule a B1 derivative was generated with a unique C-terminal thiol functionality through chemical cleavage of the corresponding B1-intein fusion protein precursor with cysteamine. Intermolecular disulphide bond formation was used to covalently link the C-termini of the two proteins to generate a homodimer of unnatural but defined topology (B1-S—S—B1, FIG. 7C). Binding of the B1-S—S—B1 to hROR1 was compared to the B1 monomer by ELISA.

In brief, ELISA method as follows. Wells coated with 100 ng antigen and incubated, covered, at room temperature for 2 hr. Plates washed 3×400 μl per well with PBST (PBS+0.05% Tween 20 (v/v)), then blocked with 4% skimmed milk powder (w/v) in PBST for 1 hour at 37° C. Plates washed as before plus additional wash in PBS alone. Binding proteins were diluted in 4% milk PBST and incubated overnight at 4° C. Plates washed 3× with PBST, 3×PBS and binding detected using appropriate secondary detection antibody in 4% milk PBST, room temperature 1 hour. Secondary antibodies used include:
Anti-c-Myc, HRP (Invitrogen #R951-25)
Rabbit anti-Human IgG H&L, HRP (Abcam #ab6759)
Rabbit anti-Mouse IgG H&L, HRP (Abcam #ab97046)
Mouse anti-polyHis, HRP (Sigma #A7058)
   Plates washed 3× with PBST. 100 μL TMB substrate (Thermo #34029) added and reaction allowed to proceed at r.t. for 10 mins. 100 μL of 2M H₂SO₄ added to quench the reaction. Plate centrifuged briefly before absorbance at 450 nm read on a CLARIOstar plate reader (BMG Labtech).
   Whilst B1 monomer and the C-terminal thiol derivative binds strongly to human ROR1, an increase in human ROR1 binding was observed for the chemically linked B1-S—S—B1 dimer (FIG. 8).

### Example 4—Anti-ROR1 VNAR Characterisation—Cell Binding and Internalisation by Flow Cytometry

### Cell Surface Binding

Adherent human cancer cells were detached from tissue culture flasks by incubating with 0.1% EDTA/PBS solution at 37° C. for ˜10 minutes or until cells detached easily. Cells were re-suspended in 5 ml ice-cold PBS/2% FCS in 15 ml tubes and centrifuged at 1500 rpm for 5 mins at 4° C. Supernatant was removed and the cell pellet re-suspended in 1-2 ml of PBS/2% FCS. A cell count was performed using a Z1 Coulter Particle Counter (Beckman Coulter) and 5×10\hat{ }5 cells were aliquoted per test sample. Cells were incubated with 100 μl of either VNAR (His6Myc tagged), VNAR-Fc molecules or ROR1 mAb and IgG controls for 1 hour on ice. Excess VNAR, VNAR-Fc or mAb was removed by adding 5 ml of ice-cold PBS/2% FCS, followed by centrifugation at 1500 rpm for 5 mins at 4° C. The supernatant was removed and a second wash performed by re-suspending the cell pellet in 1 ml of ice-cold PBS/2% FCS and adding a further 4 ml of ice-cold PBS/2% FCS. Samples were again centrifuged at 1500 rpm for 5 min at 4° C. Supernatant was removed and excess liquid removed by blotting the tubes on tissue paper. Appropriate secondary antibodies were used to detect bound VNAR (His₆Myc), VNAR-hFc, VNAR-mFc or ROR1 mAb (PE-anti-Myc tag antibody (CST), PE-anti-human antibody (JIR labs/Stratech), and PE-anti-mouse antibody (JIR/Stratech) respectively). Cells were incubated with chosen secondary antibody for 30 min on ice. Cells were washed to remove excess antibody as described earlier. Cell pellets were re-suspended in 0.5 ml of ice-cold PBS/2% FCS and left on ice in the dark prior to analysis on either a FACS Calibur (BD Biosciences) or an Attune NxT (ThermoFisher) flow cytometer.

### Binding of VNARs to a Panel of Cancer Cell-Lines

FIG. 9 shows representative flow cytometry histograms for binding of anti ROR1 VNARs binding to the ROR1^{hi} A549 lung adenocarcinoma cells.

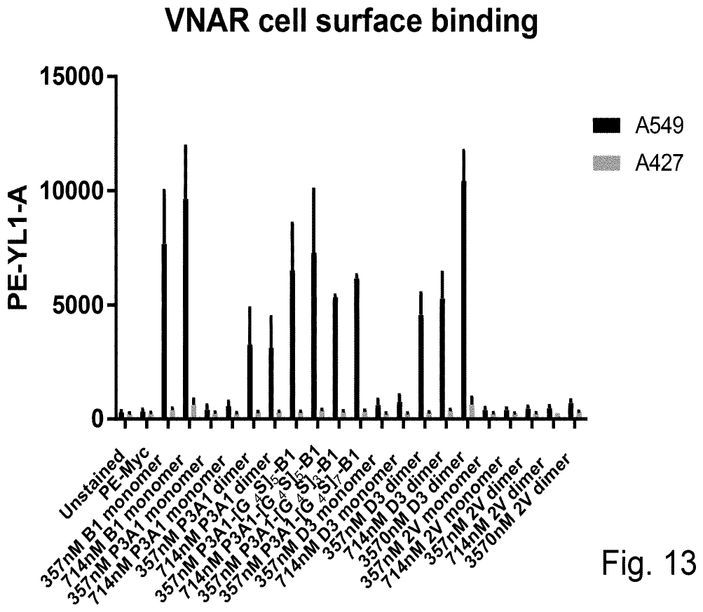

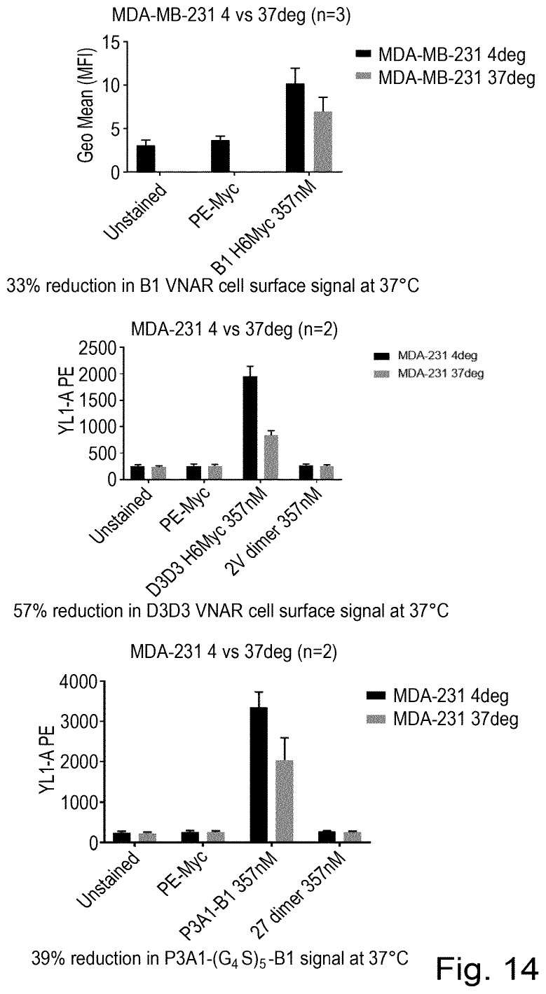

FIG. 13 shows the binding of different VNARs to the ROR1^{hi} A549 lung adenocarcinoma cells and the ROR1^{low} lung cancer cell-line A427 by flow cytometry at a fixed concentration of protein.

TABLE 5
Relative ranking of VNAR cell surface binding in human
cancer cell lines, ascertained by flow Cytometry.

| | Based on Median (YL1-PE) or Geo Mean (FL2-PE) | | | | | |
|---|---|---|---|---|---|---|
| | | | MDA- | | | |
| Mole- | A549 | A427 | MB-231 | T47D | HT-29 | Colo205 |
| cule | (ROR1^{hi}) | (ROR1^{low}) | (ROR1^{hi}) | (ROR1^{low}) | (ROR1^{hi}) | (ROR1^{low}) |
| B1 | +++++ | + | +++ | ++ | +++ | + |
| E7 | ++++ | + | +++ | ++ | +++ | + |
| P3A1 | + | − | + | +/− | + | − |
| CPF7 | ++ | − | + | +/− | + | − |
| P3A1- | +++ | − | ++ | / | / | / |
| [G₄S]₅- | | | | | | |
| P3A1 | | | | | | |
| dimer | | | | | | |
| CPF7- | +++ | ++ | / | / | / | / |
| [G₄S]₅- | | | | | | |
| CPF7 | | | | | | |
| dimer | | | | | | |
| P3A1- | ++++ | − | +++ | / | / | / |
| [G₄S]₅- | | | | | | |
| B1 | | | | | | |
| D3 | + | − | / | / | / | / |
| D3- | ++++ | − | ++ | / | / | / |
| [G₄S]₅- | | | | | | |
| D3 | | | | | | |
| dimer | | | | | | |
| 2V | − | − | − | − | − | − |
| 2V- | − | − | − | − | − | − |
| [G₄S]₅- | | | | | | |
| 2V | | | | | | |
| dimer | | | | | | |

Number of
‘+’ corresponds to binding strength.
‘−’ indicates no binding.
‘/’ not determined in this cell line.

Robust binding of the VNARs to ROR1 expressing cancer cell-lines is observed as compared to the ROR1^{low} cancer cell-lines where little to no staining was observed for the majority of the ROR1 binding VNARs tested.

The cell-surface staining for P3A1-P3A1 is not as strong as for B1 or D3-D3 proteins, which may reflect differences in the epitopes of these binders and that in the cellular context some regions of the extracellular domain of ROR1 are potentially more accessible for binding than others.

### Cell-Surface Staining Following Incubation at 37° C. vs 4° C.

Briefly, 5×10\hat{ }5 MDA-MB-231 cells were incubated with VNAR, VNAR-Fc, ROR1 2A2 mAb or IgG1 control for 1 hr on ice. Cells were washed twice by addition of 5 ml of ice-cold PBS/2% FCS followed by centrifugation at 1500 rpm for 5 mins at 4° C. Following the final centrifugation step, excess supernatant was removed and the tubes blotted on tissue paper. Each cell pellet was re-suspended in 200 μl of PBS/2% FCS and either placed on ice or at 37° C. for 2 hours. Bound VNAR (His₆Myc tagged), VNAR-hFc, VNAR-mFc or ROR1 2A2 mAb was detected using either PE-conjugated anti-Myc tag antibody (CST), PE-conjugated anti-human antibody (JIR/Stratech) or PE-conjugated anti-mouse antibody (JIR/Stratech). Loss of signal at 37° C. with respect to samples incubated on ice is indicative of ROR1 internalisation.

A decrease in cell-surface binding after incubation at 37° C. versus 4° C. was observed for antiROR1 VNAR constructs both as monomer and as multimers, comprising the same or different VNAR binding modules, (FIG. 14), which is consistent with binding and internalisation of the proteins by ROR1.

### Example 5—Characterisation of Anti-ROR1 VNAR-Fc Fusion Proteins—

### Binding to ROR1 and ROR2 by SPR and Cell Surface Binding and Internalisation

ROR1 binding VNARs were expressed fused to the N terminus of mouse IgG2a Fc (mFc) and the N terminus and C-terminus of human IgG1 (hFc) via standard GlySer linkers. Fusion of the human IgG1 Fc were also generated whereby Ser252 in the Fc region (Kabat numbering) was replaced with a Cys (FIG. 10).

### Binding to ROR1 and ROR2 by SPR

Using the procedures outlined above the binding of VNAR-Fc fusions to human, mouse and rat ROR1 and human ROR2 were determined by SPR.

TABLE 6
SPR data for binding of VNAR-Fc
fusions to human ROR1 and human ROR2

| | hROR1 | | | |
|---|---|---|---|---|
| Molecule | Ka (M⁻¹s⁻¹) | Kd (s⁻¹) | KD (nM) | hROR2 |
| B1 mFc | 4.19E+05 | 3.356E−04 | 0.8 | No binding |
| 2V mFc | No binding | No binding | No binding | No binding |
| B1 hFc | 3.08E+06 | 9.53E−05 | 0.032 | No binding |
| P3A1 hFc | 1.07E+07 | 5.64E−04 | 0.084 | No binding |
| D3 hFc | 1.21E+06 | 2.88E−03 | 2.6 | No binding |
| E9 hFc | 7.07E+05 | 3.64E−03 | 5.3 | No binding |
| D3-D3 hFc | 4.96E+06 | 9.88E−04 | 0.25 | No binding |
| hFc-P3A1 | 2.38E+06 | 7.76E−04 | 0.35 | No binding |
| hFc-D3 | 1.10E+06 | 2.35E−03 | 2.37 | No binding |
| hFc-D3-D3 | 2.35E+06 | 1.01E−03 | 0.49 | No binding |
| 2V hFc | No binding | No binding | No binding | No binding |
| 2V-2V hFc | No binding | No binding | No binding | No binding |

As shown in Table 6 anti ROR1 VNAR-Fc proteins bind with high affinity to human ROR1, with no binding to human ROR2 observed. Strong binding to mouse and rat ROR1 ECD was also observed.

As VNAR-Fc fusions, a significant decrease in the K_{D} apparent values for ROR1 binding is observed with respect to the corresponding VNAR monomers. This is consistent with these VNAR-Fc fusions binding in a bivalent fashion to the ROR1-chip surface in the SPR experiments. Both N- and C-terminal VNAR Fc fusions bind with high affinity to human ROR1 but do not bind to human ROR2.

### Binding of VNARs to Cancer Cell-Lines

Binding of the VNAR-Fc fusions to the surface of a panel of cancer cell lines was measured by flow cytometry using the methods outlined previously. FIG. 15 shows the binding of different VNAR-Fc fusions to the ROR1^{hi} A549 lung adenocarcinoma cells and the ROR1^{low} lung cancer cell-line A427 by flow cytometry at a fixed concentration of protein.

Table 7 summarises the binding data for VNAR-Fc proteins with a variety of ROR1^{hi} cancer cell-lines.

TABLE 7
Relative ranking of VNAR hFc molecule cell surface binding in
ROR1^{hi} human cancer cell lines. The number of ‘+’ indicates the
strength of binding. ‘−’ indicates no binding. ‘/’ indicates that it
has not been determined. hFc molecules were detected using a
PE-anti-human antibody (Jackson Immune Research/Stratech)
and a ThermoFisher Attune NxT flow cytometer.

| | Based on Median (YL1-PE) | | | |
|---|---|---|---|---|
| Molecule | A549 | MDA-MB-231 | PC-9 | NCI-H1975 |
| B1 hFc | ++++ | +++++ | +++++ | +++++ |
| P3A1 hFc | ++ | +++ | ++ | ++ |
| D3 hFc | + | ++ | / | / |
| E9 hFc | + | / | / | / |
| D3-D3 hFc | ++ | ++++ | / | / |
| 2V hFc | − | − | − | − |
| 2V-2V hFc | + | / | / | / |

Robust binding of the VNARs to ROR1 expressing cancer cell-lines is observed as compared to the ROR1^{low} cancer cell-lines, where little to no staining was detected for the majority of the ROR1 binding VNARs tested.

Differences in the mean cell-surface staining may indicate that different regions of ROR1 may be more accessible than others when the protein is expressed on the cell surface. For targeting less accessible regions of ROR1 on cancer cells, it would be advantageous to use small protein binders such as VNARs as opposed to large antibodies that will be sterically occluded.

### Cell-Surface Staining Following Incubation at 37° C. vs 4° C.

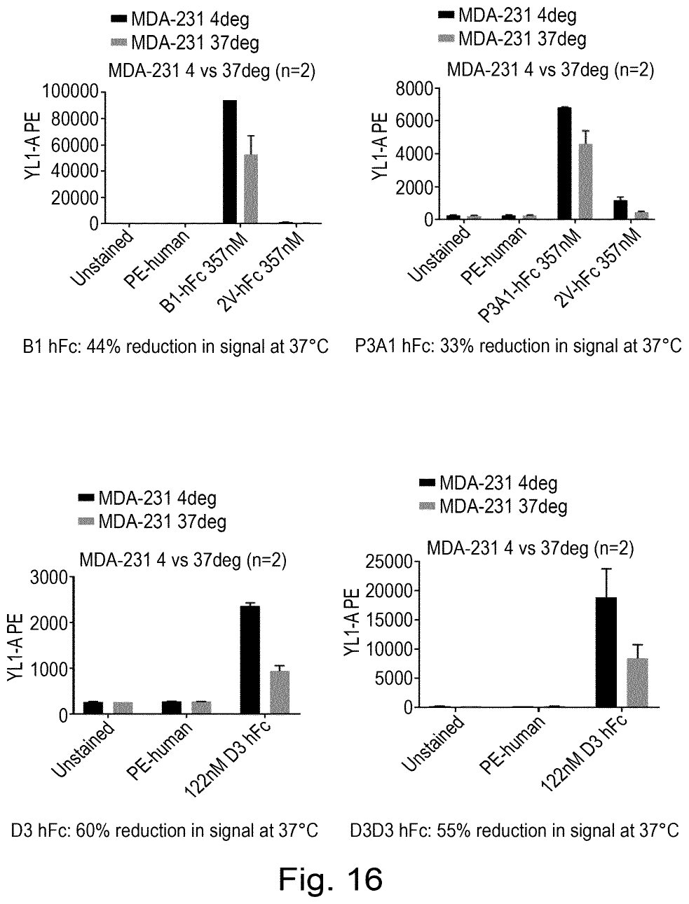

The binding of VNAR-Fc fusions to MDA-MB-231 cells after incubation at 37° C. or 4° C. was determined by flow cytometry using the methods described previously. For the B1-hFc, P3A1-hFc, D3-hFc and D3D3-hFc proteins tested there was a loss of cell-surface staining after incubation at 37° C. versus 4° C. (FIG. 16), consistent with binding and internalisation of these VNAR-hFc fusion proteins.

### Internalisation by Immunofluorescence Following Incubation at 37° C. VS 4° C.

The cellular localisation of human IgG1 Fc and mouse IgG2a Fc fusion proteins can be detected by immunofluorescence using fluorescently labelled secondary antibodies targeting these domains. Immunofluorescence methods were used to detect internalisation of VNAR-Fc by ROR1 on cancer cells.

Black, clear bottom 96-well plates (Greiner) were coated with 100 μg/ml Collagen I (Sigma) to aid cell attachment. Cells were seeded in complete growth media (Gibco) into the coated 96 well plates and incubated at 5% CO2, 37° C. for 24 hr. The media was removed and replaced with serum-free media (Gibco) on the following day and left overnight. On the following morning, media was removed and cells were treated with various concentrations of VNAR-Fc, ROR1 2A2 mAb or IgG1 negative control (both BioLegend). Plates were incubated on ice for 1 hour. Treatments were removed and replaced with 100 μl of PBS/2% FCS per well. One plate was kept on ice and the other was placed at 37° C., 5% CO2 for 2 hours. Following this 2 hour incubation, the PBS/2% FCS solution was removed and cells were fixed with 4% Paraformaldehyde in ice cold PBS for 20 min on ice. The PFA solution was removed and replaced with 0.05% Saponin (Sigma) made up in PBS/2% FCS for 15 min at room temperature. This step permeabilises the cell membranes. Secondary antibody staining was performed using; AF488-anti-human Ab (1:250; ThermoFisher) to detect VNAR-hFc fusion proteins and AF488-anti-mouse Ab (1:500; CST) to detect the VNAR-mFc fusion molecule and ROR1 2A2 mAb. All secondary antibody working stocks were made up in 0.05% Saponin/PBS/2% FCS. Plates were incubated at 4° C. overnight in the dark. On the following day, secondary AF488-conjugated antibodies were removed and the cells were washed ×3 using 0.05% Saponin/PBS/2% FCS. Lamp-1 antibody (1:200; CST) or EEA1 antibody (1:50; Santa Cruz) were added to detect lysosome and early endosome compartments respectively. Plates were incubated in the dark at room temperature for 2 hours. The Lamp-1 and EEA1 antibodies were then removed and the cells were washed X3 with 0.05% Saponin/PBS/2% FCS. AF647-anti rabbit antibody (1:1000; CST) was then added to detect Lamp1 and EEA1 antibody binding. A further incubation in the dark at room temperature for 2 hours was performed before removing the AF647-secondary antibody and washing the cells X3 with 0.05% Saponin/PBS/2% FCS. Cell nuclei were stained using 10 μM Hoechst reagent (Sigma) in 0.05% Saponin/PBS/2% FCS for 20 min at room temperature in the dark. Finally, this solution was removed and replaced with PBS. Plates were stored at 4° C. in the dark prior to imaging using a GE Healthcare InCell 2000 instrument.

Internalisation of B1 hFc and B1 mFc was observed in MDA-MB-231 breast cancer cells following incubation at 37° C. for 2 hours. The VNAR-Fc-ROR1 complex appears to overlay with Lamp-1 and EEA1 staining following internalisation which is suggestive of ROR1 cellular trafficking via early endosomal and lysosomal compartments. ROR1-VNAR-Fc staining remained predominantly at the cell surface when the samples were incubated on ice for 2 hours. No cell surface binding or internalisation was observed following incubation with 2V Fc protein (non-binding negative control VNAR). B1-hFc and B1-mFc were not internalised by the ROR1^{low} lung cancer cell-line A427.

### Example 6—Humanisation and Further Engineering

A number of humanised sequence derivatives of two lead ROR1 binding VNARs were generated using two different strategies.

Humanised sequences were designed based on the human germ line VK1 sequence, DPK-9. For example, in P3A1 V1 the framework regions 1, 3 and 4 of the VNAR were mutated to align with the framework regions of DPK-9.

The second strategy involved grafting the binding loops of the ROR1 binding VNARs onto a previously humanised VNAR framework (Kovalenko et al JBC 2013 288(24) 17408-17419; WO2013/167883). For the first construct (G₁) only the CDR1 and CDR3 loops were grafted. The second construct (G2) had both the CDRs and HV loops grafted.

Humanised D3 sequences were designed using a combination of approaches.

Examples of humanised/grafted VNAR sequences:

B1 G1
(SEQ ID NO: 45)
TRVDQSPSSLSASVGDRVTITCVLTGANYGLASTYWYRKNPGSSNKEQI
SISGRYSESVNKGTKSFTLTISSLQPEDSATYYCRAYPWGAGAPWLVQW
YDGAGTKVEIK
B1 G2
(SEQ ID NO: 46)
TRVDQSPSSLSASVGDRVTITCVLTGANYGLASTYWYRKNPGSSNQERI
SISGRYSESVNKRTMSFTLTISSLQPEDSATYYCRAYPWGAGAPWLVQW
YDGAGTKVEIK
P3A1 V1
(SEQ ID NO: 47)
TRVDQSPSSLSASVGDRVTITCVLTDTSYGLYSTSWFRKNPGTTDWERM
SIGGRYVESVNKGAKSFTLTISSLQPEDFATYYCKAREARHPWLRQWYD
GAGTKVEIK
P3A1 G1
(SEQ ID NO: 48)
TRVDQSPSSLSASVGDRVTITCVLTDTSYGLYSTYWYRKNPGSSNKEQI
SISGRYSESVNKGTKSFTLTISSLQPEDSATYYCRAREARHPWLRQWYD
GAGTKVEIK
P3A1 G2
(SEQ ID NO: 49)
TRVDQSPSSLSASVGDRVTITCVLTDTSYGLYSTYWYRKNPGTTDWERM
SIGGRYSESVNKGAKSFTLTISSLQPEDSATYYCRAREARHPWLRQWYD
GAGTKVEIK
D3 humanised ADV1
(SEQ ID NO: 50)
ASVNQSPSSLSASVGDRVTITCVLTDTSYGLYSTSWFRKNPGTTDWERM
SIGGRYSESVNKGAKSFTLTISSLQPEDSATYYCKAQSGMAISTGSGHG
YNWYDGAGTKVEIK
D3 humanised ADV2
(SEQ ID NO: 51)
TRVDQSPSSLSASVGDRVTITCVLTDTSYGLYSTSWFRKNPGTTDWERM
SIGGRYSESVNKGAKSFTLTISSLQPEDSATYYCKAQSGMAISTGSGHG
YNWYDGAGTKVEIK
D3 humanised ADV3
(SEQ ID NO: 52)
ASVNQSPSSASASVGDRLTITCVLTDTSYGLYSTSWFRKNPGTTDWERM
SIGGRYSESVNKGAKSFTLTISSLQPEDSATYYCKAQSGMAISTGSGHG
YNWYDGAGTKLEVK
B1 humanised V5
(SEQ ID NO: 53)
ASVDQSPSSLSASVGDRVTITCVVTGANYGLAATYWYRKNPGSSNQERI
SISGRYSESVNKRTMSFTLTISSLQPEDSATYYCKAYPWGAGAPWLVQW
YDGAGTKVEIK
B1 humanised V7
(SEQ ID NO: 54)
ASVDQSPSSASASVGDRLTITCVVTGANYGLAATYWYRKNPGSSNQERI
SISGRYSESVNKRTMSFTLTISSLQPEDSATYYCKAYPWGAGAPWLVQW
YDGAGTKLEVK

DNA encoding the humanised constructs was codon optimised for expression in E. coli and synthesised by GeneArt (Thermo). P3A1 sequences were designed as dimers with a [G₄S]₅ linker connecting the VNAR domains. All humanised sequences were generated with the following C terminal His₆myc tag:

(SEQ ID NO: 97)
QASGAHHHHHHGAEFEQKLISEEDLG

DNA encoding these proteins was sub cloned into the intein expression vectors, expressed in E. coli and purified as described previously in “Typical method for expression of VNAR intein fusion proteins” section.

Further humanised versions of D3 were created as follows:

D3 humanised EL V1
(SEQ ID NO: 55)
ASVNQSPSSLSASVGDRVTITCVLTDTSYGLYSTSWFRKNPGTTDWERM
SIGGRYVESVNKRAKSFSLRIKDLTVADSATYYCKAQSGMAISTGSGHG
YNWYDGAGTKVEIK
D3 humanised EL V2
(SEQ ID NO: 56)
ASVNQSPSSLSASVGDRVTITCVLTDTSYGLYSTSWFRKNPGTTDWERM
SIGGRYVESVNKRAKSFTLTISSLQPEDFATYYCKAQSGMAISTGSGHG
YNWYDGAGTKVEIK
D3 humanised EL V3
(SEQ ID NO: 57)
ASVNQSPSSLSASVGDRVTITCVLTDTSYGLYSTSWFRKNPGTTDWERM
SIGGRFSGSGSKRAKSFTLTISSLQPEDFATYYCKAQSGMAISTGSGHG
YNWYDGAGTKVEIK
D3 humanised EL V4
(SEQ ID NO: 58)
ASVNQSPSSLSASVGDRVTITCVLTDTSYGLYSTSWYQQKPGTTDWERM
SIGGRYVESVNKRAKSFTLTISSLQPEDFATYYCKAQSGMAISTGSGHG
YNWYDGAGTKVEIK
D3 humanised EL V5
(SEQ ID NO: 59)
ASVNQSPSSLSASVGDRVTITCVLTDTSYGLYSTSWYQQKPGTTDWERM
SIGGRFSGSGSKRAKSFTLTISSLQPEDFATYYCKAQSGMAISTGSGHG
YNWYDGAGTKVEIK

Humanised ROR1 binding VNAR variants demonstrated high affinity binding to human ROR1 by SPR and improved thermal stability. SPR was performed as described previously using human ROR1 ECD—Fc immobilised to the chip surface. Thermal stability assays used Applied Biosystems StepOne Real Time PCR system with the Protein Thermal Shift™ dye kit (Thermo). The assay mix was set up so that the protein was at a final concentration of 20 μM in 20 μL. 5 μL of Thermal Shift™ buffer was added alongside 2.5 uL 8× Thermal Shift™ Dye. Assays were run using the StepOne software and data analysed using Protein Thermal Shift™ Software. All data are from first derivative analysis.

TABLE 8
Thermal stability and hROR1 binding
data for humanised VNAR variants

| | Tm | hROR1 binding (SPR) | | |
|---|---|---|---|---|
| Construct | (° C.) | Ka (M⁻¹s⁻¹) | Kd (s⁻¹) | K_{D} (nM) |
| B1 | 54.2 | 7.45E+05 | 6.09E−04 | 0.83 |
| B1G1 | 58.0 | 2.20E+05 | 1.62E−02 | 82.8 |
| B1G2 | 59.9 | 1.85E+05 | 7.90E−03 | 45.9 |
| B1V5 | 46.05 | 5.20E+04 | 3.85E−05 | 0.74 |
| B1V7 | 43.91 | 7.74E+04 | 5.54E−05 | 0.77 |
| P3A1 dimer | 60.7 | 3.78E+05 | 1.17E−03 | 0.30 |
| P3A1 V1 dimer | 48.5 | 4.78E+05 | 8.46E−04 | 0.18 |
| P3A1 G1 dimer | 57.1 | 4.30E+05 | 1.47E−03 | 0.43 |
| P3A1 G2 dimer | 54.0 | 1.88E+05 | 1.19E−03 | 0.77 |
| B1G1-hFc | ND | 2.4E+05 | 2.66E−03 | 11.8 |
| B1G2-hFc | ND | 6.26E+05 | 1.41E−03 | 2.55 |
| D3 ADV1 dimer | 54.66 | 6.36E+05 | 5.67E−03 | 8.92 |
| D3 ADV2 dimer | 56.18 | 6.09E+05 | 1.60E−02 | 26.2 |
| D3 WT | 64.4 | 1.21E+06 | 9.43E−05 | 15.5 |
| D3 AD V2 | 56.98 | 4.58E+04 | 2.36E−03 | 51.6 |
| D3 EL V1 | 53.25 | 1.50E+06 | 1.58E−04 | 16.1 |
| D3 EL V2 | 56.50 | 1.85E+06 | 1.63E−04 | 18.9 |
| D3 EL V4 | 54.1 | 1.38E+06 | 5.45E−04 | 58.5 |

Grafting the HV and/or CDR loops of B1 onto a humanised VNAR framework and substituting P3A1 sequences with regions from the human DPK-9 sequence, yielded substantially engineered proteins that are stable and maintain hROR1 binding with nanomolar and picomolar affinity respectively. Similar approaches yielded humanised variants of D3 that maintained similar binding characteristics as the wild type (WT) VNAR.

### Example 7—Epitope Mapping

### Binding of Proteins to Deglycosylated Human ROR1

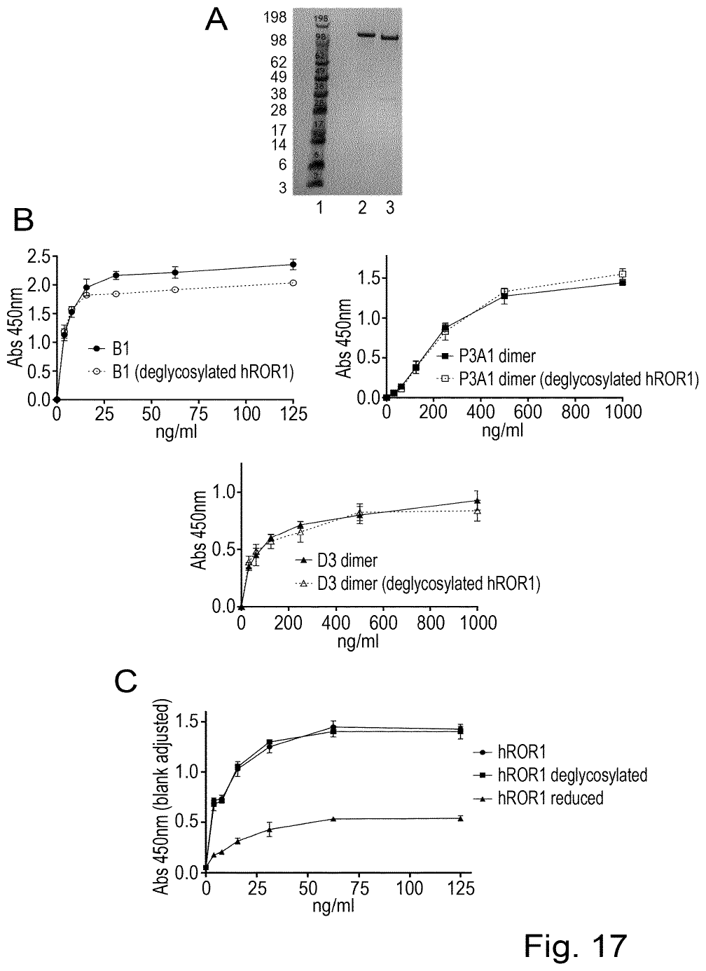

ELISA was used to compare VNAR binding to glycosylated and deglycosylated human ROR1 protein. To generate deglycosylated human ROR1, 0.2 mg/ml protein was incubated overnight at room temperature with 1 U PNGaseF (Roche) per 2 g ROR1 protein. Control, glycosylated human ROR1 was prepared in parallel without adding PNGaseF. SDS PAGE analysis showed shift on PNGaseF treatment, consistent with ROR1 deglycosylation (FIG. 17A).

These ROR1 proteins were used to coat ELISA plates and ELISAs were performed as previously described in the “Anti ROR1 VNAR characterisation” section. VNARs (B1, P3A1-P3A1, D3-D3, B1 mFc) bound equally well to both glycosylated and deglycoylated ROR1 proteins by ELISA (FIGS. 17B & 17C) indicating ROR1 binding is independent of ROR1 glycosylation.

Binding of B1 to unfolded hROR1 (reduced with 28 mM DTT, 0.5% Sarkosyl) was significantly reduced, consistent with B1 VNAR binding to conformational epitope(s) (FIG. 17C)

### Binding of B1 to ROR1 Ig Domain by SEC

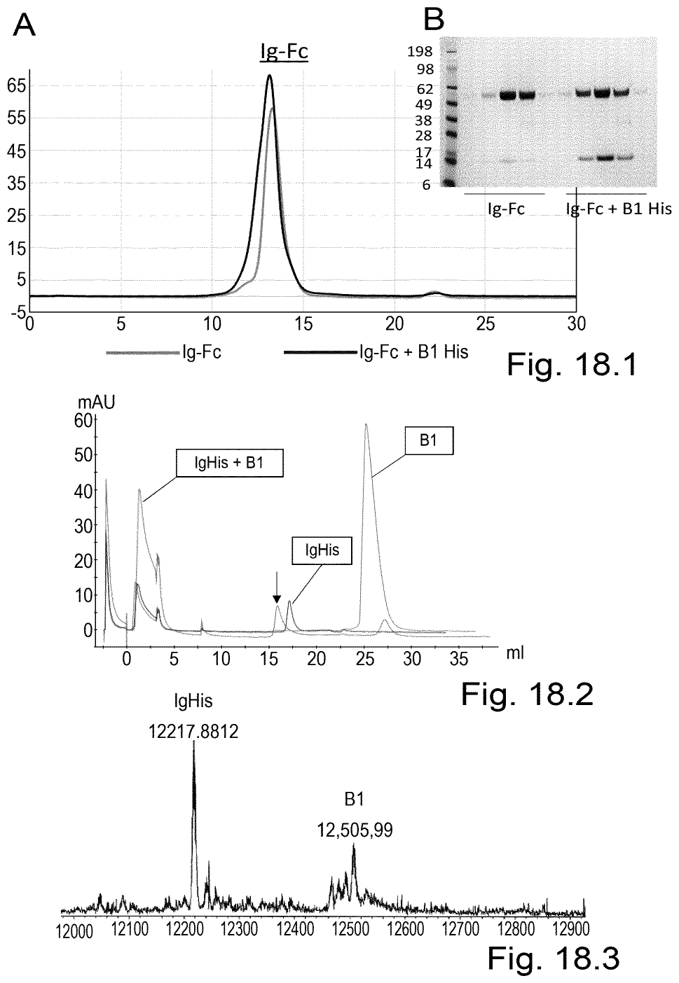

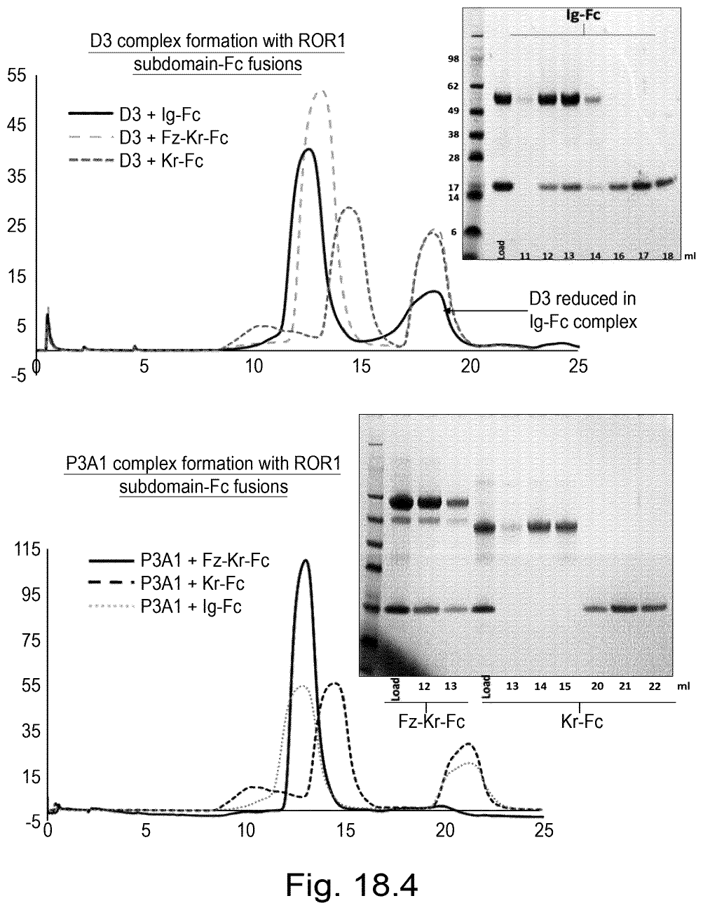

B1 VNAR forms a complex with ROR1 Ig domain by SEC (FIG. 18.1). 1:1 VNAR:ROR1 domain or ROR1 domain pairs was incubated on ice for 30 mins then run on a the Superdex 200 increase 10/300 column (GE Healthcare) in PBS and fractions analysed by SDS-PAGE. Under these conditions, B1 formed a complex with the ROR1 Ig domain.

### Binding of VNAR B1 to Non-Glycosylated ROR1 Ig Domain

In order to assess the involvement of glycosylation in the binding of VNAR B1 to ROR1, the ROR1 Ig domain was generated via expression in E. coli to produce a non-glycosylated form of the protein.

This non-glycosylated ROR1 Ig domain was then incubated on ice for 30 minutes with excess VNAR B1 (ratio 1:2) and assessed by size exclusion chromatography (SEC) using an analytical size exclusion column (S75 increase 10/300 GL analytical SEC column). Chromatography was carried out in 20 mM Hepes, 150 mM NaCl, pH7.5.

SEC analysis shows a new peak is formed eluting at a volume corresponding to a higher molecular weight species than ROR1 Ig or B1 alone (FIG. 18.2). MS analysis of this earlier elute peak shows that it corresponds to a complex between ROR1 Ig and B1 (FIG. 18.3).

### Further Domain Mapping

In order to determine the particular ROR1 domain to which individual VNARs bind, sub-domains and domain pairs of human ROR1 extracellular domain (ECD) were expressed as Fc fusion proteins. Complex formation was then assessed using analytical SEC.

Specifically, the following ROR1 domain fusions were created:
Ig-Fc
Ig-Fz-Fc
Fz-Kr-Fc
Fz-Fc
Kr-Fc
   Results of SEC analysis/SDS-PAGE are shown in FIG. 18.4 for (D3), and (P3A1). These data demonstrate that D3 binds to the ROR1 Ig domain, while P3A1 binds to either Fz alone or the Fz-Kr interface.

### Epitope Binning Experiments

Competition of binding studies were completed using SPR. Human ROR1 (hROR1) was immobilised to flow channels 1 and 3 (FC1 and FC3) of a COOH₂ chip by amine coupling. FC2 was used as the reference channel. A chosen VNAR e.g. B1, P3A1 dimer; or ROR1 2A2 mAb (BioLegend) was then captured to hROR1 on FC1. Test analytes were then assessed for binding to i) hROR1 with either VNAR or ROR1 2A2 mAb previously captured, or ii) to hROR1 in the absence of bound VNAR or mAb. The hROR1 chip surface was regenerated following each test analyte using Glycine pH2. Prior to testing the next analyte, VNAR or ROR1 2A2 mAb was again captured to hROR1 in FC1 and so on. Binding kinetics were determined using QDat software. For non-competing molecules, binding kinetics and sensogram profiles were similar/unaffected to hROR1 +/− captured binder. For competing molecules, the sensogram profile and binding kinetics were significantly altered.

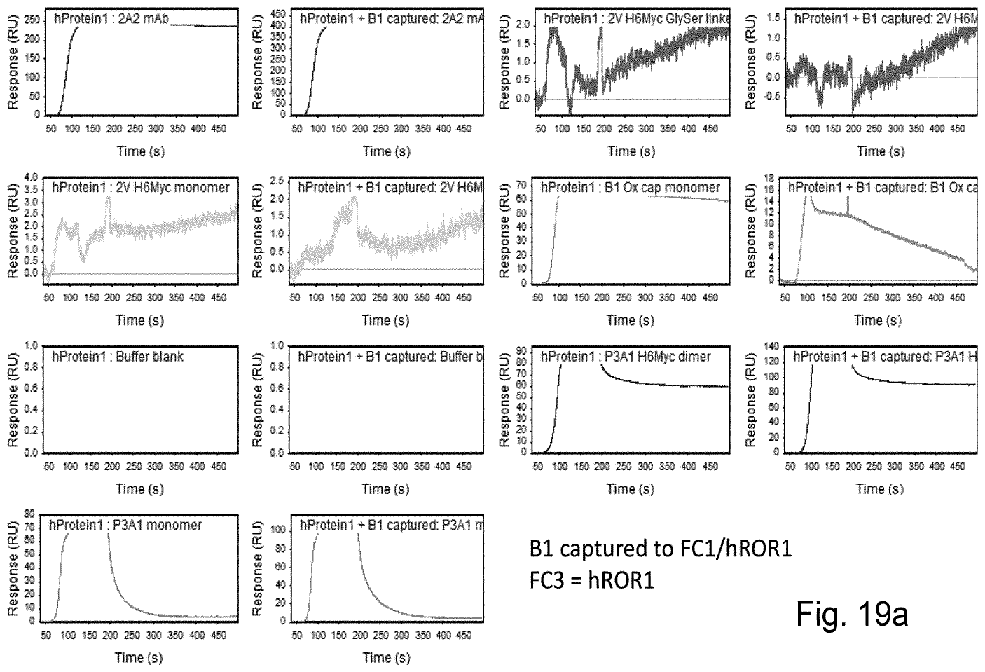

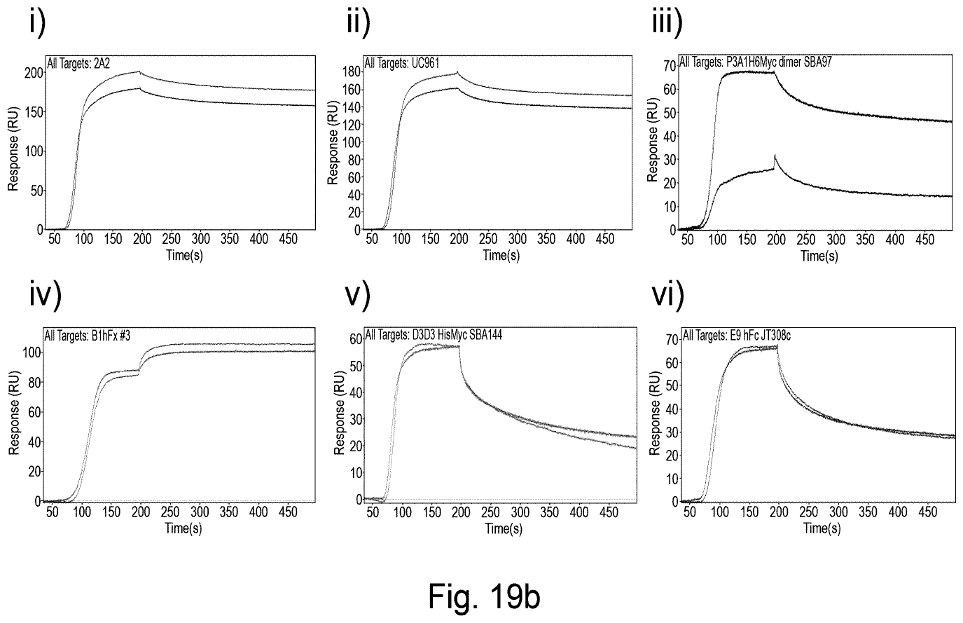

FIG. 19 shows representative sensograms and binding kinetics for binding of the VNARs to human ROR1 without and with prior incubation with B1. The results demonstrated that B1 and P3A1 VNARs do not compete with each other, nor with the ROR1 mAb 2A2 for binding to hROR1. When B1 VNAR was captured to hROR1 on the chip surface, further binding of B1 was significantly hindered, however the binding profiles of P3A1 monomer, P3A1 dimer or ROR1 2A2 mAb to hROR1 were the same in the absence and presence of pre-captured B1 (FIG. 19). The kinetic parameters derived for binding of these molecules to hROR1 in the presence or absence of captured B1 VNAR confirm that they do not compete with B1 (with the exception of B1, which competes with itself as expected).

TABLE 9
Binding kinetic data derived by SPR analysis of VNARs or ROR1
2A2 mAb to hROR1 +/− previously captured B1 VNAR. Data
demonstrates that B1 binding does not compete with P3A1 or 2A2.
VNARs were expressed with C-terminal His₆Myc tags.

| | hROR1 binding | | | B1 pre-captured tohROR1 | | |
|---|---|---|---|---|---|---|
| Mole- | Ka | Kd | KD | Ka | Kd | KD |
| cule | (M⁻¹s⁻¹) | (s⁻¹) | (nM) | (M⁻¹s⁻¹) | (s⁻¹) | (nM) |
| B1 | 1.04E+06 | 4.40E−04 | 0.424 nM | No/poor binding | | |
| P3A1- | 1.63E+06 | 6.28E−04 | 0.385 nM | 1.52E+06 | 5.36E−04 | 0.352 nM |
| P3A1 | | | | | | |
| P3A1 | 2.58E+06 | 4.11E−02 | 15.9 nM | 1.94E+06 | 3.20E−02 | 16.45 nM |
| ROR1 | 9.79E+05 | 2.11E−04 | 0.21 nM | 8.35E+05 | 8.47E−05 | 0.101 nM |
| 2A2 | | | | | | |
| mAb | | | | | | |
| (Bio- | | | | | | |
| legend) | | | | | | |

Binding of VNARs, 2A2 mAb or UC-961 based mAb to hROR1 with and without pre-capture of P3A1 derivatives were similarly assessed. The results are summarised in (FIG. 19b and Table 9b), which showed that P3A1 does compete with B, D3 or E9 or the mAb 2A2 or mAb based on UC-961 (by Kipps/Oncternal, Heavy Chain: SEQ ID NO:98; Light Chain: SEQ ID NO: 99). Table 9c further summarises the findings by SPR for competition of binding studies between the VNARs.

TABLE 9b
Binding kinetic data derived by SPR analysis of VNAR sequences,
UC961 based mAb or ROR1 2A2 mAb to hROR1 +/−
previously captured P3A1 His₆Myc dimer VNAR. Data
demonstrates that no competition of binding to hROR1
was observed between P3A1 and the other ROR1 binders,
other than P3A1 self-competition

| | | | | P3A1 dimer pre- | | |
|---|---|---|---|---|---|---|
| | hROR1 binding | | | captured to hROR1 | | |
| Mole- | Ka | Kd | KD | Ka | Kd | KD |
| cule | (M⁻¹s⁻¹) | (s⁻¹) | (nM) | (M⁻¹s⁻¹) | (s⁻¹) | (nM) |
| B1hFc | Strong binder, unable to | | | Strong binder, unable to | | |
| | determine Kd (off-rate) | | | determine Kd (off-rate) | | |
| P3A1 | 2.62E+06 | 1.41E−03 | 0.538 | 7.34E+05 | 2.26E−03 | 3.08 |
| dimer | | | | | | |
| D3 | 1.34E+06 | 3.62E−03 | 2.7 | 1.29E+06 | 4.47E−03 | 3.46 |
| dimer | | | | | | |
| E9hFc | 4.23E+05 | 3.45E−03 | 8.15 | 3.72E+05 | 3.74E−03 | 10.1 |
| UC961- | 5.77E+06 | 4.51E−04 | 0.078 | 5.48E+06 | 4.44E−04 | 0.081 |
| like | | | | | | |
| 2A2 | 1.47E+06 | 3.42E−04 | 0.233 | 1.39E+06 | 3.22E−04 | 0.231 |

TABLE 9c
Summary or results obtained using SPR to determine
competition of binding between molecules to hROR1.
P3A1 did not compete with any other sequences. B1, D3
and E9 exhibit competition of binding, suggesting
these sequences bind to overlapping epitopes of hROR1.
Competition of binding to hROR1 by SPR (Yes/No)

| Sequence | B1 | P3A1 | D3 | E9 |
|---|---|---|---|---|
| B1 | YES | NO | YES | YES |
| P3A1 | NO | YES | NO | NO |
| D3 | YES | NO | YES | YES |
| E9 | YES | NO | YES | YES |

### Epitope Mapping of Anti-ROR1 VNARs Using Anti-ROR1 Peptides

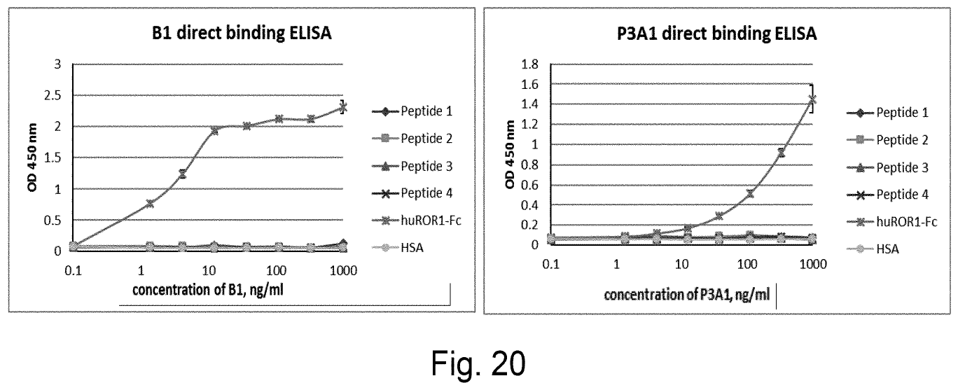

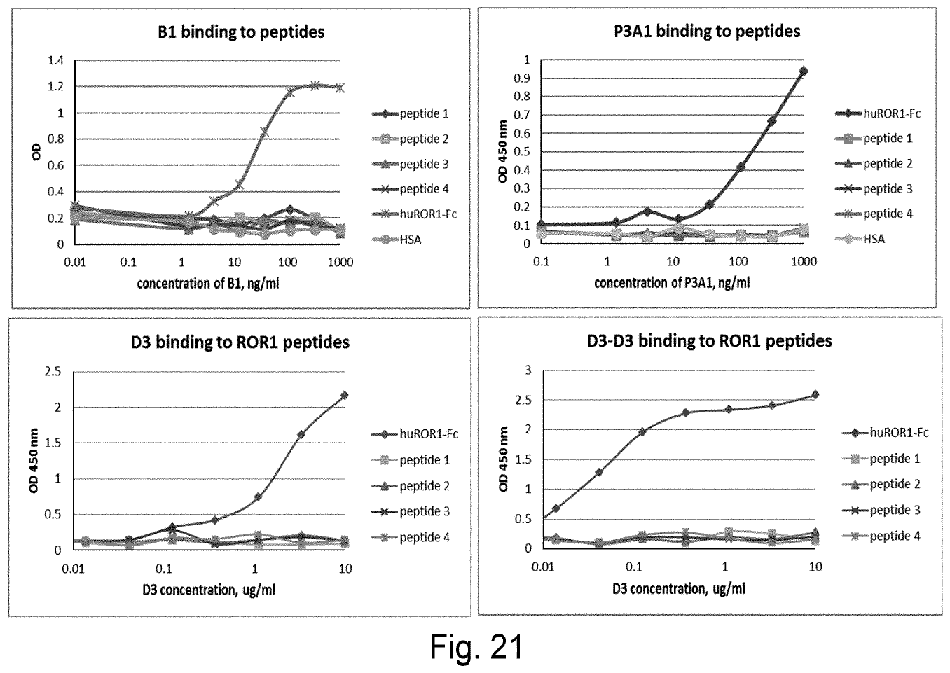

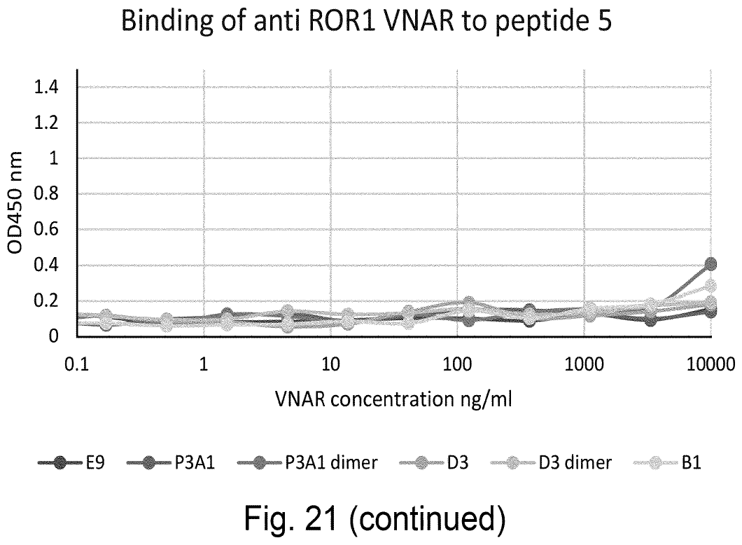

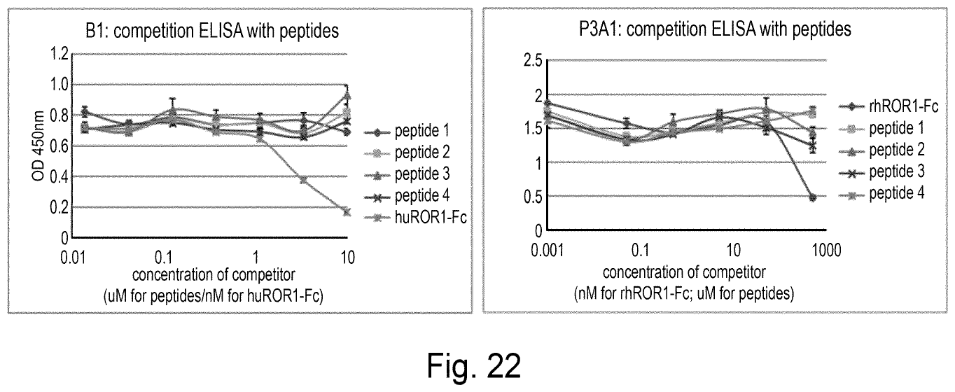

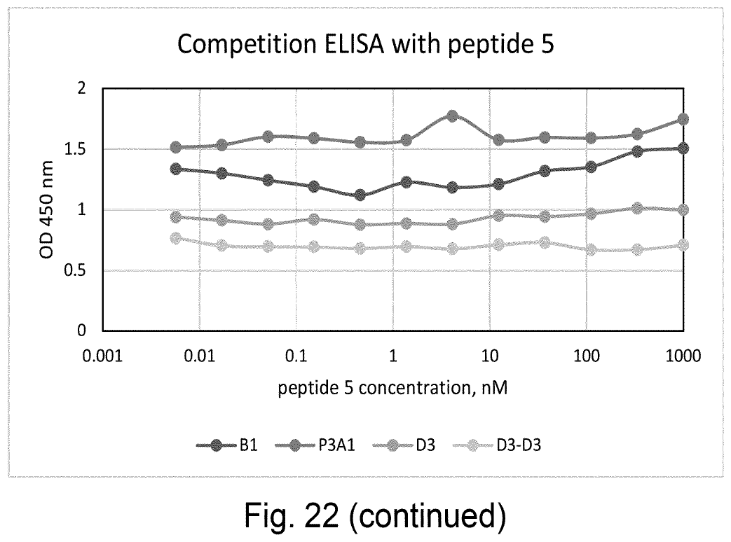

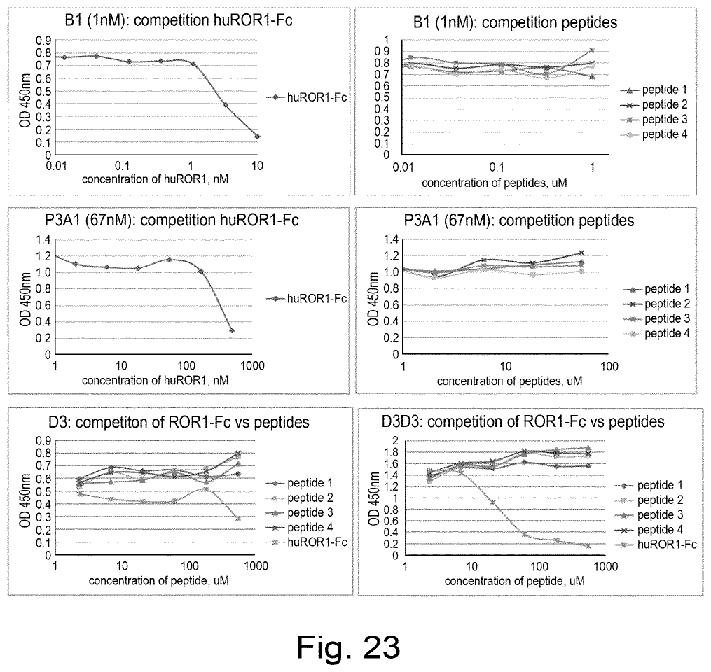

ELISA analysis was used to determine whether the lead anti-ROR1 VNAR domains, B1, P3A1 and D3 bound to the same or overlapping epitopes on ROR1 (defined here as four ECD peptides). Initial analysis of direct binding with peptides (in PBS and DMSO) immobilised onto ELISA plates indicated that none of the VNARs bound any of the peptides but did bind to the immobilised ECD hROR1-Fc protein control included as part of the same ELISA (FIG. 20 and FIG. 21). To interrogate this further, a competition assay was designed where VNARs were incubated with increasing concentrations of the four test peptides (or human ROR1 ECD-Fc) in solution and an assessment of residual binding to ROR1-Fc immobilised on an ELISA plate was then observed. Competition was evident between the VNARs and human ROR1 ECD-Fc, which was used as a positive control. However, no decrease signal was evident in the presence of the peptides, clearly indicating that no binding of VNAR to these specific ECD peptides had occurred (FIG. 22 and FIG. 23).

Further, B1, P3A1 and D3 VNARs do not bind any overlapping linear 15 mer peptides spanning the entire ECD of hROR1. Nor do they bind to hROR1 previously sonicated in SDS containing buffer under reducing conditions, conditions that typically denature protein (Pepscan data not shown). Together this indicates B1, P3A1 and D3 VNARs bind to distinct conformational epitope(s) on human ROR1 ECD protein.

### Direct Binding of VNARs to ECD Peptides

The following peptides were synthesised and dissolved in PBS pH 7.4:

Peptide 1-
(SEQ ID NO: 34)
YMESLHMQGEIENQI
Peptide 2-
(SEQ ID NO: 38)
RSTIYGSRLRINLDTTDTGYFQ
Peptide 3-
(SEQ ID NO: 35)
CQPWNSQYPHTHTFTALRFP
Peptide 4-
(SEQ ID NO: 37)
QCVATNGKEVVSSTGVLFVKFGPPPTASPGYSDEYE
Peptide 5-
(SEQ ID NO: 36)
RSTIYGSRLRIRNLDTTDTGYFQ

Clones B1 and P3A1 isolated from ELSS1 were assessed as monomers and D3 from an immunized library as both a monomer and a homodimer.

Both B1 and P3A1 demonstrated binding to ROR1 with no binding evident to any of the five peptides. HSA was included as a non-specific control (FIG. 20).

However as peptide 2 was insoluble in PBS, the direct binding ELISAs were repeated with the peptides dissolved in 25% DMSO. D3 and D3-D3 as a protein dimer fusion were included in these datasets and again no binding to the peptides was observed (FIG. 21).

### Methods

### Direct Peptide Binding ELISA

1. Coated 96 well plates with 10 or 50 nM huROR1-Fc in PBS or 10 μM of peptides in PBS or 25% DMSO. Incubated o/n at 4° C.
2. Washed 2×PBS
3. Blocked with 200 μl/well of 4% MPBS for 1 h at RT.
4. Washed 2×PBS
5. Added B1 or P3A1 at 1 μg/ml (67 nM); D3 and D3-D3 at 10 μg/ml (670 nM) and 1:3 serial dilutions across the plate. Incubated for 1 h at RT.
6. Washed 3×PBST
7. Incubated plates with 100 ul of anti-his-HRP SIGMA (1:1000 in PBST) for 1 h at RT
8. Washed 2×PBST and 2×PBS
9. Added 100 μl/well of TMB substrate. Stopped reaction with 1 M H2SO4

### Competition Assays of VNARs and ROR1 Peptides

Competition assays were conducted as described in the methods with all four peptides reconstituted in PBS. In these assays no binding was observed by VNARs B1 or P3A1 to any of the four peptides immobilised in typical binding ELISA format (FIG. 22). Therefore there was no evidence that these peptides represented epitopes on ROR1 that are recognised by B1 or P3A1.

Following the conditions used in FIG. 21 (due to peptide 2 being insoluble in PBS), all the competition assays were repeated with peptides dissolved in 25% DMSO. For the assay D3 and D3-D3 dimer were also included in these datasets. These results confirmed that the VNAR domains B1, P3A1 and D3 recognise a different epitope (or epitopes) from those represented by the 4 peptides tested.

### Methods

### Competition ELISA

1. Coated 96 well plates with 50 nM of huROR1-Fc for P3A1; 10 nM of huROR1-Fc for B1, D3 and D3-D3 dimer in PBS. Incubated o/n at 4° C.
2. Washed 2×PBS
3. Blocked with 200 μl/well 4% MPBS for 1 h at RT
4. Washed 2×PBS
5. Pre-incubated for 30 min at RT
B1=15 nM
Plus peptides (in PBS or 25% DMSO) at start concentration of 1 μM (then 1:3 serial dilutions across the plate)
or huROR1-Fc at start concentration of 100 nM (then 1:3 serial dilutions across the plate)
P3A1=670 nM
Plus peptides (in PBS or 25% DMSO) at starting concentration of 50 μM (then 1:3 serial dilutions across the plate)
or of huROR1-Fc at a starting concentration 1 μM (then 1:3 serial dilutions across the plate)
D3=67 nM
Plus peptides or huROR1-Fc (in PBS or 25% DMSO) at starting concentration of 500 nM (then 1:3 serial dilutions across the plate)
D3-D3=0.67 nM
Plus peptides or huROR1-Fc (in PBS or 25% DMSO) at starting concentration of 500 nM (then 1:3 serial dilutions across the plate)
6. Add 100 μl/well of pre-incubated samples. Incubated 1 h at RT
7. Washed 3×PBST
8. Incubated plates with 100 μl/well of anti-His-HRP (1:1000 in PBST). Incubated 1 h at RT
9. Washed 2×PBST and 2×PBS
10. Added 100 μl/well of TMB substrate. Stopped reaction with 50 μl/well 1 M H2SO4

### Epitope Mapping of Anti-ROR1 VNARs Using Recombinant ROR1 Domains

The ROR1 ECD is made up of three distinct protein domains: Ig-like, Frizzle and Kringle. To determine if the epitope recognised by each of these VNARs was within a specific sub-domain of the whole ROR1 protein the following ELISA analysis was performed.

### Direct Binding of VNARs to ROR1 Domains

Anti-ROR1 VNARs B1, P3A1 and D3 were assessed for binding to the three extracellular domains of human ROR1 (Ig-like, Frizzle and Kringle) by direct binding ELISA. B1 and P3A1 were assessed as monomers and D3 as both a monomer and a homodimer (D3-D3). 2A2 anti-ROR1 antibody was also incorporated into the assay as a positive control.

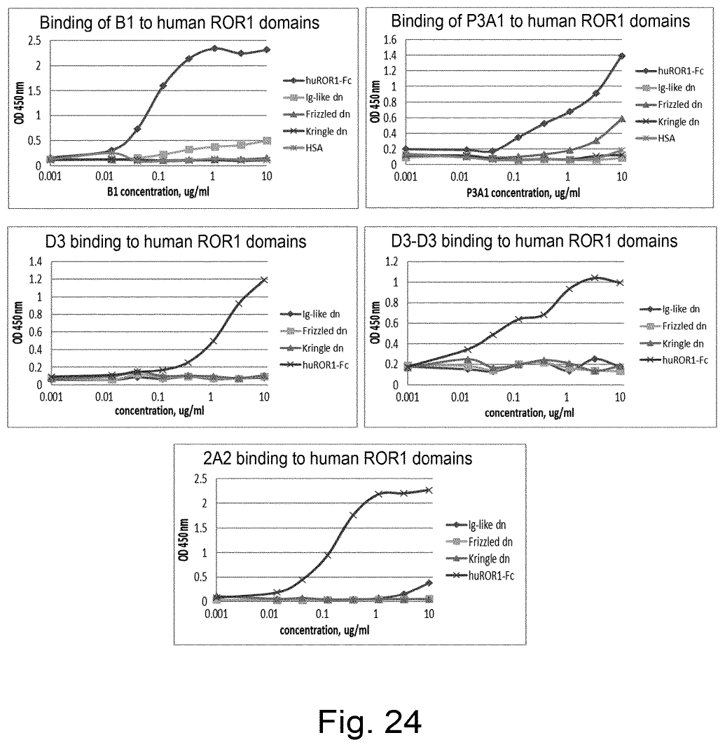

B1 and 2A2 recognised the Ig-like domain, however this binding to Ig-like domain was much weaker compared to their binding of the whole extracellular huROR1. P3A1 recognised the Frizzled domain but again weaker binding than to the intact ROR1 protein (FIG. 24 and Table 10). D3 and D3-D3 homodimer bound full length ROR1 ECD but no binding to individual ROR1 ECD sub domains was observed (FIG. 24 and Table 10).

All results are summarised in a Table 10.

TABLE 10

| | B1 | P3A1 | D3 | 2A2 |
|---|---|---|---|---|
| rhROR1-Fc | +++ | +++ | +++ | +++ |
| Ig-like domain | + | − | − | + |
| Frizzle domain | − | + | − | − |
| Kringle domain | − | − | − | − |

### Methods

### Direct Binding ELISA to ROR1 Domains

1. Coated 96 well plates with 1 μg/ml of huROR1-Fc or huROR1 domains in PBS. Incubated o/n at 4° C.
2. Washed 2×PBS
3. Blocked with 200 μl/well of 4% MPBS for 1 h at RT.
4. Washed 2×PBS
5. Added D3, D3-D3 dimer or 2A2 mAb at start concentration 10 μg/ml for VNAR and 1:150 dilution for mAb. Made 3-fold serial dilutions across the plate. Incubated for 1 h at RT.
6. Washed 3×PBST
7. Incubated plates with 100 μl of anti-c-myc-HRP (1:1000 in PBST) for 1 h at RT.
8. Washed 2×PBST and 2×PBS
9. Added 100 μl/well of TMB substrate. Stopped reaction with 1 M H2SO4.

### Example 8—VNAR Conjugation Chemistries

### Labelling of BA11 as Proof of Concept for Site-Specific VNAR Conjugation

Currently there are no methods for the site-specific conjugation of labels and drugs to VNARs, therefore there is a need to establish such conjugation methods. The VNAR BA11 is a humanised variant of E06 that binds with high affinity to human serum albumin (Kovalenko et al, J. Biol. Chem., 2013 JBC) and has applications as a half-life extension technology. BA11 was used as a model VNAR to determine whether site-specifically conjugated VNARs can be generated in good yield without compromising the binding activity of the VNAR domain. The C-terminus of VNARs is distal to the CDR1 & 3 and HV2 & 4 regions, which are the regions of the VNAR generally used to bind its target.

Therefore intein based technology (US2006247417) was used to assess the site-specific conjugation of payloads to the C-terminus of VNARs via different chemistries. Briefly, the protein of interest is expressed as an N terminal fusion of an engineered intein domain (Muir T W 2006 Nature 442, 517-518). Subsequent N to S acyl shift at the protein-intein union results in a thioester linked intermediate that can be chemically cleaved with bis-aminoxy agents or amino-thiols to give the desired protein C-terminal aminoxy or thiol derivative, respectively (FIG. 11). These C-terminal aminoxy and thiol derivatives can be reacted with aldehyde/ketone and maleimide functionalised moieties, respectively, in a chemoselective fashion to give the site-specific C-terminally modified protein (FIGS. 25-27). Using this approach BA11 fluorescein conjugates were generated via oxime and thioether forming chemistry in good yields and these conjugates maintained binding to human serum albumin protein.

Initially, the BA11 intein-CBD fusion protein, immobilised on chitin beads, was generated as described previously with typical yields 210 mg/L from cytosolic expression in E. coli. This precursor fusion protein was then cleaved under aqueous buffered conditions with different small molecule agents to generate BA11 with unique chemically reactive functionalities at its C-terminus.

### Generation of BA11-Aminoxy (FIG. 11)

Immobilised BA11 intein-CBD fusion protein was cleaved overnight in 400 mM dioxyamine (NH₂—O—(CH₂)₂—O—NH₂) in cleavage buffer pH6.9 resulting in ˜75% cleavage.

Cleavage supernatant containing BA11 aminoxy was drained and purified on a Superdex75 26/60 (GE Healthcare) in 20 mM sodium phosphate pH6.9, 200 mM NaCl. This yielded soluble, derivatised, folded protein with yields of >2 mg/L E. coli. All protein was characterised by reducing and non-reducing SDS PAGE analysis and mass spectrometry. The formation of the desired disulphide bond was confirmed by mass spec methods.

### Generation of BA11-Oxime-Fluorescein (FIG. 25)

Purified BA11 aminoxy was mixed with 3 molar equivalents benzaldehyde-peg-fluorescein in pH5.5 buffer with 10% acetonitrile and 10 mM aniline catalyst, room temperature overnight. SDS PAGE and mass spectrometry showed >98% reaction and conjugate was purified by SEC as above, and confirmed by reducing and non-reducing SDS PAGE analysis and mass spectrometry.

### Generation of BA11 C-Terminal Thiol Derivatives (FIG. 11)

BA11 intein-CBD fusion protein immobilised on chitin beads was cleaved overnight in 100 mM cysteamine (Sigma) in cleavage buffer with 2 mM TCEP to generate the corresponding C-terminal thiol derivative of the VNAR. The cleavage supernatant containing BA11 thiol was drained, treated with 2 mM TCEP to reduce any cysteamine adducts on the introduced C-term thiol group, and protein purified on a Superdex75 26/60 (GE Healthcare) in 20 mM sodium phosphate pH6.9, 200 mM NaCl. Yields ˜1.6 mg/L E. coli for BA11 SH were obtained. All proteins were characterised by reducing and non-reducing SDS PAGE analysis and mass spectrometry. The formation of the desired disulphide bond and free C-terminal thiol were confirmed by mass spec methods.

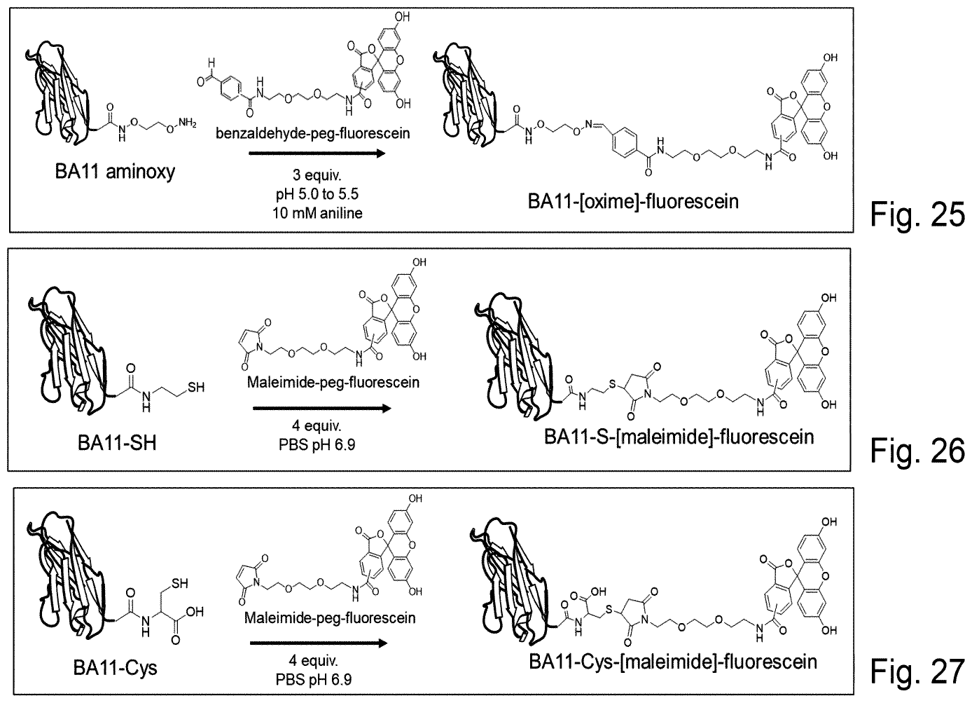

### Generation of BA11-C Term Thiol-Maleimide-Peg-Fluorescein (FIG. 26)

BA11 generated with a C-terminal thiol (BA11 SH) was mixed with 4 molar equivalents maleimide-peg-fluorescein in pH6.9 buffer with 0.3% DMF final, room temperature 0.5-1 hour. SDS PAGE and mass spectrometry showed >98% reaction. Conjugate was purified by SEC as above, and confirmed by reducing and non-reducing SDS PAGE analysis and mass spectrometry.

### Generation of BA11 C-Terminal Cysteine Derivatives (FIG. 11)

BA11 Intein-CBD fusion protein immobilised on chitin beads was cleaved overnight in 100 mM cysteine in cleavage buffer with 2 mM TCEP to generate the corresponding C-terminal cysteine derivative of the VNAR. The cleavage supernatant containing BA11 Cys was drained, treated with 2 mM TCEP to reduce any cysteine adducts on the introduced C-term thiol group, and protein purified on a Superdex75 26/60 (GE Healthcare) in 20 mM sodium phosphate pH6.9, 200 mM NaCl. Yields ˜>3 mg/L E. coli for BA11-cys were obtained. All proteins were characterised by reducing and non-reducing SDS PAGE analysis and mass spectrometry. The formation of the desired disulphide bond and free C-terminal cysteine thiol were confirmed by mass spec methods.

### Generation of BA11-C Terminal Cysteine-Maleimide-Peg-Fluorescein (FIG. 27)

BA11 generated with a C-terminal cysteine (BA11 cys) was mixed with 4 molar equivalents maleimide-peg-fluorescein in pH6.9 buffer with 0.3% DMF final, room temperature 0.5-1 hour. SDS PAGE and mass spectrometry showed 60-80% reaction for BA11 cys, lower reaction was due to significant BA11 cys dimer formation. Conjugate was purified by SEC as above, and confirmed by reducing and non-reducing SDS PAGE analysis and mass spectrometry.

The binding of BA11 and the corresponding C-terminal derivatives and conjugates to serum albumins was determined by SPR

### Determination of the Binding Kinetics of the Half-Life Extension VNAR (BA11) or Fluorescein-Conjugated-BA11 to Human, Mouse, Rat and Cynomolgous Serum Albumin

Binding kinetics were determined using SPR. The serum albumins or negative control protein were immobilised to COOH₂ chips by amine coupling using optimised buffer conditions as follows: Human serum albumin (HSA) and mouse serum albumin (MSA) were immobilised in sodium acetate pH5 buffer. Rat serum albumin (RSA) and cynomolgous serum albumin (CSA) in sodium acetate pH 4.5 buffer and the negative control hen egg lysozyme (HEL) protein was immobilised in sodium acetate pH 5.5 buffer.

Analytes (BA11, BA11-Fluorescein or 2V negative control binder) were tested at various concentrations and the Ka (M⁻¹s⁻¹), K_{d} (s⁻¹) and K_{D} (nM) values were determined using QDat software (SensiQ/Pall ForteBio). For each analyte test experiment, binding to the chosen serum albumin protein was assayed alongside the negative control protein (HEL).

TABLE 11
Summary of SPR data (K_{D} nM) for BA11 C terminal
derivatives and subsequent fluorescein conjugates with
different conjugation chemistries binding to serum albumin proteins.

| | Serum Albumin | | | | | | | |
|---|---|---|---|---|---|---|---|---|
| | Human | | Rat | | Mouse | | Cyano | |
| | pH | | | | | | | |
| | 7.4 | 5.5 | 7.4 | 5.5 | 7.4 | 5.5 | 7.4 | 5.5 |
| BA11 | 0.636 | 0.910 | 2.969 | 4.389 | 1.902 | 4.804 | 3.360 | 1.109 |
| BA11- | 1.118 | ND | 10.85 | ND | 8.767 | 21.20 | 7.970 | ND |
| aminoxy | | | | | | | | |
| BA11- | 0.677 | 1.296 | 5.725 | 5.928 | 4.238 | 7.442 | 1.748 | 5.540 |
| oxime-Fl | | | | | | | | |
| BA11-cys | 0.756 | 1.956 | 3.370 | 3.215 | ND | 3.775 | 2.103 | ND |
| BA11-cys- | 1.097 | 3.160 | 4.775 | 7.240 | 5.064 | 13.645 | 3.681 | 8.205 |
| mal-Fl | | | | | | | | |
| BA11-SH | 0.774 | 2.229 | 7.671 | 12.10 | 4.764 | 11.08 | 3.414 | 6.738 |
| BA11-S- | 1.417 | 1.912 | 5.297 | 7.925 | 5.004 | 10.60 | 2.300 | 7.010 |
| mal-Fl | | | | | | | | |

| | |
|---|---|
| 2V | Did not bind |

Fl, fluorescein;
cys, cysteine;
mal, maleimide;
SH, thiol;
2V, non-binding VNAR negative control.

All BA11 derivatives and conjugates showed high affinity binding to the different serum albumin proteins at both pH7.4 and pH5.5. Therefore the methodologies described provide robust high yielding approaches for the site-specific modification and conjugation of VNARs that maintain the binding activity of the protein.

### ROR1 Binding VNARs—AF488 and MMAE Conjugates

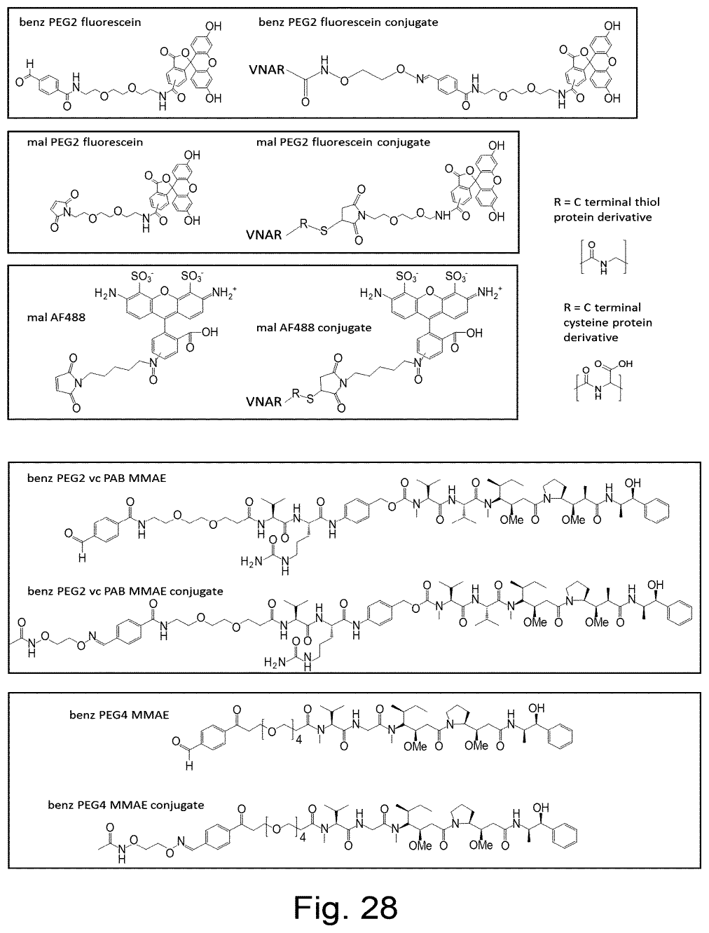

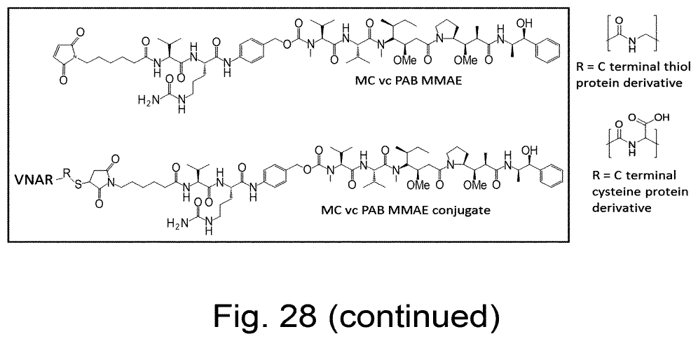

Expression of ROR1 binding VNARs as C-terminal intein fusion proteins enabled generation of ROR1 binding VNARs with unique C-terminal aminoxy and C-terminal thiol groups. This in turn enabling site specific, C-terminal conjugation to fluorescent labels and cytotoxic payloads via oxime forming conjugation chemistry and maleimide chemistry, respectively. Examples of labels and payloads used are shown in FIG. 28.

ROR1 binding VNAR intein CBD fusion protein immobilised on chitin beads was generated as described above

### Generation of VNAR-ox-vcMMAE and VNAR-ox-MMAE

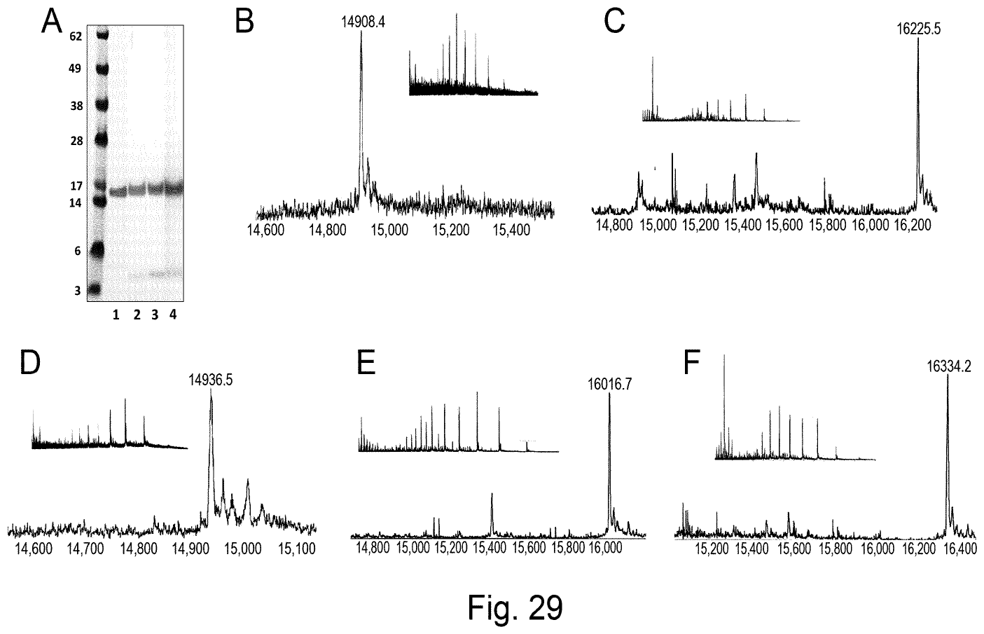

The immobilised VNAR intein fusion protein was cleaved with 400 mM O,O′-1,3-propanediylbishydroxylamine (NH₂—O—(CH₂)₃—O—NH₂) in cleavage buffer pH 6.9, room temperature overnight. The resulting VNAR containing a C-terminal aminoxy group (VNAR aminoxy) was purified by IMAC or SEC and reacted with 3 molar equivalents of benzaldehyde PEG2 vc PAB MMAE or benzaldehyde PEG4 MMAE in 10% acetonitrile with 10 mM aniline catalyst final, room temperature overnight. Conjugates were purified by IMAC or SEC, sterile filtered and formation of the desired material and final purity confirmed by reducing and non-reducing SDS PAGE analysis and mass spectrometry (FIG. 29)

### Generation of VNAR-S-Mal-vcMMAE

The immobilised VNAR intein fusion protein was cleaved with 100 mM cysteamine in cleavage buffer pH 6.9 with 2 mM TCEP, room temperature overnight. The resulting VNAR containing a C-terminal thiol group (VNAR SH) was purified by IMAC or SEC and reacted with 4 molar equivalents of MC vc PAB MMAE or malAF488. Conjugates were purified by IMAC or SEC, and sterile filtered and formation of the desired material and final purity confirmed by reducing and non-reducing SDS PAGE analysis and mass spectrometry (FIG. 29)

### Characterisation of Anti ROR1 VNAR-MMAE Conjugates—Binding to ROR1 and ROR2 by SPR and Cell Surface Binding by Flow Cytometry

### Binding of VNAR Conjugates to ROR1 and ROR2 by SPR

The ability of the VNAR-MMAE conjugates and VNAR-fluorescein conjugates to bind to human ROR1 ECD was determined by SPR using the procedures described above.

As shown in Table 12 VNAR conjugates that were prepared through oxime ligation of benzaldehyde payloads to C-terminal aminoxy VNARs; through thioether ligation of malemide functionalised payloads to C-terminal thiol VNARs and through thioether ligation of malemide functionalised payloads to C-terminal Cysteine VNARs all maintain high affinity for human ROR1 but do not bind to human ROR2. Conjugates were prepared using enzyme cleavable linkers (Val-Cit) or non-cleavable linkers and showed similar binding to human ROR1.

TABLE 12
SPR data for binding of VNARs and corresponding Fluorescein
and MMAE conjugates to human ROR1 and ROR2.
VNARs were expressed with C-terminal His₆Myc tags.

| | hROR1 | | | |
|---|---|---|---|---|
| VNAR | Ka (M⁻¹s⁻¹) | Kd (s⁻¹) | KD (nM) | hROR2 |
| B1 | 4.75E+05 | 7.56E−04 | 1.65 | No binding |
| B1-S-mal- | 4.7E+05 | 3.67E−04 | 0.81 | No binding |
| Fluorescein | | | | |
| 2V | No binding | No binding | No binding | No binding |
| 2V-S-mal- | No binding | No binding | No binding | No binding |
| vcMMAE | | | | |
| 2V-Ox-vcMMAE | No binding | No binding | No binding | No binding |
| 2V-Ox-MMAE | No binding | No binding | No binding | No binding |
| P3A1-P3A1 | 1.86E+06 | 2.96E−03 | 1.61 | No binding |
| P3A1-P3A1-S- | 4.96E+06 | 2.6E−03 | 0.59 | No binding |
| mal-vcMMAE | | | | |
| P3A1-P3A1-Ox- | 2.07E+06 | 2.77E−03 | 1.43 | No binding |
| vcMMAE | | | | |
| P3A1-P3A1-Ox- | 4.20E+06 | 3.20E−03 | 0.78 | No binding |
| MMAE | | | | |
| 2V-2V | No binding | No binding | No binding | No binding |
| 2V-2V-S-mal- | No binding | No binding | No binding | No binding |
| vcMMAE | | | | |
| 2V-2V-Ox- | No specific | No specific | No specific | No binding |
| vcMMAE | binding | binding | binding | |
| 2V-2V-Ox-MMAE | No binding | No binding | No binding | No binding |

### Binding of VNAR Conjugates to Cancer Cell-Lines

Binding of B1 and P3A1 MMAE conjugates to cancer cell-lines was determined by flow cytometry using methods described above. B1 and P3A1 conjugates maintain binding to the ROR1^{hi} A549 lung adenocarcinoma cells and do not bind the ROR1^{low} lung cancer cell-line A427 by flow cytometry at a fixed concentration of protein.

### VNAR mFc Fusion Protein Conjugates

B1 mIgG2a Fc and nonbinding 2V mIgG2a Fc fusion proteins were labelled with mal AF488 and mc vc PAB MMAE via protocols adapted from the partial reduction and labelling of antibody interchain disulfides (Methods in Molecular Biology vol 1045 chapter 9; Sun et al, Bioconj Chem 2005). Briefly VNAR mIgG2a Fc proteins at 1 mg/ml in PBS+100 mM L-Arg with 1 mM EDTA added were partially reduced with 2.75 molar equivalents fresh TCEP; 37° C. 2 hours. 1.1 molar equivalents maleimide label to free protein thiol was added, incubated on ice 45 mins and L-cysteine added to stop the reaction. Reactions were dialysed to remove unreacted label/drug, sterile filtered and analysed by SDS PAGE. Typical DAR of 4.4 for B1-mFc-AF488, and 3.9 for 2V-mFc-AF488.

### Vnar hFc Fusion Protein Drug Conjugates

Another approach for generating ADCs is to engineer cysteine substitutions or additions at positions on the light and heavy chains of antibodies and these cysteines provide reactive thiol groups for site specific labelling (Junutula 2008 Nature Biotechnology 26, 925-932, Jeffrey 2013, Sutherland 2016).

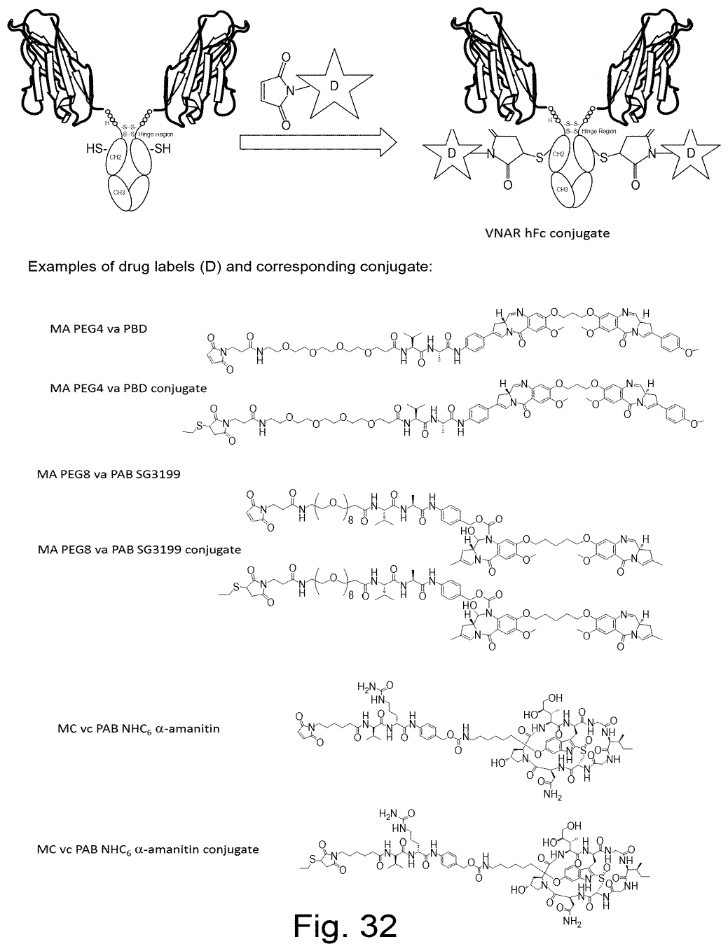

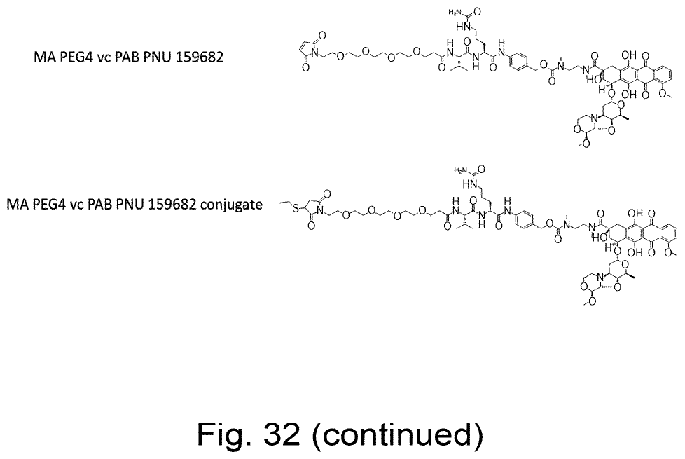

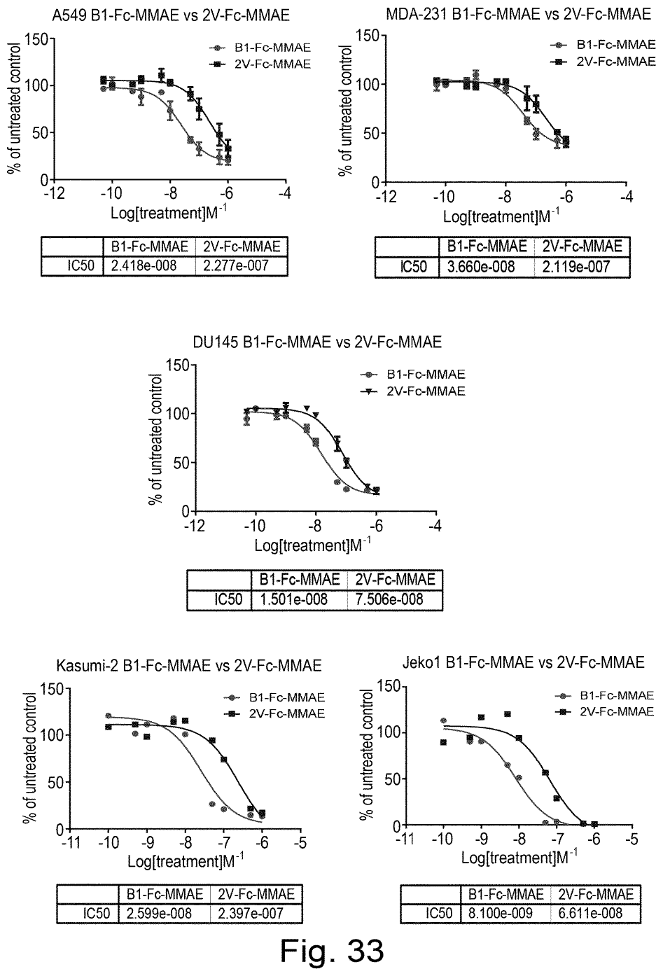

Anti ROR1 VNARs were genetically fused to engineered hIgG1 Fc domains that contained a cysteine substitution in the hIgG1 Fc sequence, S252C or S473C (Kabat numbering). This enabled site specific labelling with maleimide derivatives of fluorescent labels (AF488) and cytotoxic drugs (MC vc PAB MMAE, MC vc PAB NHC₆ α-amanitin, MA PEG4 va PBD, MA PEG8 va PAB SG3199, MA PEG4 vc PAB DMAE PNU 159682) (FIG. 32).

### Generation of VNAR-hFc—Drug Conjugates

A partial reduction, refolding and labelling method to label the VNAR Fc S252C or VNAR Fc S473 was adapted from the literature (Junutula et al, 2008 Nat Biotech, Jeffrey et al, 2013 Bioconj Chem). Briefly, 1 mg/ml VNAR hFc solutions were prepared in PBS+100 mM L-Arginine pH7.4 with 1 mM EDTA. 20 molar equivalents TCEP added and incubated at 4° C. for a minimum of 48 hours. 30 molar equivalents DHAA added, pH adjusted to 6.5 and incubated at room temperature for 1 hour. Refolded VNAR Fc S252C or S473C was extensively dialysed or buffer exchanged into PBS+50 mM L-Arginine and quantified by UV before reacting with 4 molar equivalents maleimide label/drug solution, room temperature 1 hour to overnight depending on label/drug. Conjugates were dialysed/buffer exchanged directly or purified further by SEC or IEX before dialysis/buffer exchange.

This approach was used to generate MMAE conjugates of B1, P3A1 and 2V Fc fusion proteins whereby the corresponding hIgG1 Fc (S252C or S473C) derivative was labelled with a maleimide functionalised MMAE payload incorporating an enzyme cleavable (Cathepsin B) linker.

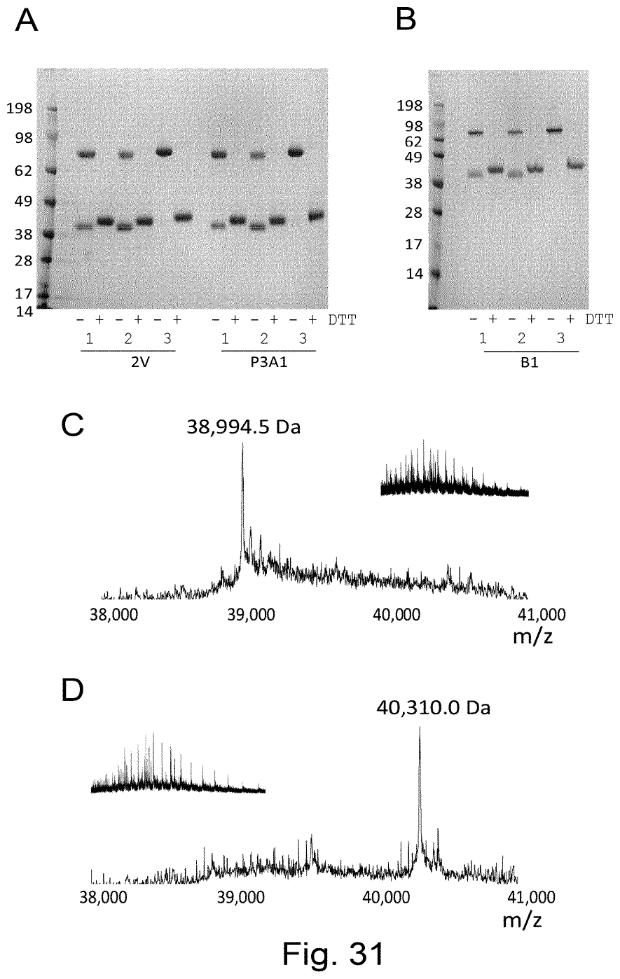

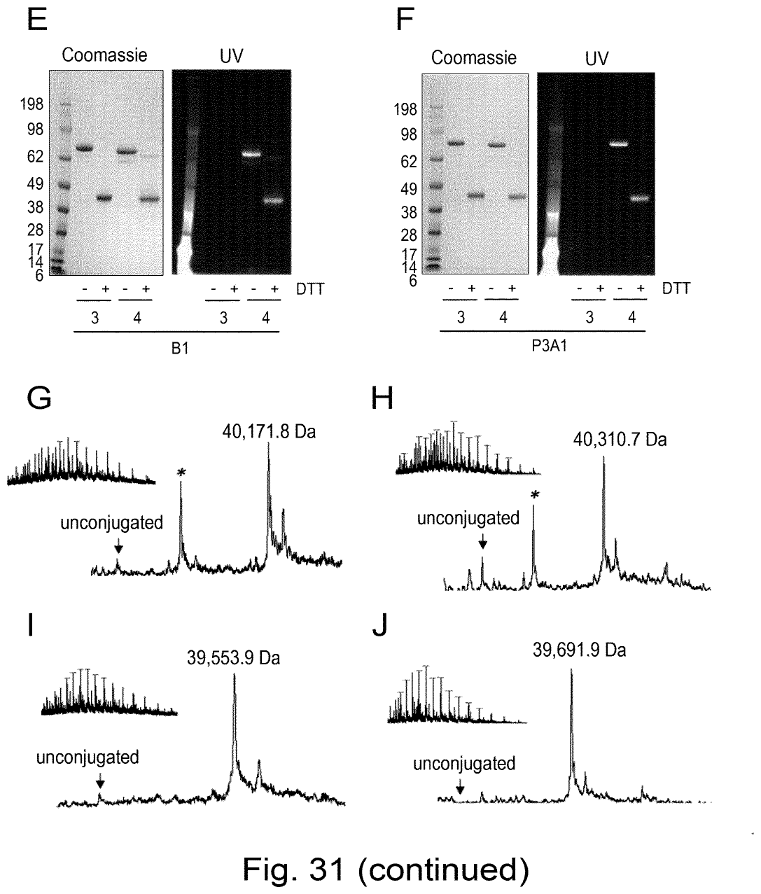

SDS-PAGE and mass spectrometry analysis of the final conjugates determined that the labelling had proceeded in a quantitative fashion to give highly pure homogenous VNAR-hFc-MMAE conjugates with drug to antibody ratio (DAR) of 2 (FIG. 31 shows conjugation to VNAR-hFc(S252C). Similar procedures were used to generate PBD dimer, α-amanitin and PNU conjugates of cysteine engineered VNAR-hFc fusion proteins (Levena Biopharma, San Diego). Whereby VNAR (B1, P3A1, 2V) hIgG1 Fc(S252C) fusions were reacted with MC vc PAB NHC₆ α-amanitin, MA PEG4 va PBD, MA PEG8 va PAB SG3199, MA PEG4 vc PAB DMAE PNU 159682 (FIG. 32).

Binding of VNAR-hFc-MMAE Conjugates to hROR1 and Cancer Cell-Lines

The ability of the VNAR-hFc conjugates to bind to human ROR1 ECD was determined by SPR using the procedures described above.

TABLE 13
SPR data for binding of VNAR human Fc (hFc) and MMAE
conjugated versions to human ROR1 and human ROR2

| | hROR1 | | | |
|---|---|---|---|---|
| Molecule set | Ka (M⁻¹s⁻¹) | Kd (s⁻¹) | KD (nM) | hROR2 |
| B1 hFc | 3.08E+06 | 9.53E−05 | 0.032 | No binding |
| B1 hFc-MMAE | 1.22E+06 | 1.29E−04 | 0.105 | No binding |
| P3A1 hFc | 1.07E+07 | 5.64E−04 | 0.084 | No binding |
| P3A1 hFc-MMAE | 2.68E+06 | 1.00E−03 | 0.38 | No binding |
| 2V hFc | No binding | No binding | No binding | No binding |
| 2V hFc-MMAE | No binding | No binding | No binding | No binding |
| 2V-2V hFc | No binding | No binding | No binding | No binding |

B1 and P3A1 VNAR-hIgG Fc (S252C)-vcMMAE conjugates demonstrated high affinity binding to ROR1 but do not bind to human ROR2. 2V is a non-binding VNAR and the corresponding 2V-hFc drug conjugates were generated as non-binding controls.

Binding of B1 and P3A1 hFc-vcMMAE conjugates to ROR1^{hi} A549 lung adenocarcinoma cell-line and the ROR1^{low} A427 lung cancer cell-line was determined by flow cytometry using methods described above.

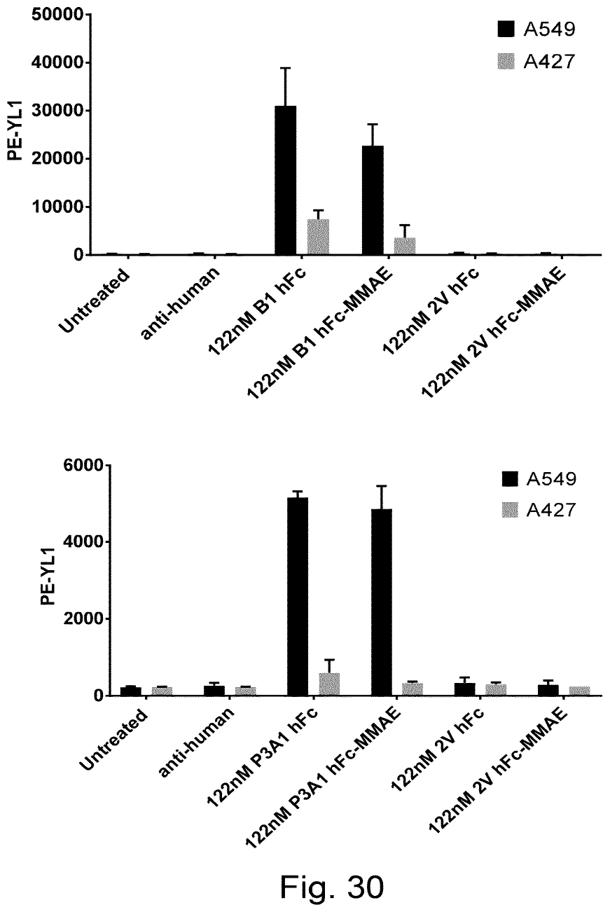

FIG. 30 shows that B1 and P3A1 hFc-vcMMAE conjugates bind strongly to the ROR1 hi cancer cells but not the ROR1^{low} cancer cells. Whilst the 2V-hFc-vcMMAE conjugate does not bind to either cell-line.

In Vitro Cell Viability Assays for Cancer Cells Treated with Anti ROR1 VNAR Drug Conjugates

Cells were seeded into white, clear bottom 96 well plates (Costar) and incubated at 37° C., 5% CO2 for 24 hours. On the following day, dilution series were set up for each test agent at ×10 working stocks. The dose response ×10 stock was: 10000, 5000, 1000, 500, 100, 50, 10, 5, 1, 0.5 nM. 10 μL of the ×10 stock solutions were added to the cell plates (90 μl per well) using a multichannel pipette. This resulted in a 1:10 dilution into the well and dose responses ranging from 1000 nM (column 1) to 0.05 nM (column 10). 10 μl of vehicle control (PBS) was added to the control wells (columns 11 and 12). Plates were incubated at 37° C., 5% CO2 for 72-96 hours. Promega Cell Titre Glo reagent was used as per the manufacturer's instructions to assess cell viability. Briefly, assay plates were removed from incubator and allowed to equilibrate to room temperature before adding 100 μl of room temperature Cell Titre Glo reagent to each 100 μl assay well. Plates were placed on a plate shaker for 2 minutes at 600 rpm. Plates were allowed to sit for a further 10 minutes at room temperature prior to measuring luminescence read-out using a Clariostar plate-reader (BMG). Data was analysed by calculating the average for untreated (vehicle only) control wells and determining the % of control for each treated well. % of control data was then plotted against Log [Treatment] concentration and the IC₅₀ value derived using non-linear regression fitting in GraphPad Prism software.

### Cell Lines

DU145 prostate cancer cells: EMEM, 10% hiFCS
JeKo-1 Mantle cell lymphoma cells: RPMI 1640, 20% hiFCS
Kasumi-2 B cell precursor leukemia cells: RPMI 1640, 10% hiFCS
PA-1 ovarian cancer cells: EMEM, 10% hiFCS
PA-1 ROR1 knockout cells: EMEM, 10% hiFCS
A549 cells: DMEM, 10% hiFCS
MDA-MB-231 cells: DMEM, 10% hiFCS

FIG. 33 shows dose response curves, with corresponding IC50 values, for cell-killing of the ROR1 positive cancer cell-lines A549 (lung adenocarcinoma), MDA-MB-231 (breast cancer), DU145 (prostate cancer), Kasumi-2 (ALL cells) and Jeko1 (MCL cells) by B1-mFc-vcMMAE and 2V-mFc-vcMMAE conjugates. B1-mFc-vcMMAE conjugates show potent cell-killing of the ROR1 positive cancer cells and show superior potency to the corresponding 2V-mFc-vcMMAE conjugate across each of the cell-lines.

TABLE 14
IC50 values for cell-killing by B1-mFc-MMAE and
2V-mFc-MMAE per cell line.

| | IC50 (nM) | |
|---|---|---|
| Cell line | B1 mFc MMAE | 2V mFc MMAE |
| A549 | 24.2 | 228 |
| MDA-231 | 36.6 | 212 |
| DU145 | 15 | 75 |
| Kasumi-2 | 26 | 240 |
| JeKo-1 | 8.1 | 66 |

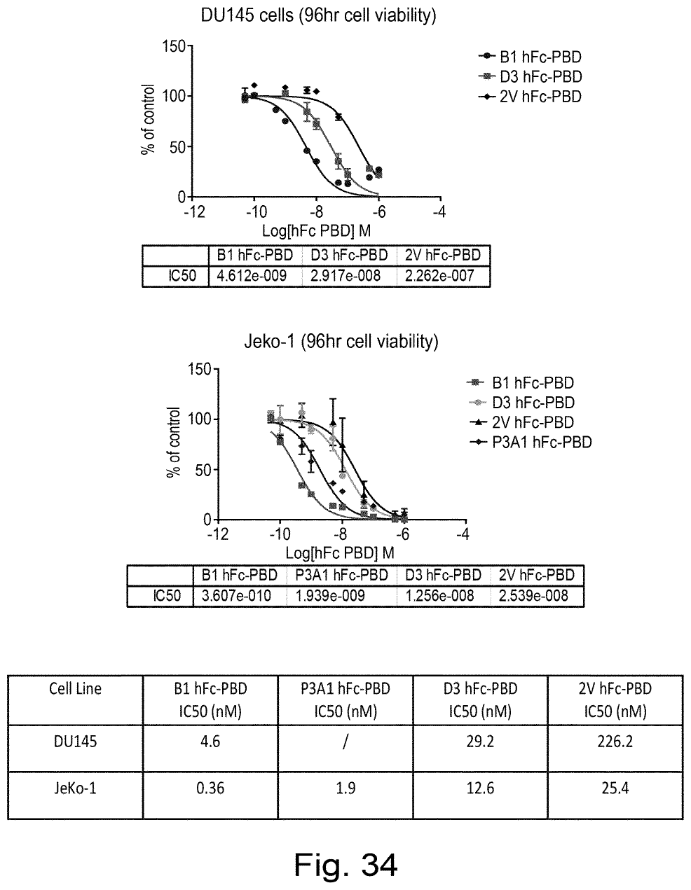

FIG. 34 shows dose response curves, with corresponding IC50 values, for cell-killing of A) the ROR1 positive DU145 prostate cancer cells by B1-hFc-PBD, D3-hFc-PBD and 2V-hFc-PBD conjugates and B) ROR1 positive Jeko1 MCL cells by B1-hFc-PBD, P3A1-hFc-PBD, D3-hFc-PBD and 2V-hFc-PBD conjugates.

TABLE 15
IC50 values (nM) determined for VNAR hFc-PBD molecules
in DU145 and Jeko-1 cancer cell lines at 96 hr.

| | IC50 (nM) | | | |
|---|---|---|---|---|
| Cell Line | B1 hFc-PBD | P3A1 hFc-PBD | D3 hFc-PBD | 2V hFc-PBD |
| DU145 | 4.6 | / | 29.2 | 226.2 |
| JeKo-1 | 0.36 | 1.9 | 12.6 | 25.4 |

The ROR1 targeting VNAR-PBD conjugates show potent killing of both cancer cell-lines and show increased potency with respect to the 2V-hFc-PBD conjugate, with the IC50 values for the B1-hFc conjugate at least 49 fold lower than 2V-hFc conjugate.

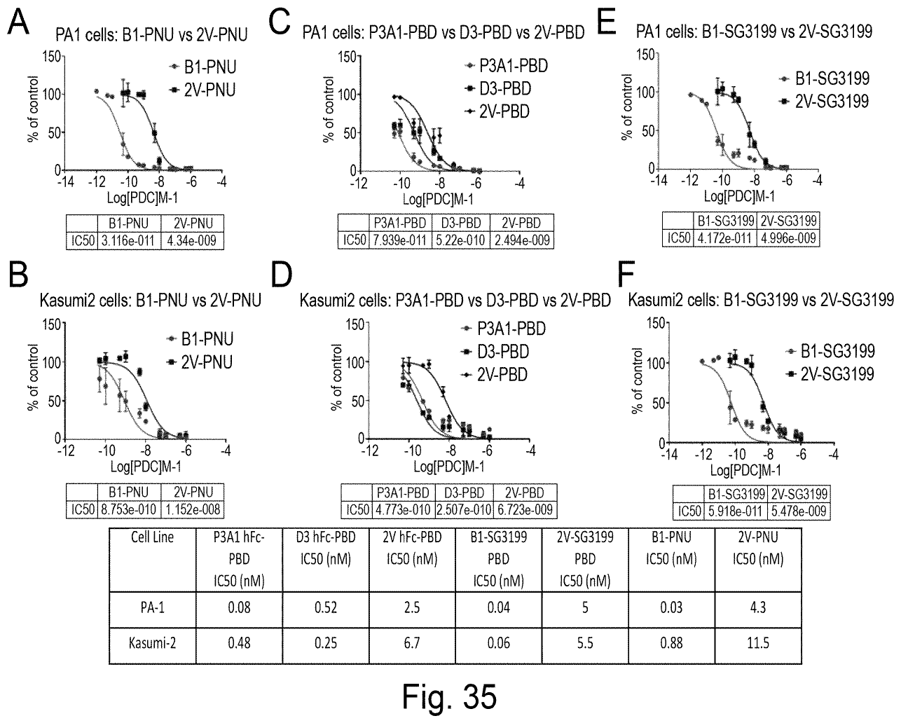

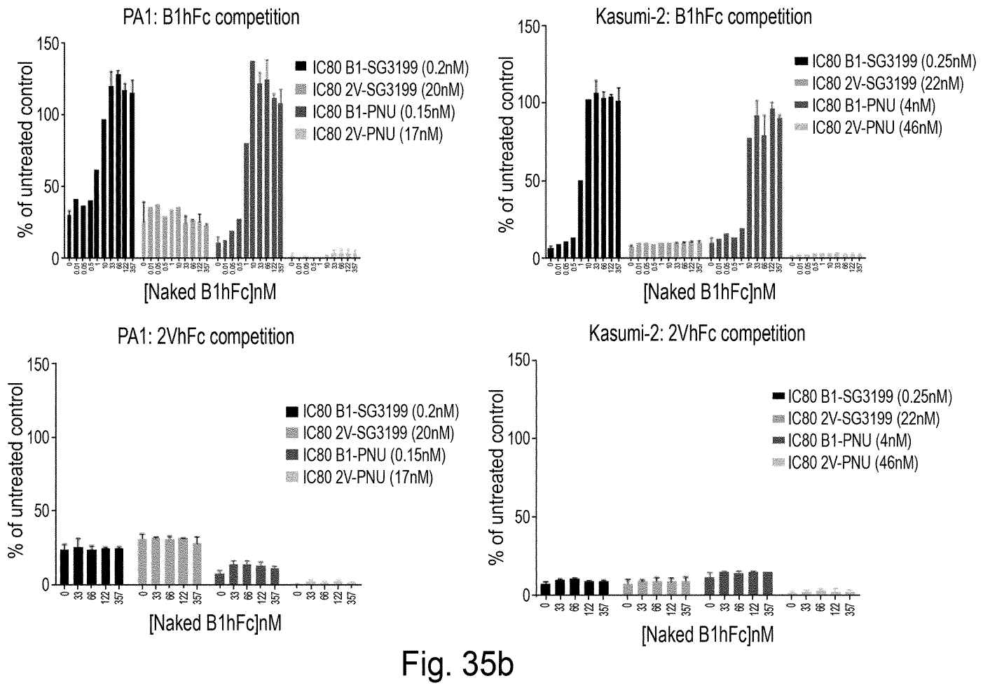

FIG. 35 shows dose response curves, with corresponding IC50 values, for cell-killing of the ROR1 positive PA-1 ovarian cancer cells (A, C, E) and Kasumi-2 B-cell precursor leukaemia cells (B, D, F) by B1-hFc-PNU, 2V-hFc-PNU conjugates (PEG4-vc PAB DMAE PNU 159682), P3A1-hFc-PBD, D3-hFc-PBD and 2V-hFc-PBD conjugates and B1-hFc SG3199 PBD and 2V-hFc SG3199 PBD conjugates.

TABLE 16
Calculated IC50 values (nM) for the cell-killing of PA-1 and Kasumi-2
cancer cells by VNAR-hFc conjugates. PA-1 ROR1 ko is PA-1 cancer
cell-line where ROR1 expression has been knocked out.

| | | | | | | | P3A1 | |
|---|---|---|---|---|---|---|---|---|
| | | | | | | | hFc-vc- | 2V-hFc- |
| | P3A1 | | | | 2V hFc- | B1 hFc-vc- | PAB- | vc-PAB- |
| | hFc-va- | D3 hFc- | 2V hFc- | B1 hFc- | va- | PAB- | DMAE- | DMAE- |
| | PBD- | va-PBD- | va-PBD- | va-PAB- | PAB- | DMAE- | PNU159 | PNU159 |
| | SGD1882 | SGD1882 | SGD1882 | SG3199 | SG3199 | PNU159682 | 682 | 682 |
| Cell | IC50 | IC50 | IC50 | IC50 | IC50 | IC50 | IC50 | IC50 |
| Line | (nM) | (nM) | (nM) | (nM) | (nM) | (nM) | (nM) | (nM) |
| PA-1 | 0.065 | 0.34 | 2.5 | 0.03 | 5.9 | 0.028 | 0.0027 | 3.13 |
| PA-1 | ND | ND | ND | 0.79 | 10.5 | 1.5 | 3.4 | 4.5 |
| ROR1 | | | | | | | | |
| ko | | | | | | | | |
| Kasumi- | 0.52 | 0.25 | 6.6 | 0.06 | 4.4 | 0.8 | 5.1 | 11 |
| 2 | | | | | | | | |

The ROR1 targeting VNAR-conjugates show potent killing of both PA-1 and Kasumi-2 cancer cell-lines and show increased potency with respect to the corresponding 2V-hFc conjugates, with the IC50 values for a number of ROR1 targeting conjugates >100 fold lower than the corresponding 2V-hFc conjugate controls. Furthermore, the cell-killing effects of B1 hFc-SG3199 and B1 hFc-PNU are ROR-1 dependent—competition experiments in PA-1 and Kasumi-2 cell lines (FIG. 35b). B1 hFc inhibits cell killing by B1 hFc-SG3199 and B1 hFc-PNU in a dose-dependent manner 2VhFc did not inhibit the cell killing by these drug-conjugate molecules (FIG. 35b).

### Example 10

### ROR1 VNAR Bi-Specifics

Bispecific target combinations for ROR1 binding VNARs include, for example, HSA for half-life extension; bispecific engagement of ROR1 and serum albumin RTKs e.g. EGFR, Her3; bispecific targeting both EGFR and ROR1 or HER3 and ROR1 on the surface of cells.

The VNAR BA11, already discussed and exemplified herein, is an example of a HSA-binding VNAR. Bi-specific molecules comprising a HSA-binding VNAR (such as BA11) and another specific binding molecule are discussed.

ROR1×CD3 bispecific sequences combining N-terminal ROR1 VNARs with a C-terminal anti-CD3 scFv (clone OKT3) via 2 different length G4S linkers were expressed in CHO cells (Evitria) and purified by IMAC (HisTrap Excel, GE Healthcare) followed by SEC (Superdex 200 26/60, GE Healthcare). Similarly, biparatopic ROR1×CD3 bispecific sequences combining N-terminal biparatopic ROR1 VNARs with the C-terminal anti-CD3 scFv were also expressed in CHO (Evitria).

CD3 BiTE-like approach; examples of CD3 binding sequences for use as an ROR1 VNAR bispecificAnti CD3 scFv clone OKT3 (WO 2014028776 Zyngenia) and orientation and humanised derivatives thereof

VH-[G₄S]₃-VL
(SEQ ID NO: 100)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSDIQLTQSPAIMSA
SPGEKVTMTCRASSSVSYMNWYQQKSGTSPKRWIYDTSKVASGVPYRFS
GSGSGTSYSLTISSMEAEDAATYYCQQWSSNPLTFGAGTKLELKS

Humanised anti CD3 scFv UCHT1 (Arnett et al PNAS 2004 101(46) 16268-16273) and derivatives thereof

VL-[G₄S]₃-VH
(SEQ ID NO: 101)
MDIQMTQTTSSLSASLGDRVTISCRASQDIRNYLNWYQQKPDGTVKLLI
YYTSRLHSGVPSKFSGSGSGTDYSLTISNLEQEDIATYFCQQGNTLPWT
FAGGTKLEIKGGGGSGGGGSGGGGSEVQLQQSGPELVKPGASMKISCKA
SGYSFTGYTMNWVKQSHGKNLEWMGLINPYKGVSTYNQKFKDKATLTVD
KSSSTAYMELLSLTSEDSAVYYCARSGYYGDSDWYFDVWGQGTTLTVFS

TABLE 17
Characterisation of ROR1 × CD3 constructs

| | | MW, Da | | ROR1 |
|---|---|---|---|---|
| Name | Linker | Expected | Observed | binding |
| B1 × CD3 | G4S | 39,137.3 | 39,136.6 | Yes |
| | [G4S]3 | 39,767.9 | 39,768.2 | Yes |
| P3A1 × CD3 | G4S | 39,275.4 | 39,275.5 | Yes |
| | [G4S]3 | 39,905.9 | 39,906.8 | Yes |
| 2V × CD3 | G4S | 39,053.1 | 39,051.7 | n/d |
| | [G4S]3 | 39,683.7 | 39,682.6 | n/d |

### Biparatopic VNAR Molecules

Several biparatopic VNAR constructs were designed and cloned using a (G4S)5 linker.

Binding kinetics were determined using a SPR (as previously described) or using Biolayer interferometry (K2 Octet instrument/Pall ForteBio). For BLI experiments ROR1-hFc, ROR2-hFc fusion proteins (extracelluar domain) and HSA were immobilised in sodium acetate pH5 buffer to AR2G sensors using amine coupling. VNARs and VNAR-Fc molecules were tested at various concentrations and the Ka (M-1s-1), Kd (s-1) and KD (nM) values were determined using the Octet data analysis HT software (Pall ForteBio). 2V is a control VNAR sequence, derived from a naïve VNAR library, so is representative of this protein class but has no known target.

Binding kinetics for hROR1 binding were also performed with saturating levels of HSA (200 nM) in the baseline, association and dissociation conditions.

TABLE 18
Characterisation of biparatopic VNAR constructs

| | | | | | | | | | Cell Surface | |
|---|---|---|---|---|---|---|---|---|---|---|
| | | | | | | | | | Binding based on | |
| | | | | | | Octet or | Cell Surface Binding | | Median YL1-PE | |
| | | | | | | SPR | based on Median YL1- | | | % |
| | | | ELISA (binding at | | | hROR1 | PE at 4° C. | | MDA-MB-231 | inter- |
| | | Expression | fixed known conc.) | | | binding | A549 | A427 | (ROR1^{hi}) | nalisation |
| VNAR | Linker | System | ROR1 | ROR2 | HSA | KD (nM) | (ROR1^{hi}) | (ROR1^{low}) | 4º C. | at 37° C. |
| B1-BA11-D3 | -[G₄S]₅- | Tni (Insect) | ++++ | − | ++++ | ND | 11277 | 949 | 8079 | 38.5 |
| | | | | | + | | | | | |
| D3-BA11-B1 | -[G₄S]₅- | Tni (Insect) | +++ | − | ++++ | <0.05 | 10607 | 692 | 7142 | 48.5 |
| BA11-B1 | -[G₄S]₅- | CHO | +++ | − | ++++ | ND | 8853 | 959 | 5026 | 29.5 |
| | | | | | + | | | | | |
| B1HisMyc | NA | E.coli | ++++ | − | − | 0.5 | 8440 | 557 | 5159 | 44 |
| monomer | | | | | | | | | | |
| B1-D3 | -[G₄S]₅- | CHO | ND | | | <0.05 | 7541 | 539 | 5763 | 33.5 |
| P3A1- | -[G₄S]₅- | E.coli | ++++ | − | − | 1.0 | 6511 | 304 | 3351 | 39 |
| [G₄S]₅-B1 | | | | | | | | | | |
| B1-BA11- | -[G₄S]₅- | CHO | ND | | | ND | 6500 | 703 | 2928 | 61.5 |
| P3A1 | | | | | | | | | | |
| P3A1- | -[G₄S]₇- | E.coli | ND | | | 4.3 | 6153 | 313 | 4174 | 39 |
| [G₄S]₇-B1 | | | | | | | | | | |
| BA11-B1 | PGVQPS | CHO | ND | | | 0.78 | 6063 | 368 | 3576 | 40 |
| | PGGGGS- | | | | | | | | | |
| P3A1-BA11- | -[G₄S]₅- | Tni (Insect) | +++++ | − | ++++ | 4.46 | 5843 | 608 | 4952 | 56 |
| D3 | | | | | + | | | | | |
| P3A1- | -[G₄S]₃- | E.coli | ND | | | 2.72 | 5345 | 297 | 3908 | 30 |
| [G₄S]₃-B1 | | | | | | | | | | |
| P3A1-D3 | -[G₄S]₅- | CHO | ND | | | 1.03 | 4628 | 326 | 2687 | 0 |
| Cys | | | | | | | | | | |
| D3-BA11- | -[G₄S]₅- | Tni (Insect) | ++ | − | ++ | 1.81 | 3666 | 480 | 2534 | 57 |
| P3A1 | | | | | | | | | | |
| D3- | -[G₄S]₅- | E.coli | ++++ | − | − | 1.7 | 3168 | 302 | 1947 | 57 |
| D3HisMyc | | | | | | | | | | |
| D3-P3A1 | -[G₄S]₅- | CHO | ND | | | 1.63 | 2588 | 242 | / | / |
| Cys | | | | | | | | | | |
| P3A1- | -[G₄S]₅- | HEK293 | ++++ | − | − | 0.3 | 2335 | 257 | / | / |
| P3A1HisMyc | | | | | | | | | | |
| D3-BA11-D3 | -[G₄S]₅- | CHO | +++ | − | ++++ | 4.21 | 2126 | 259 | 1140 | 65.5 |
| | | | | | + | | | | | |
| D3-D3-BA11 | -[G₄S]₅- | CHO | ND | | | 0.76 | 2018 | 298 | 1654 | 64.5 |
| Cys | | | | | | | | | | |
| B1G1-B1G1 | -[G₄S]₅- | Tni (Insect) | ND | | | ND | 1474 | 372 | / | / |
| E9-BA11-E9 | -[G₄S]₅- | CHO | ND | | | 1.75 | 978 | 367 | / | |
| BA11-2V | -[G₄S]₅- | CHO | − | − | ++++ | ND | 446 | 246 | / | / |
| | | | | | + | | | | | |
| 2VHisMyc | NA | E.coli | − | − | − | No | 226 | 213 | / | / |
| monomer | | | | | | binding | | | | |

TABLE 19
Characterisation of additional biparatopic VNAR constructs

| | | | Octet or SPR |
|---|---|---|---|
| | | Expression | hROR1 binding |
| VNAR | Linker | System | KD (nM) |
| D3-D3-BA11 | —[G₄S]₅— | CHO | 0.764 |
| D3-P3A1 | —[G₄S]₅— | CHO | 1.63 |
| P3A1-D3 | —[G₄S]₅— | CHO | 1.03 |
| D3-B1 Cys | —[G₄S]₅— | CHO | <0.05 |

In addition, a number of bi-paratopic VNAR constructs were created using the -PGVQPSPGGGGS- linker (also termed Wobbe-G₄S) and -PGVQPAPGGGGS- linker (also termed Wobbe-G₄S-GM-) sequences. All were expressed using a QACKA HisMyc tag (SEQ ID NO: 80) and characterised and assessed as described previously.

TABLE 20
Additional data

| | | | | | | | | hROR1 | | | | | | |
|---|---|---|---|---|---|---|---|---|---|---|---|---|---|---|
| | | | | | | | | binding | | | | | Cell surface | |
| | | hROR1 binding (BLI) | | | HSA binding (BLI) | | | (+HSA) | | | | | signal at 4º C | |
| | | Ka | Kd | KD | KA | Kd | K_{D} | K_{D} | ELISA | | | | (Median YL1-PE) | |
| Construct | Linker | (M-1s1) | (s1) | (nM) | (M⁻¹) | (s⁻¹) | (nM) | (nM) | hROR1 | mROR1 | hROR2 | HSA | A549 | A427 |
| D3-P3A1 | -PGVQPSPGGGGS⁻ | 5.64E+05 | 5.33E−04 | 0.945 | — | — | No | 2.27 | ++++ | +++ | − | − | 1166 | 236 |
| | | | | | | | binding | | | | | | | |
| P3A1-D3 | -PGVQPSPGGGGS⁻ | 9.50E+04 | 2.52E−04 | 2.66 | — | — | No | 2.55 | +++ | ++ | − | − | 1470 | 232 |
| | | | | | | | binding | | | | | | | |
| B1-D3 | -PGVQPSPGGGGS⁻ | — | — | <0.05 | — | — | No | 0.252 | +++++ | ++++++ | − | − | 5573 | 1024 |
| | | | | | | | binding | | | | | | | |
| BA11- | -PGVQPSPGGGGS⁻ | 4.05E+04 | 2.90E−05 | 0.718 | 2.08E+05 | 3.86E−04 | 1.86 | <0.05 | ++++ | ++++++ | − | +++++ | 3230 | 584 |
| B1-P3A1 | | | | | | | | | | | | + | | |
| D3-P3A1- | -PGVQPSPGGGGS⁻ | 2.74E+05 | 3.63E−04 | 1.33 | 2.05E+05 | 1.31E−03 | 6.37 | 1.97 | +++++ | +++ | − | ++++ | 1204 | 264 |
| BA11 | | | | | | | | | | | | | | |
| D3-BA11- | -PGVQPSPGGGGS⁻ | 2.29E+05 | 2.48E−04 | 1.08 | 1.37E+05 | 1.22E−03 | 8.88 | 2.01 | ++++ | ++ | − | ++++ | 1807 | 312 |
| P3A1 | | | | | | | | | | | | | | |
| P3A1- | -PGVQPSPGGGGS⁻ | 1.11E+05 | 1.94E−04 | 1.74 | 1.03E+05 | 8.65E−04 | 8.37 | 3.21 | +++++ | ++++ | − | +++ | 2465 | 282 |
| BA11-D3 | | | | | | | | | | | | | | |
| P3A1-D3- | -PGVQPSPGGGGS⁻ | 1.69E+05 | 3.34E−04 | 1.98 | 2.13E+05 | 1.01E−03 | 4.73 | 2.00 | +++++ | ++++ | − | ++ | 1816 | 243 |
| BA11 | | | | | | | | | | | | | | |
| BA11-B1- | -PGVQPSPGGGGS⁻ | 1.44E+05 | <1.0E−07 | <0.05 | 5.58E+04 | 4.72E−04 | 8.47 | <0.05 | +++ | ++++ | − | + | 4069 | 387 |
| D3 | | | | | | | | | | | | | | |
| D3-BA11- | -PGVQPSPGGGGS⁻ | 1.46E+05 | 1.32E−07 | <0.05 | 2.40E+05 | 9.26E−04 | 3.86 | <0.05 | +++ | ++++ | − | +++ | 3239 | 279 |
| B1 | | | | | | | | | | | | | | |
| D3-P3A1 | -PGVQPAPGGGGS⁻ | 1.42E+05 | 3.02E−04 | 2.12 | No | No | No | 1.84 | ++++ | +++ | − | − | 1197 | 229 |
| | | | | | binding | binding | binding | | | | | | | |
| D3-P3A1- | -PGVQPAPGGGGS⁻ | 2.68E+05 | 4.14E−04 | 1.54 | 2.43E+05 | 9.24E−04 | 3.81 | 1.81 | +++ | ++++ | − | +++ | 1496 | 242 |
| BA11 | | | | | | | | | | | | | | |
| D3-BA11- | -PGVQPAPGGGGS⁻ | 2.40E+05 | 3.35E−04 | 1.39 | 1.75E+05 | 1.04E−03 | 5.94 | 1.36 | ++++ | +++ | − | ++ | 1487 | 257 |
| P3A1 | | | | | | | | | | | | | | |
| P3A1-D3 | -PGVQPAPGGGGS⁻ | 1.50E+05 | 3.61E−04 | 2.41 | — | — | No | 1.25 | +++++ | +++++ | − | − | 1805 | 231 |
| | | | | | | | binding | | | | | | | |
| P3A1- | -PGVQPAPGGGGS⁻ | 1.28E+05 | 3.23E−04 | 2.53 | 1.87E+05 | 6.92E−04 | 3.7 | 2.38 | +++++ | ++++ | − | +++ | 2531 | 261 |
| BA11-D3 | | | | | | | | | | | | | | |
| P3A1-D3- | -PGVQPAPGGGGS⁻ | 8.30E+04 | 2.37E−04 | 2.86 | 1.23E+05 | 8.37E−04 | 6.78 | 6.46 | ++++ | ++++ | − | ++ | 2043 | 247 |
| BA11 | | | | | | | | | | | | | | |
| P3A1- | -PGVQPSPGGGGS⁻ | 1.73E+05 | 4.40E−04 | 2.54 | 1.24E+05 | 6.90E−04 | 5.58 | 1.60 | +++++ | ++ | − | +++ | 1294 | 258 |
| BA11- | | | | | | | | | | | | | | |
| P3A1 | | | | | | | | | | | | | | |
| BA11- | -PGVQPSPGGGGS⁻ | 1.29E+05 | 4.47E−04 | 3.47 | 1.11E+05 | 5.66E−04 | 5.08 | 2.53 | +++ | ++++ | − | +++++ | 1005 | 269 |
| P3A1- | | | | | | | | | | | | | | |
| P3A1 | | | | | | | | | | | | | | |
| BA11-B1 | -PGVQPAPGGGGS⁻ | 5.03E+04 | 2.89E−05 | 0.58 | / | / | / | / | / | / | / | / | / | / |

The bi-paratopic VNAR constructs mentioned herein have also been successfully coupled with maleimide Alexa488 fluorophore, demonstrating that the constructs are suitable for conjugation to other moieties. This proof of concept work shows that conjugation of bi-paratopic molecules to other payloads will be possible.

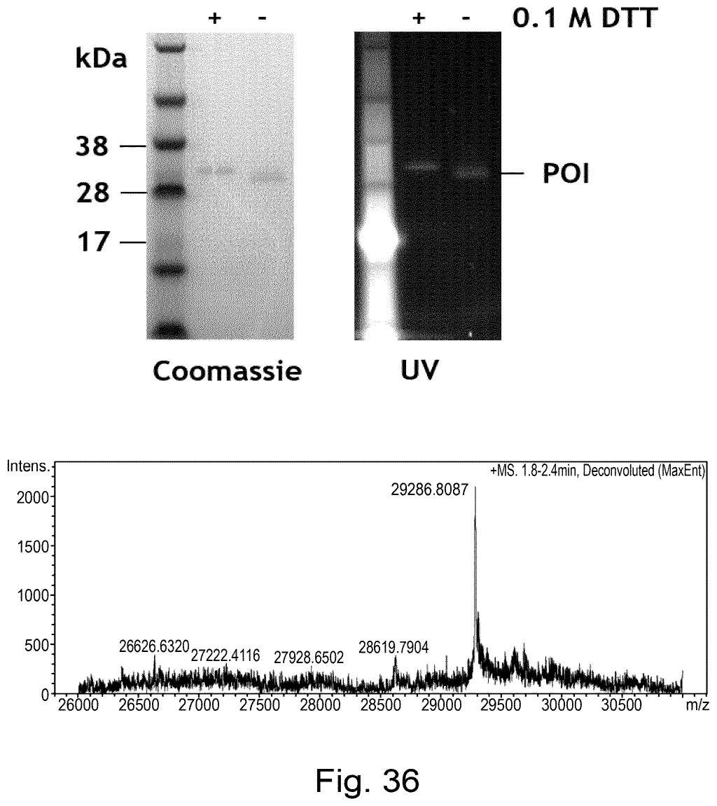

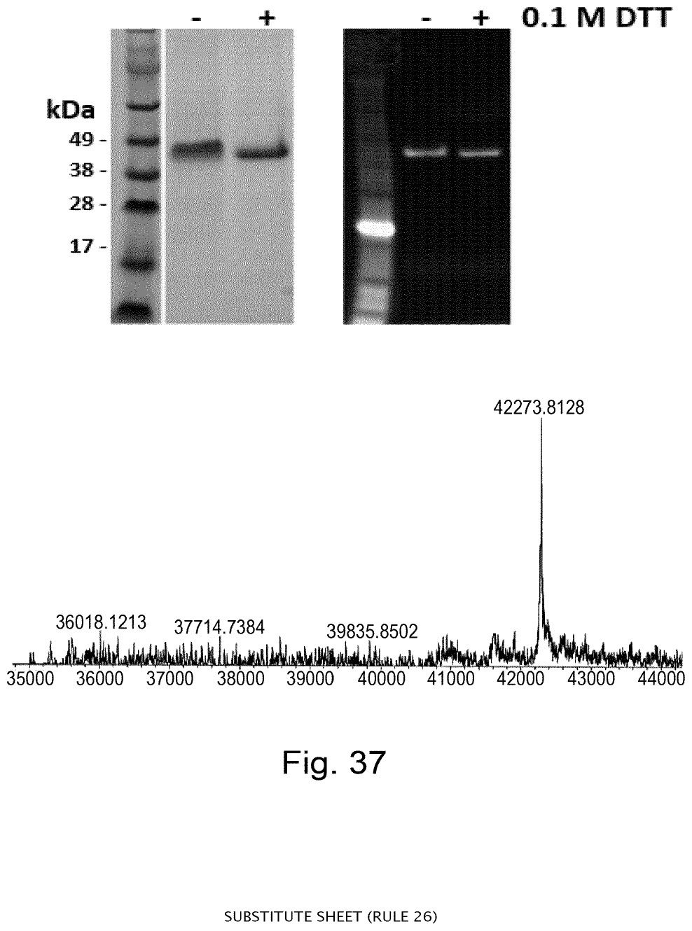

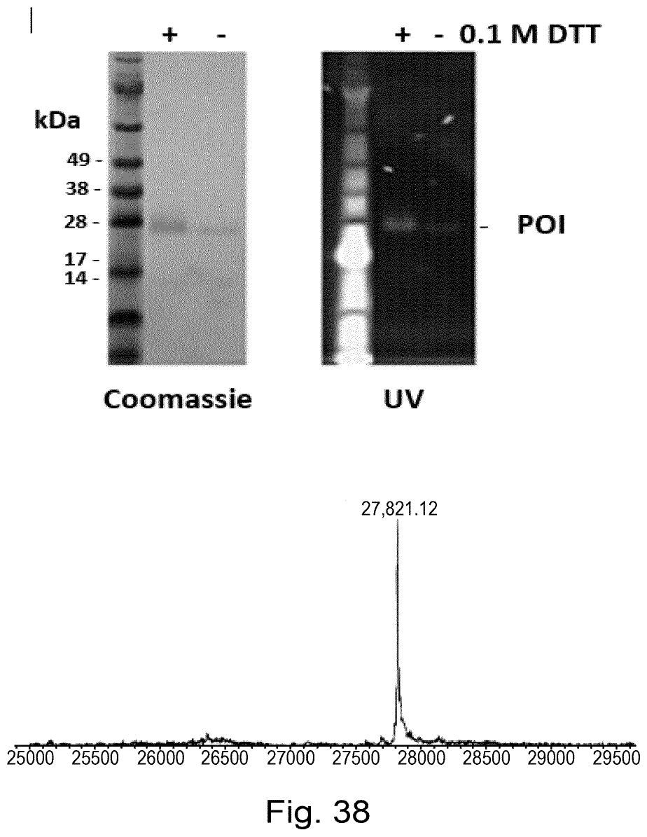

Selective labelling of VNARs with Alexa Fluor 488 C5 maleimide (Thermo Fisher Scientific, U.K.) was carried out after Ni2+ IMAC purification of the VNAR in IMAC elution buffer (typically 50 mM NaPi pH 6.9, 150 mM NaCl, 50 mM L-Arginine, 250 mM imidazole, with addition of 2 mM TCEP to remove any capping from the Cys. incorporated for conjugation). Approximately 4 molar equivalents of the Alexa Fluor 488 dye were added to the VNAR solution (typical protein concentration was 2-30 μM), and the reaction mixture was incubated in darkness for 1 hour at r.t., with gentle agitation. Reaction was monitored by LC-MS (ESI) to ensure that no unreacted VNAR remained in solution. To remove the unreacted dye, the reaction mixture was diluted with 50 mM NaPi pH 6.9, 150 mM NaCl, 50 mM L-Arginine to 50 mM imidazole concentration, and Ni2+ IMAC was carried out on ÄKTA Pure system using HisTrap Excel column (both GE Healthcare, U.K.). Protein eluted in 50 mM NaPi pH 6.9, 150 mM NaCl, 50 mM L-Arginine, 250 mM imidazole. Elution fractions containing the Alexa488 VNAR conjugate were pooled and stored at 4° C. until required. Proteins were buffer exchanged into PBS pH 7.4 or PBS pH 7.4, 50 mM L-Arg by SEC or dialysis. See FIGS. 36 to 38.

### Example 11—ROR1 CAR-T Approaches

Chimeric antigen receptors (CARs) based on the ROR1-specific antigen binding molecules described in the present application may be generated. Furthermore, engineered T cells expressing such a CAR may also be generated, which may then be used in, for example, adoptive cell therapy.

In brief, a nucleic acid construct encoding a ROR1-specific CAR may be produced. The ROR1-specific CAR may include an intracellular activation domain, a transmembrane domain, and an extracellular domain comprising the ROR1-specific antigen binding molecule described herein. The nucleic acid construct may then be incorporated into a viral vector, such as a retroviral vector (e.g., a lentiviral vector).

T cells may be isolated from a patient in need of treatment, which may then be modified to express the nucleic acid construct encoding the CAR, for example by retroviral transfection or gene-editing using approaches such as CRISPR-CAS-9.

The engineered T cells may then be re-infused into the patient in order to treat the condition, such as treatment of cancer.

1. A receptor tyrosine kinase-like orphan receptor 1 (ROR1) specific antigen binding molecule comprising an amino acid sequence represented by the formula (I):
FW1-CDR1-FW2-HV2-FW3a-HV4-FW3b-CDR3-FW4 (I)
wherein
FW1 is a framework region
CDR1 is a CDR sequence
FW2 is a framework region
HV2 is a hypervariable sequence
FW3a is a framework region
HV4 is a hypervariable sequence
FW3b is a framework region
CDR3 is a CDR sequence
FW4 is a framework region
or a functional variant thereof.

2. The ROR1-specific antigen binding molecule of claim 1, wherein the ROR1-specific antigen binding molecule does not bind to receptor tyrosine kinase-like orphan receptor 2 (ROR2).

3. The ROR1-specific antigen binding molecule of either claim 1 or claim 2, wherein the ROR1-specific antigen binding molecule binds to both human ROR1 and murine ROR1 (mROR1).

4. The ROR1-specific antigen binding molecule of any one of claims 1 to 3, wherein the ROR1-specific antigen binding molecule binds to deglycosylated ROR1.

5. The ROR1-specific antigen binding molecule of any one of claims 1 to 4, wherein the ROR1-specific antigen binding molecule does not bind to a linear peptide sequence selected from:
(SEQ ID NO: 34)
YMESLHMQGEIENQI
(SEQ ID NO: 35)
CQPWNSQYPHTHTFTALRFP
(SEQ ID NO: 36)
RSTIYGSRLRIRNLDTTDTGYFQ
(SEQ ID NO: 37)
QCVATNGKEVVSSTGVLFVKFGPPPTASPGYSDEYE

6. The ROR1-specific antigen binding molecule of any one of claims 1 to 5, wherein
FW1 is a framework region of from 20 to 28 amino acids
CDR1 is a CDR sequence selected from DTSYGLYS (SEQ ID NO: 1), GAKYGLAA (SEQ ID NO: 2), GAKYGLFA (SEQ ID NO: 3), GANYGLAA (SEQ ID NO: 4), or GANYGLAS (SEQ ID NO: 5)
FW2 is a framework region of from 6 to 14 amino acids HV2 is a hypervariable sequence selected from TTDWERMSIG (SEQ ID NO: 6), SSNQERISIS (SEQ ID NO: 7), or SSNKEQISIS (SEQ ID NO: 8)
FW3a is a framework region of from 6 to 10 amino acids
HV4 is a hypervariable sequence selected from NKRAK (SEQ ID NO: 9), NKRTM (SEQ ID NO: 10), NKGAK (SEQ ID NO: 11), or NKGTK (SEQ ID NO: 12)
FW3b is a framework region of from 17 to 24 amino acids
CDR3 is a CDR sequence selected from QSGMAISTGSGHGYNWY (SEQ ID NO: 13), QSGMAIDIGSGHGYNWY (SEQ ID NO: 14), YPWAMWGQWY (SEQ ID NO: 15), VFMPQHWHPAAHWY (SEQ ID NO: 16), REARHPWLRQWY (SEQ ID NO: 17), or YPWGAGAPWLVQWY (SEQ ID NO: 18)
FW4 is a framework region of from 7 to 14 amino acids
or a functional variant thereof with at least 45% sequence identity thereto,

7. The ROR1-specific antigen binding molecule of claim 6, wherein FW1 is selected from: ASVNQTPRTATKETGESLTINCVLT (SEQ ID NO: 19), AKVDQTPRTATKETGESLTINCVLT (SEQ ID NO: 20), TRVDQTPRTATKETGESLTINCVVT (SEQ ID NO: 21), TRVDQTPRTATKETGESLTINCVLT (SEQ ID NO: 22), ASVNQTPRTATKETGESLTINCVVT (SEQ ID NO: 23), or TRVDQSPSSLSASVGDRVTITCVLT (SEQ ID NO: 24), FW2 is selected from: TSWFRKNPG (SEQ ID NO: 25), or TYWYRKNPG (SEQ ID NO: 26); FW3a is selected from: GRYVESV (SEQ ID NO: 27), or GRYSESV (SEQ ID NO: 28), FW3b is selected from: SFSLRIKDLTVADSATYYCKA (SEQ ID NO: 29), SFTLTISSLQPEDSATYYCRA (SEQ ID NO: 30), or SFTLTISSLQPEDFATYYCKA (SEQ ID NO: 31), and FW4 is selected from: DGAGTVLTVN (SEQ ID NO: 32), or DGAGTKVEIK (SEQ ID NO: 33); or functional variants thereof with a sequence identity of at least 45%.

8. The ROR1-specific antigen binding molecule of any one of claims 1 to 7, wherein the ROR1-specific antigen binding molecule comprises an amino acid sequence selected from: ASVNQTPRTATKETGESLTINCVLTDTSYGLYSTSWFRKNPGTTDWERMSIGGRYVESVNKRAKSFSLRI KDLTVADSATYYCKAQSGMAISTGSGHGYNWYDGAGTVLTVN (SEQ ID NO: 39); AKVDQTPRTATKETGESLTINCVLTDTSYGLYSTSWFRKNPGTTDWERMSIGGRYVESVNKRAKSFSLRI KDLTVADSATYYCKAQSGMAIDIGSGHGYNWYDGAGTVLTVN (SEQ ID NO: 40); TRVDQTPRTATKETGESLTINCVVTGAKYGLAATYWYRKNPGSSNQERISISGRYVESVNKRTMSFSLRIK DLTVADSATYYCKAYPWAMWGQWYDGAGTVLTVN (SEQ ID NO: 41); TRVDQTPRTATKETGESLTINCVVTGAKYGLFATYWYRKNPGSSNQERISISGRYVESVNKRTMSFSLRIK DLTVADSATYYCKAVFMPQHWHPAAHWYDGAGTVLTVN (SEQ ID NO: 42); TRVDQTPRTATKETGESLTINCVLTDTSYGLYSTSWFRKNPGTTDWERMSIGGRYVESVNKGAKSFSLRI KDLTVADSATYYCKAREARHPWLRQWYDGAGTVLTVN (SEQ ID NO: 43); ASVNQTPRTATKETGESLTINCVVTGANYGLAATYWYRKNPGSSNQERISISGRYVESVNKRTMSFSLRIK DLTVADSATYYCKAYPWGAGAPWLVQWYDGAGTVLTVN (SEQ ID NO: 44); TRVDQSPSSLSASVGDRVTITCVLTGANYGLASTYWYRKNPGSSNKEQISISGRYSESVNKGTKSFTLTIS SLQPEDSATYYCRAYPWGAGAPWLVQWYDGAGTKVEIK (SEQ ID NO: 45); TRVDQSPSSLSASVGDRVTITCVLTGANYGLASTYWYRKNPGSSNQERISISGRYSESVNKRTMSFTLTIS SLQPEDSATYYCRAYPWGAGAPWLVQWYDGAGTKVEIK (SEQ ID NO: 46); TRVDQSPSSLSASVGDRVTITCVLTDTSYGLYSTSWFRKNPGTTDWERMSIGGRYVESVNKGAKSFTLTI SSLQPEDFATYYCKAREARHPWLRQWYDGAGTKVEIK (SEQ ID NO: 47); TRVDQSPSSLSASVGDRVTITCVLTDTSYGLYSTYWYRKNPGSSNKEQISISGRYSESVNKGTKSFTLTIS SLQPEDSATYYCRAREARHPWLRQWYDGAGTKVEIK (SEQ ID NO: 48); TRVDQSPSSLSASVGDRVTITCVLTDTSYGLYSTYWYRKNPGTTDWERMSIGGRYSESVNKGAKSFTLTI SSLQPEDSATYYCRAREARHPWLRQWYDGAGTKVEIK (SEQ ID NO: 49); or a functional variant thereof with a sequence identity of at least 45%.

9. The ROR1-specific antigen binding molecule of any one of claims 1 to 8, wherein the ROR1-specific antigen binding molecule is humanized.

10. The ROR1-specific antigen binding molecule of any one of claims 1 to 8, wherein the ROR1-specific antigen binding molecule is de-immunized.

11. The ROR1-specific antigen binding molecule of any one of claims 1 to 10, wherein the ROR1-specific antigen binding molecule is conjugated to a detectable label, dye, toxin, drug, pro-drug, radionuclide or biologically active molecule.

12. The ROR1-specific antigen binding molecule of any one of claims 1 to 11, wherein the specific antigen binding molecule selectively interacts with ROR1 protein with an affinity constant of approximately 0.01 to 50 nM, preferably 0.1 to 30 nM, even more preferably 0.1 to 10 nM.

13. The ROR1-specific antigen binding molecule of any one of claims 1 to 12, wherein the specific antigen binding molecule is capable of mediating killing of ROR1-expressing tumour cells.

14. The ROR1-specific antigen binding molecule of any one of claims 1 to 12, wherein the specific antigen binding molecule is capable of inhibiting cancer cell proliferation.

15. The ROR1-specific antigen binding molecule of any one of claims 1 to 12, wherein the specific antigen binding molecule is capable of being endocytosed upon binding to ROR1.

16. A recombinant fusion protein comprising a specific antigen binding molecule as claimed in any one of claims 1 to 15.

17. A recombinant fusion protein as claimed in claim 16, in which the specific antigen binding molecule is fused to one or more biologically active proteins.

18. A recombinant fusion protein as claimed in claim 17, wherein the specific antigen binding molecule is fused to one or more biologically active proteins via one or more linker domains.

19. The recombinant fusion protein as claimed in either claim 17 or 18, wherein at least one biologically active protein is an immunoglobin, an immunoglobulin Fc region, an immunoglobin Fab region, a single chain Fv (scFv), a diabody, a triabody, a tetrabody, a bispecific t-cell engager (BiTE), an intein, a VNAR domain, a single domain antibody (sdAb), a VH domain, or a scaffold protein.

20. The recombinant fusion protein as claimed in claim 19, wherein at least one biologically active protein is an immunoglobulin Fc region.

21. A ROR1-specific chimeric antigen receptor (CAR), comprising at least one ROR1-specific antigen binding molecule as defined in any one of claims 1 to 10, fused or conjugated to at least one transmembrane region and at least one intracellular domain.

22. A cell comprising a chimeric antigen receptor according to claim 21, which cell is preferably an engineered T-cell.

23. A nucleic acid sequence comprising a polynucleotide sequence that encodes a specific antigen binding molecule, recombinant fusion protein or chimeric antigen receptor according to any one of claims 1 to 21.

24. A vector comprising a nucleic acid sequence as claimed in claim 23, optionally further comprising one or more regulatory sequences.

25. A host cell comprising a vector as claimed in claim 24.

26. A method for preparing a specific antigen binding molecule, recombinant fusion protein or chimeric antigen receptor, comprising cultivating or maintaining a host cell comprising the polynucleotide of claim 23 under conditions such that said host cell produces the binding molecule, optionally further comprising isolating the binding molecule.

27. A pharmaceutical composition comprising the specific antigen binding molecule, recombinant fusion protein or chimeric antigen receptor of any one of claims 1 to 21.

28. The specific antigen binding molecule, recombinant fusion protein or chimeric antigen receptor of any one of claims 1 to 21, for use in therapy.

29. The specific antigen binding molecule, recombinant fusion protein or chimeric antigen receptor of any one of claims 1 to 21, for use in the treatment of cancer.

30. The specific antigen binding molecule, recombinant fusion protein or chimeric antigen receptor of claim 29, wherein the cancer is a ROR1-positive cancer type.

31. The specific antigen binding molecule, recombinant fusion protein or chimeric antigen receptor of claim 29, wherein the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer.

32. The use of a specific antigen binding molecule, recombinant fusion protein or chimeric antigen receptor of any one of claims 1 to 21 in the manufacture of a medicament for the treatment of a disease in a patient in need thereof.

33. A method of treatment of a disease in a patient in need of treatment comprising administration to said patient of a therapeutically effective dosage of a specific antigen binding molecule, recombinant fusion protein or chimeric antigen receptor of any one of claims 1 to 21 or a pharmaceutical composition of claim 27.

34. The method of claim 33, wherein the disease is cancer.

35. The method of claim 34 wherein the cancer is a ROR1-positive cancer type.

36. The method of claim 34, wherein the cancer is selected from the group comprising blood cancers such as lymphomas and leukaemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer.

37. A method of assaying for the presence of a target analyte in a sample, comprising the addition of a detectably labelled specific antigen binding molecule of any one of claims 1 to 15 or a recombinant fusion protein of claim 16 to 20 to the sample and detecting the binding of the molecule to the target analyte.

38. A method of imaging a site of disease in a subject, comprising administration of a detectably labelled specific antigen binding molecule as claimed in any one of claims 1 to 15 or a detectably labelled recombinant fusion protein of any one of claim 16 to 20 to a subject.

39. A method of diagnosis of a disease or medical condition in a subject comprising administration of a specific antigen binding molecule as claimed in any one of claims 1 to 15 or a recombinant fusion protein of claim 16 to 20.

40. An antibody, antibody fragment or antigen-binding molecule that competes for binding to ROR1 with the ROR1-specific antigen binding molecule of any one of claims 1 to 15.

41. A kit for diagnosing a subject suffering from cancer, or a pre-disposition thereto, or for providing a prognosis of the subject's condition, the kit comprising detection means for detecting the concentration of antigen present in a sample from a test subject, wherein the detection means comprises a ROR1-specific antigen binding molecule as defined in any one of claims 1 to 15, a recombinant fusion protein as defined in any one of claims 16 to 20, a CAR as defined in claim 21, or a nucleic acid as defined in claim 23, each being optionally derivatized, wherein presence of antigen in the sample suggests that the subject suffers from cancer.

42. The kit according to claim 41, wherein the antigen comprises ROR1 protein, more preferably an extracellular domain thereof.

43. The kit according to claim 41, wherein the kit is used to identify the presence or absence of ROR1-positive cells in the sample, or determine the concentration thereof in the sample.

44. The kit according to claim 41, wherein the kit comprises a positive control and/or a negative control against which the assay is compared.

45. The kit according to claim 41, wherein the kit further comprises a label which may be detected.

46. A method for diagnosing a subject suffering from cancer, or a pre-disposition thereto, or for providing a prognosis of the subject's condition, the method comprising detecting the concentration of antigen present in a sample obtained from a subject, wherein the detection is achieved using a ROR1-specific antigen binding molecule as defined in any one of claims 1 to 15, a recombinant fusion protein as defined in any one of claims 16 to 20, a CAR as define in claim 21, or a nucleic acid sequence as defined in claim 23, each being optionally derivatized, and wherein presence of antigen in the sample suggests that the subject suffers from cancer.

47. A method of killing or inhibiting the growth of a cell expressing ROR1 in vitro or in a patient, which method comprises administering to the cell a pharmaceutically effective amount or dose of (i) ROR1-specific antigen binding molecule as defined in any one of claims 1 to 15, a recombinant fusion protein as defined in any one of claims 16 to 20, a nucleic acid as defined in claim 23, or the CAR or cell according to claim 21 or 23, or (ii) of a pharmaceutical composition according to claim 27.

48. The method of claim 47, wherein the cell expressing ROR1 is a cancer cell.

49. The method according to either claim 47 or 48, wherein the ROR1 is human ROR1.

50. A specific antigen binding molecule comprising an amino acid sequence represented by the formula (II):
X-FW1-CDR1-FW2-HV2-FW3a-HV4-FW3b-CDR3-FW4-Y (II)
wherein
FW1 is a framework region
CDR1 is a CDR sequence
FW2 is a framework region
HV2 is a hypervariable sequence
FW3a is a framework region
HV4 is a hypervariable sequence
FW3b is a framework region
CDR3 is a CDR sequence
FW4 is a framework region
X and Y are optional amino acid sequences
wherein the specific antigen binding molecule is conjugated to a second moiety.

51. The specific antigen binding molecule of claim 50, wherein X or Y are individually either absent or selected from the group comprising an immunoglobulin, an immunoglobulin Fc region, an immunoglobulin Fab region, a single chain Fv (scFv), a diabody, a triabody, a tetrabody, a bispecific t-cell engager (BiTE), an intein, a VNAR domain, a single domain antibody (sdAb), a VH domain, or a scaffold protein.

52. The specific antigen binding molecule of either claim 50 or claim 51, wherein the conjugation is via a cysteine residue in the amino acid sequence of the specific antigen binding molecule.

53. The specific antigen binding molecule of either claim 50 or claim 51, wherein the conjugation is via a thiol, aminoxy or hydrazinyl moiety incorporated at the N-terminus or C-terminus of the amino acid sequence of the specific antigen binding molecule.

54. The specific antigen binding molecule of any one of claims 50 to 53, wherein the second moiety is selected from the group comprising an immunoglobulin, an immunoglobulin Fc region, an immunoglobulin Fab region, a single chain Fv (scFv), a diabody, a triabody, a tetrabody, a bispecific t-cell engager (BiTE), an intein, a VNAR domain, a single domain antibody (sdAb), a VH domain, or a scaffold protein.

55. The specific antigen binding molecule of any one of claims 50 to 53, wherein the second moiety is selected from the group comprising detectable label, dye, toxin, drug, pro-drug, radionuclide or biologically active molecule.

56. The specific antigen binding molecule according to any one of claims 50 to 53 or 55, wherein the second moiety is at least one toxin selected from the group comprising:
maytansinoids,
auristatins,
anthracyclins, preferably PNU-derived anthracyclins
amanitin derivatives, preferably α-amanitin derivatives
calicheamicins,
tubulysins
duocarmycins
radioisotopes—such as an alpha-emitting radionuclide, such as 227 Th or 225 Ac label
liposomes comprising a toxic payload,
protein toxins
taxanes
pyrrolbenzodiazepines and/or
indolinobenzodiazepine pseudodimers and/or
spliceosome inhibitors
CDK11 inhibitors
Pyridinobenzodiazepines

57. The specific antigen binding molecule according to any one of claims 50 to 56, wherein the specific antigen binding molecule is a receptor tyrosine kinase-like orphan receptor 1 (ROR1) specific antigen binding molecule.

58. The specific antigen binding molecule according to claim 57, wherein the ROR1-specific antigen binding molecule does not bind to receptor tyrosine kinase-like orphan receptor 2 (ROR2).

59. The specific antigen binding molecule according to either claim 57 or 58, wherein the ROR1-specific antigen binding molecule binds to both human ROR1 and murine ROR1 (mROR1).

60. The specific antigen binding molecule according to any one of claims 57 to 59, wherein the ROR1-specific antigen binding molecule binds to deglycosylated ROR1.

61. The specific antigen binding molecule according to any one of claims 57 to 60, wherein the ROR1-specific antigen binding molecule does not bind to a linear peptide sequence selected from:
(SEQ ID NO: 34)
YMESLHMQGEIENQI
(SEQ ID NO: 35)
CQPWNSQYPHTHTFTALRFP
(SEQ ID NO: 36)
RSTIYGSRLRIRNLDTTDTGYFQ
(SEQ ID NO: 37)
QCVATNGKEVVSSTGVLFVKFGPPPTASPGYSDEYE

62. The specific antigen binding molecule according to any one of claims 57 to 61, wherein:
FW1 is a framework region of from 20 to 28 amino acids
CDR1 is a CDR sequence selected from DTSYGLYS (SEQ ID NO: 1), GAKYGLAA (SEQ ID NO: 2), GAKYGLFA (SEQ ID NO: 3), GANYGLAA (SEQ ID NO: 4), or GANYGLAS (SEQ ID NO: 5)
FW2 is a framework region of from 6 to 14 amino acids
HV2 is a hypervariable sequence selected from TTDWERMSIG (SEQ ID NO: 6), SSNQERISIS (SEQ ID NO: 7), or SSNKEQISIS (SEQ ID NO: 8)
FW3a is a framework region of from 6 to 10 amino acids
HV4 is a hypervariable sequence selected from NKRAK (SEQ ID NO: 9), NKRTM (SEQ ID NO: 10), NKGAK (SEQ ID NO: 11), or NKGTK (SEQ ID NO: 12)
FW3b is a framework region of from 17 to 24 amino acids
CDR3 is a CDR sequence selected from QSGMAISTGSGHGYNWY (SEQ ID NO: 13), QSGMAIDIGSGHGYNWY (SEQ ID NO: 14), YPWAMWGQWY (SEQ ID NO: 15), VFMPQHWHPAAHWY (SEQ ID NO: 16), REARHPWLRQWY (SEQ ID NO: 17), or YPWGAGAPWLVQWY (SEQ ID NO: 18)
FW4 is a framework region of from 7 to 14 amino acids or a functional variant thereof with at least 45% sequence identity thereto,

63. The specific antigen binding molecule according to any one of claims 57 to 62, wherein FW1 is selected from ASVNQTPRTATKETGESLTINCVLT (SEQ ID NO: 19), AKVDQTPRTATKETGESLTINCVLT (SEQ ID NO: 20), TRVDQTPRTATKETGESLTINCVVT (SEQ ID NO: 21), TRVDQTPRTATKETGESLTINCVLT (SEQ ID NO: 22), ASVNQTPRTATKETGESLTINCVVT (SEQ ID NO: 23), or TRVDQSPSSLSASVGDRVTITCVLT (SEQ ID NO: 24), FW2 is selected from TSWFRKNPG (SEQ ID NO: 25), or TYWYRKNPG (SEQ ID NO: 26), FW3a is selected from GRYVESV (SEQ ID NO: 27), or GRYSESV (SEQ ID NO: 28), FW3b is selected from SFSLRIKDLTVADSATYYCKA (SEQ ID NO: 29), SFTLTISSLQPEDSATYYCRA (SEQ ID NO: 30), or SFTLTISSLQPEDFATYYCKA (SEQ ID NO: 31), and FW4 is selected from DGAGTVLTVN (SEQ ID NO: 32), or DGAGTKVEIK (SEQ ID NO: 33), or functional variants thereof with a sequence identity of at least 45%.

64. The specific antigen binding molecule according to any one of claims 57 to 62, wherein the ROR1-specific antigen binding molecule comprises an amino acid sequence selected from ASVNQTPRTATKETGESLTINCVLTDTSYGLYSTSWFRKNPGTTDWERMSIGGRYVESVNKRAKSFSLRI KDLTVADSATYYCKAQSGMAISTGSGHGYNWYDGAGTVLTVN (SEQ ID NO: 39); AKVDQTPRTATKETGESLTINCVLTDTSYGLYSTSWFRKNPGTTDWERMSIGGRYVESVNKRAKSFSLRI KDLTVADSATYYCKAQSGMAIDIGSGHGYNWYDGAGTVLTVN (SEQ ID NO: 40); TRVDQTPRTATKETGESLTINCVVTGAKYGLAATYWYRKNPGSSNQERISISGRYVESVNKRTMSFSLRIK DLTVADSATYYCKAYPWAMWGQWYDGAGTVLTVN (SEQ ID NO: 41); TRVDQTPRTATKETGESLTINCVVTGAKYGLFATYWYRKNPGSSNQERISISGRYVESVNKRTMSFSLRIK DLTVADSATYYCKAVFMPQHWHPAAHWYDGAGTVLTVN (SEQ ID NO: 42); TRVDQTPRTATKETGESLTINCVLTDTSYGLYSTSWFRKNPGTTDWERMSIGGRYVESVNKGAKSFSLRI KDLTVADSATYYCKAREARHPWLRQWYDGAGTVLTVN (SEQ ID NO: 43); ASVNQTPRTATKETGESLTINCVVTGANYGLAATYWYRKNPGSSNQERISISGRYVESVNKRTMSFSLRIK DLTVADSATYYCKAYPWGAGAPWLVQWYDGAGTVLTVN (SEQ ID NO: 44); TRVDQSPSSLSASVGDRVTITCVLTGANYGLASTYWYRKNPGSSNKEQISISGRYSESVNKGTKSFTLTIS SLQPEDSATYYCRAYPWGAGAPWLVQWYDGAGTKVEIK (SEQ ID NO: 45); TRVDQSPSSLSASVGDRVTITCVLTGANYGLASTYWYRKNPGSSNQERISISGRYSESVNKRTMSFTLTIS SLQPEDSATYYCRAYPWGAGAPWLVQWYDGAGTKVEIK (SEQ ID NO: 46); TRVDQSPSSLSASVGDRVTITCVLTDTSYGLYSTSWFRKNPGTTDWERMSIGGRYVESVNKGAKSFTLTI SSLQPEDFATYYCKAREARHPWLRQWYDGAGTKVEIK (SEQ ID NO: 47); TRVDQSPSSLSASVGDRVTITCVLTDTSYGLYSTYWYRKNPGSSNKEQISISGRYSESVNKGTKSFTLTIS SLQPEDSATYYCRAREARHPWLRQWYDGAGTKVEIK (SEQ ID NO: 48); TRVDQSPSSLSASVGDRVTITCVLTDTSYGLYSTYWYRKNPGTTDWERMSIGGRYSESVNKGAKSFTLTI SSLQPEDSATYYCRAREARHPWLRQWYDGAGTKVEIK (SEQ ID NO: 49); or a functional variant thereof with a sequence identity of at least 45%.

65. The specific antigen binding molecule according to any one of claims 57 to 64, wherein the ROR1-specific antigen binding molecule is humanized.

66. The specific antigen binding molecule according to any one of claims 57 to 64, wherein the ROR1-specific antigen binding molecule is de-immunized.


SEQUENCE LISTING (270 sequences)

SEQ ID NO: 1 - protein, 8 aa, synthetic construct
  source 1..8: note = Artificial sequence
DTSYGLYS

SEQ ID NO: 2 - protein, 8 aa, synthetic construct
  source 1..8: note = Artificial Sequence
GAKYGLAA

SEQ ID NO: 3 - protein, 8 aa, synthetic construct
  source 1..8: note = Artificial Sequence
GAKYGLFA

SEQ ID NO: 4 - protein, 8 aa, synthetic construct
  source 1..8: note = Artificial Sequence
GANYGLAA

SEQ ID NO: 5 - protein, 8 aa, synthetic construct
  source 1..8: note = Artificial Sequence
GANYGLAS

SEQ ID NO: 6 - protein, 10 aa, synthetic construct
  source 1..10: note = Artificial Sequence
TTDWERMSIG

SEQ ID NO: 7 - protein, 10 aa, synthetic construct
  source 1..10: note = Artificial Sequence
SSNQERISIS

SEQ ID NO: 8 - protein, 10 aa, synthetic construct
  source 1..10: note = Artificial Sequence
SSNKEQISIS

SEQ ID NO: 9 - protein, 5 aa, synthetic construct
  source 1..5: note = Artificial Sequence
NKRAK

SEQ ID NO: 10 - protein, 5 aa, synthetic construct
  source 1..5: note = Artificial Sequence
NKRTM

SEQ ID NO: 11 - protein, 5 aa, synthetic construct
  source 1..5: note = Artificial Sequence
NKGAK

SEQ ID NO: 12 - protein, 5 aa, synthetic construct
  source 1..5: note = Artificial Sequence
NKGTK

SEQ ID NO: 13 - protein, 17 aa, synthetic construct
  source 1..17: note = Artificial Sequence
QSGMAISTGSGHGYNWY

SEQ ID NO: 14 - protein, 17 aa, synthetic construct
  source 1..17: note = Artificial Sequence
QSGMAIDIGSGHGYNWY

SEQ ID NO: 15 - protein, 10 aa, synthetic construct
  source 1..10: note = Artificial Sequence
YPWAMWGQWY

SEQ ID NO: 16 - protein, 14 aa, synthetic construct
  source 1..14: note = Artificial Sequence
VFMPQHWHPAAHWY

SEQ ID NO: 17 - protein, 12 aa, synthetic construct
  source 1..12: note = Artificial Sequence
REARHPWLRQWY

SEQ ID NO: 18 - protein, 14 aa, synthetic construct
  source 1..14: note = Artificial Sequence
YPWGAGAPWLVQWY

SEQ ID NO: 19 - protein, 25 aa, synthetic construct
  source 1..25: note = Artificial Sequence
ASVNQTPRTATKETGESLTINCVLT

SEQ ID NO: 20 - protein, 25 aa, synthetic construct
  source 1..25: note = Artificial Sequence
AKVDQTPRTATKETGESLTINCVLT

SEQ ID NO: 21 - protein, 25 aa, synthetic construct
  source 1..25: note = Artificial Sequence
TRVDQTPRTATKETGESLTINCVVT

SEQ ID NO: 22 - protein, 25 aa, synthetic construct
  source 1..25: note = Artificial Sequence
TRVDQTPRTATKETGESLTINCVLT

SEQ ID NO: 23 - protein, 25 aa, synthetic construct
  source 1..25: note = Artificial Sequence
ASVNQTPRTATKETGESLTINCVVT

SEQ ID NO: 24 - protein, 25 aa, synthetic construct
  source 1..25: note = Artificial Sequence
TRVDQSPSSLSASVGDRVTITCVLT

SEQ ID NO: 25 - protein, 9 aa, synthetic construct
  source 1..9: note = Artificial Sequence
TSWFRKNPG

SEQ ID NO: 26 - protein, 9 aa, synthetic construct
  source 1..9: note = Artificial Sequence
TYWYRKNPG

SEQ ID NO: 27 - protein, 7 aa, synthetic construct
  source 1..7: note = Artificial Sequence
GRYVESV

SEQ ID NO: 28 - protein, 7 aa, synthetic construct
  source 1..7: note = Artificial Sequence
GRYSESV

SEQ ID NO: 29 - protein, 21 aa, synthetic construct
  source 1..21: note = Artificial Sequence
SFSLRIKDLTVADSATYYCKA

SEQ ID NO: 30 - protein, 21 aa, synthetic construct
  source 1..21: note = Artificial Sequence
SFTLTISSLQPEDSATYYCRA

SEQ ID NO: 31 - protein, 21 aa, synthetic construct
  source 1..21: note = Artificial Sequence
SFTLTISSLQPEDFATYYCKA

SEQ ID NO: 32 - protein, 10 aa, synthetic construct
  source 1..10: note = Artificial Sequence
DGAGTVLTVN

SEQ ID NO: 33 - protein, 10 aa, synthetic construct
  source 1..10: note = Artificial Sequence
DGAGTKVEIK

SEQ ID NO: 34 - protein, 15 aa, synthetic construct
  source 1..15: note = Artificial Sequence
YMESLHMQGEIENQI

SEQ ID NO: 35 - protein, 20 aa, synthetic construct
  source 1..20: note = Artificial Sequence
CQPWNSQYPHTHTFTALRFP

SEQ ID NO: 36 - protein, 23 aa, synthetic construct
  source 1..23: note = Artificial Sequence
RSTIYGSRLRIRNLDTTDTGYFQ

SEQ ID NO: 37 - protein, 36 aa, synthetic construct
  source 1..36: note = Artificial Sequence
QCVATNGKEVVSSTGVLFVKFGPPPTASPGYSDEYE

SEQ ID NO: 38 - protein, 22 aa, synthetic construct
  source 1..22: note = Artificial Sequence
RSTIYGSRLRINLDTTDTGYFQ

SEQ ID NO: 39 - protein, 112 aa, synthetic construct
  source 1..112: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVN

SEQ ID NO: 40 - protein, 112 aa, synthetic construct
  source 1..112: note = Artificial Sequence
AKVDQTPRTATKETGESLTINCVLTDTSYGLYSTSWFRKNPGTTDWERMSIGGRYVESVNKRAKSFSLRIKDLTVADSATYYCKAQSGMAIDIGSGHGYNWYDGAGTVLTVN

SEQ ID NO: 41 - protein, 105 aa, synthetic construct
  source 1..105: note = Artificial Sequence
TRVDQTPRTATKETGESLTINCVVTGAKYGLAATYWYRKNPGSSNQERISISGRYVESVNKRTMSFSLRIKDLTVADSATYYCKAYPWAMWGQWYDGAGTVLTVN

SEQ ID NO: 42 - protein, 109 aa, synthetic construct
  source 1..109: note = Artificial Sequence
TRVDQTPRTATKETGESLTINCVVTGAKYGLFATYWYRKNPGSSNQERISISGRYVESVNKRTMSFSLRIKDLTVADSATYYCKAVFMPQHWHPAAHWYDGAGTVLTVN

SEQ ID NO: 43 - protein, 107 aa, synthetic construct
  source 1..107: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVN

SEQ ID NO: 44 - protein, 109 aa, synthetic construct
  source 1..109: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVN

SEQ ID NO: 45 - protein, 109 aa, synthetic construct
  source 1..109: note = Artificial Sequence
TRVDQSPSSLSASVGDRVTITCVLTGANYGLASTYWYRKNPGSSNKEQISISGRYSESVNKGTKSFTLTISSLQPEDSATYYCRAYPWGAGAPWLVQWYDGAGTKVEIK

SEQ ID NO: 46 - protein, 109 aa, synthetic construct
  source 1..109: note = Artificial Sequence
TRVDQSPSSLSASVGDRVTITCVLTGANYGLASTYWYRKNPGSSNQERISISGRYSESVNKRTMSFTLTISSLQPEDSATYYCRAYPWGAGAPWLVQWYDGAGTKVEIK

SEQ ID NO: 47 - protein, 107 aa, synthetic construct
  source 1..107: note = Artificial Sequence
TRVDQSPSSLSASVGDRVTITCVLTDTSYGLYSTSWFRKNPGTTDWERMSIGGRYVESVNKGAKSFTLTISSLQPEDFATYYCKAREARHPWLRQWYDGAGTKVEIK

SEQ ID NO: 48 - protein, 107 aa, synthetic construct
  source 1..107: note = Artificial Sequence
TRVDQSPSSLSASVGDRVTITCVLTDTSYGLYSTYWYRKNPGSSNKEQISISGRYSESVNKGTKSFTLTISSLQPEDSATYYCRAREARHPWLRQWYDGAGTKVEIK

SEQ ID NO: 49 - protein, 107 aa, synthetic construct
  source 1..107: note = Artificial Sequence
TRVDQSPSSLSASVGDRVTITCVLTDTSYGLYSTYWYRKNPGTTDWERMSIGGRYSESVNKGAKSFTLTISSLQPEDSATYYCRAREARHPWLRQWYDGAGTKVEIK

SEQ ID NO: 50 - protein, 112 aa, synthetic construct
  source 1..112: note = Artificial Sequence
ASVNQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIK

SEQ ID NO: 51 - protein, 112 aa, synthetic construct
  source 1..112: note = Artificial Sequence
TRVDQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIK

SEQ ID NO: 52 - protein, 112 aa, synthetic construct
  source 1..112: note = Artificial Sequence
ASVNQSPSSASASVGDRLTITCVLTDTSYGLYSTSWFRKNPGTTDWERMSIGGRYSESVNKGAKSFTLTISSLQPEDSATYYCKAQSGMAISTGSGHGYNWYDGAGTKLEVK

SEQ ID NO: 53 - protein, 109 aa, synthetic construct
  source 1..109: note = Artificial Sequence
ASVDQSPSSLSASVGDRVTITCVVTGANYGLAATYWYRKNPGSSNQERISISGRYSESVNKRTMSFTLTISSLQPEDSATYYCKAYPWGAGAPWLVQWYDGAGTKVEIK

SEQ ID NO: 54 - protein, 109 aa, synthetic construct
  source 1..109: note = Artificial Sequence
ASVDQSPSSASASVGDRLTITCVVTGANYGLAATYWYRKNPGSSNQERISISGRYSESVNKRTMSFTLTISSLQPEDSATYYCKAYPWGAGAPWLVQWYDGAGTKLEVK

SEQ ID NO: 55 - protein, 112 aa, synthetic construct
  source 1..112: note = Artificial Sequence
ASVNQSPSSLSASVGDRVTITCVLTDTSYGLYSTSWFRKNPGTTDWERMSIGGRYVESVNKRAKSFSLRIKDLTVADSATYYCKAQSGMAISTGSGHGYNWYDGAGTKVEIK

SEQ ID NO: 56 - protein, 112 aa, synthetic construct
  source 1..112: note = Artificial Sequence
ASVNQSPSSLSASVGDRVTITCVLTDTSYGLYSTSWFRKNPGTTDWERMSIGGRYVESVNKRAKSFTLTISSLQPEDFATYYCKAQSGMAISTGSGHGYNWYDGAGTKVEIK

SEQ ID NO: 57 - protein, 112 aa, synthetic construct
  source 1..112: note = Artificial Sequence
ASVNQSPSSLSASVGDRVTITCVLTDTSYGLYSTSWFRKNPGTTDWERMSIGGRFSGSGSKRAKSFTLTISSLQPEDFATYYCKAQSGMAISTGSGHGYNWYDGAGTKVEIK

SEQ ID NO: 58 - protein, 112 aa, synthetic construct
  source 1..112: note = Artificial Sequence
ASVNQSPSSLSASVGDRVTITCVLTDTSYGLYSTSWYQQKPGTTDWERMSIGGRYVESVNKRAKSFTLTISSLQPEDFATYYCKAQSGMAISTGSGHGYNWYDGAGTKVEIK

SEQ ID NO: 59 - protein, 112 aa, synthetic construct
  source 1..112: note = Artificial Sequence
ASVNQSPSSLSASVGDRVTITCVLTDTSYGLYSTSWYQQKPGTTDWERMSIGGRFSGSGSKRAKSFTLTISSLQPEDFATYYCKAQSGMAISTGSGHGYNWYDGAGTKVEIK

SEQ ID NO: 60 - protein, 15 aa, synthetic construct
  source 1..15: note = Artificial Sequence
GGGGSGGGGSGGGGS

SEQ ID NO: 61 - protein, 25 aa, synthetic construct
  source 1..25: note = Artificial Sequence
GGGGSGGGGSGGGGSGGGGSGGGGS

SEQ ID NO: 62 - protein, 7 aa, synthetic construct
  source 1..7: note = Artificial Sequence
PGVQPSP

SEQ ID NO: 63 - protein, 12 aa, synthetic construct
  source 1..12: note = Artificial Sequence
PGVQPSPGGGGS

SEQ ID NO: 64 - protein, 12 aa, synthetic construct
  source 1..12: note = Artificial Sequence
PGVQPAPGGGGS

SEQ ID NO: 65 - protein, 107 aa, synthetic construct
  source 1..107: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVN

SEQ ID NO: 66 - protein, 103 aa, synthetic construct
  source 1..103: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIK

SEQ ID NO: 67 - protein, 25 aa, synthetic construct
  source 1..25: note = Artificial Sequence
QACGAHHHHHHGAEFEQKLISEEDL

SEQ ID NO: 68 - protein, 10 aa, synthetic construct
  source 1..10: note = Artificial Sequence
EQKLISEEDL

SEQ ID NO: 69 - protein, 25 aa, synthetic construct
  source 1..25: note = Artificial Sequence
QASGAHHHHHHGAEFEQKLISEEDL

SEQ ID NO: 70 - protein, 25 aa, synthetic construct
  source 1..25: note = Artificial Sequence
QACKAHHHHHHGAEFEQKLISEEDL

SEQ ID NO: 71 - protein, 23 aa, synthetic construct
  source 1..23: note = Artificial Sequence
AAAHHHHHHGAEFEQKLISEEDL

SEQ ID NO: 72 - protein, 23 aa, synthetic construct
  source 1..23: note = Artificial Sequence
ACAHHHHHHGAEFEQKLISEEDL

SEQ ID NO: 73 - protein, 11 aa, synthetic construct
  source 1..11: note = Artificial Sequence
QASGAHHHHHH

SEQ ID NO: 74 - protein, 11 aa, synthetic construct
  source 1..11: note = Artificial Sequence
QACGAHHHHHH

SEQ ID NO: 75 - protein, 11 aa, synthetic construct
  source 1..11: note = Artificial Sequence
QACKAHHHHHH

SEQ ID NO: 76 - protein, 9 aa, synthetic construct
  source 1..9: note = Artificial Sequence
AAAHHHHHH

SEQ ID NO: 77 - protein, 9 aa, synthetic construct
  source 1..9: note = Artificial Sequence
ACAHHHHHH

SEQ ID NO: 78 - protein, 5 aa, synthetic construct
  source 1..5: note = Artificial Sequence
QASGA

SEQ ID NO: 79 - protein, 5 aa, synthetic construct
  source 1..5: note = Artificial Sequence
QACGA

SEQ ID NO: 80 - protein, 5 aa, synthetic construct
  source 1..5: note = Artificial Sequence
QACKA

SEQ ID NO: 81 - skipped

SEQ ID NO: 82 - protein, 5 aa, synthetic construct
  source 1..5: note = Artificial Sequence
SAPSA

SEQ ID NO: 83 - protein, 361 aa, synthetic construct
  source 1..361: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 84 - protein, 371 aa, synthetic construct
  source 1..371: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 85 - protein, 359 aa, synthetic construct
  source 1..359: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 86 - protein, 369 aa, synthetic construct
  source 1..369: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 87 - protein, 491 aa, synthetic construct
  source 1..491: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 88 - protein, 501 aa, synthetic construct
  source 1..501: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 89 - protein, 480 aa, synthetic construct
  source 1..480: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 90 - protein, 490 aa, synthetic construct
  source 1..490: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 91 - protein, 483 aa, synthetic construct
  source 1..483: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 92 - protein, 493 aa, synthetic construct
  source 1..493: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 93 - protein, 496 aa, synthetic construct
  source 1..496: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 94 - protein, 506 aa, synthetic construct
  source 1..506: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 95 - protein, 233 aa, synthetic construct
  source 1..233: note = Artificial Sequence
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 96 - protein, 232 aa, synthetic construct
  source 1..232: note = Artificial Sequence
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 26 aa, synthetic construct
  source 1..26: note = Artificial Sequence
QASGAHHHHHHGAEFEQKLISEEDLG

SEQ ID NO: 98 - protein, 446 aa, synthetic construct
  source 1..446: note = Artificial Sequence
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 214 aa, synthetic construct
  source 1..214: note = Artificial Sequence
DIVMTQTPLSLPVTPGEPASISCRASKSISKYLAWYQQKPGQAPRLLIYSGSTLQSGIPPRFSGSGTGTDFTLTINNIESEDAAYYFCQQHDESPYTFGEGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 241 aa, synthetic construct
  source 1..241: note = Artificial Sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 101 - protein, 245 aa, synthetic construct
  source 1..245: note = Artificial Sequence
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 102 - protein, 105 aa, synthetic construct; identical to SEQ ID NO: 41
  source 1..105: note = Artificial Sequence

SEQ ID NO: 103 - protein, 109 aa, synthetic construct; identical to SEQ ID NO: 42
  source 1..109: note = Artificial Sequence

SEQ ID NO: 104 - protein, 130 aa, synthetic construct
  source 1..130: note = Artificial Sequence
TRVDQTPRTATKETGESLTINCVVTGAKYGLAATYWYRKNPGSSNQERISISGRYVESVNKRTMSFSLRIKDLTVADSATYYCKAYPWAMWGQWYDGAGTVLTVNQASGAHHHHHHGAEFEQKLISEEDL

SEQ ID NO: 105 - protein, 134 aa, synthetic construct
  source 1..134: note = Artificial Sequence
TRVDQTPRTATKETGESLTINCVVTGAKYGLFATYWYRKNPGSSNQERISISGRYVESVNKRTMSFSLRIKDLTVADSATYYCKAVFMPQHWHPAAHWYDGAGTVLTVNQASGAHHHHHHGAEFEQKLISEEDL

SEQ ID NO: 106 - protein, 132 aa, synthetic construct
  source 1..132: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHGAEFEQKLISEEDL

SEQ ID NO: 107 - protein, 134 aa, synthetic construct
  source 1..134: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 108 - protein, 137 aa, synthetic construct
  source 1..137: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 109 - protein, 137 aa, synthetic construct
  source 1..137: note = Artificial Sequence
AKVDQTPRTATKETGESLTINCVLTDTSYGLYSTSWFRKNPGTTDWERMSIGGRYVESVNKRAKSFSLRIKDLTVADSATYYCKAQSGMAIDIGSGHGYNWYDGAGTVLTVNQASGAHHHHHHGAEFEQKLISEEDL

SEQ ID NO: 110 - protein, 248 aa, synthetic construct
  source 1..248: note = Artificial Sequence
GGGGSGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 111 - protein, 247 aa, synthetic construct
  source 1..247: note = Artificial Sequence
GGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 247 aa, synthetic construct
  source 1..247: note = Artificial Sequence
GGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 247 aa, synthetic construct
  source 1..247: note = Artificial Sequence
GGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 114 - protein, 252 aa, synthetic construct
  source 1..252: note = Artificial Sequence
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGS

SEQ ID NO: 115 - protein, 253 aa, synthetic construct
  source 1..253: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 116 - protein, 253 aa, synthetic construct
  source 1..253: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACKAHHHHHHGAEFEQKLISEEDL

SEQ ID NO: 117 - protein, 256 aa, synthetic construct
  source 1..256: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 118 - protein, 256 aa, synthetic construct
  source 1..256: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 119 - protein, 258 aa, synthetic construct
  source 1..258: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNPGVQPSPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 120 - protein, 368 aa, synthetic construct
  source 1..368: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 121 - protein, 368 aa, synthetic construct
  source 1..368: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 122 - protein, 368 aa, synthetic construct
  source 1..368: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACKAHHHHHHGAEFEQKLISEEDL

SEQ ID NO: 123 - protein, 371 aa, synthetic construct
  source 1..371: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQACKAHHHHHHGAEFEQKLISEEDL

SEQ ID NO: 124 - protein, 371 aa, synthetic construct
  source 1..371: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 125 - protein, 371 aa, synthetic construct
  source 1..371: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 126 - protein, 371 aa, synthetic construct
  source 1..371: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQACKAHHHHHHGAEFEQKLISEEDL

SEQ ID NO: 127 - protein, 373 aa, synthetic construct
  source 1..373: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 128 - protein, 373 aa, synthetic construct
  source 1..373: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNPGVQPSPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 129 - protein, 373 aa, synthetic construct
  source 1..373: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACKAHHHHHHGAEFEQKLISEEDL

SEQ ID NO: 130 - protein, 256 aa, synthetic construct
  source 1..256: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPAPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 131 - protein, 371 aa, synthetic construct
  source 1..371: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPAPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQACKAHHHHHHGAEFEQKLISEEDL

SEQ ID NO: 132 - protein, 371 aa, synthetic construct
  source 1..371: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPAPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 133 - protein, 256 aa, synthetic construct
  source 1..256: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 134 - protein, 371 aa, synthetic construct
  source 1..371: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPAPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACKAHHHHHHGAEFEQKLISEEDL

SEQ ID NO: 135 - protein, 371 aa, synthetic construct
  source 1..371: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPAPGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNPGVQPAPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQACKAHHHHHHGAEFEQKLISEEDL

SEQ ID NO: 136 - protein, 366 aa, synthetic construct
  source 1..366: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 137 - protein, 366 aa, synthetic construct
  source 1..366: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNPGVQPSPGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACKAHHHHHHGAEFEQKLISEEDL

SEQ ID NO: 138 - protein, 251 aa, synthetic construct
  source 1..251: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNPGVQPSPGGGGSTRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNQACKAHHHHHHGAEFEQKLISEEDL

SEQ ID NO: 139 - protein, 366 aa, synthetic construct
  source 1..366: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNPGVQPSPGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGSTRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNQACKAHHHHHHGAEFEQKLISEEDL

SEQ ID NO: 140 - protein, 250 aa, synthetic construct
  source 1..250: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 141 - protein, 250 aa, synthetic construct
  source 1..250: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPAPGGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 142 - protein, 250 aa, synthetic construct
  source 1..250: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPSPGGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 143 - protein, 250 aa, synthetic construct
  source 1..250: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKPGVQPAPGGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 144 - protein, 271 aa, synthetic construct
  source 1..271: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 145 - protein, 269 aa, synthetic construct
  source 1..269: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHGAEFEQKLISEEDL

SEQ ID NO: 146 - protein, 269 aa, synthetic construct
  source 1..269: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 147 - protein, 271 aa, synthetic construct
  source 1..271: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 148 - protein, 271 aa, synthetic construct
  source 1..271: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACGAHHHHHHGAEFEQKLISEEDL

SEQ ID NO: 149 - protein, 269 aa, synthetic construct
  source 1..269: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQACGAHHHHHHGAEFEQKLISEEDL

SEQ ID NO: 150 - protein, 269 aa, synthetic construct
  source 1..269: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACGAHHHHHHGAEFEQKLISEEDL

SEQ ID NO: 151 - protein, 394 aa, synthetic construct
  source 1..394: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHGAEFEQKLISEEDL

SEQ ID NO: 152 - protein, 399 aa, synthetic construct
  source 1..399: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 153 - protein, 399 aa, synthetic construct
  source 1..399: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 154 - protein, 399 aa, synthetic construct
  source 1..399: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 155 - protein, 397 aa, synthetic construct
  source 1..397: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHGAEFEQKLISEEDL

SEQ ID NO: 156 - protein, 397 aa, synthetic construct
  source 1..397: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACGAHHHHHHGAEFEQKLISEEDL

SEQ ID NO: 157 - protein, 397 aa, synthetic construct
  source 1..397: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 158 - protein, 266 aa, synthetic construct
  source 1..266: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 159 - protein, 255 aa, synthetic construct
  source 1..255: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHH

SEQ ID NO: 160 - protein, 246 aa, synthetic construct
  source 1..246: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNSAPSA

SEQ ID NO: 161 - protein, 260 aa, synthetic construct
  source 1..260: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQACGAHHHHHHGAEFEQKLISEEDL

SEQ ID NO: 162 - protein, 260 aa, synthetic construct
  source 1..260: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQASGAHHHHHHGAEFEQKLISEEDL

SEQ ID NO: 163 - protein, 260 aa, synthetic construct
  source 1..260: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNQASGAHHHHHHGAEFEQKLISEEDL

SEQ ID NO: 164 - protein, 402 aa, synthetic construct
  source 1..402: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQASGAHHHHHHGAEFEQKLISEEDL

SEQ ID NO: 165 - protein, 402 aa, synthetic construct
  source 1..402: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKQACGAHHHHHHGAEFEQKLISEEDL

SEQ ID NO: 166 - protein, 402 aa, synthetic construct
  source 1..402: note = Artificial Sequence
AKVDQTPRTATKETGESLTINCVLTDTSYGLYSTSWFRKNPGTTDWERMSIGGRYVESVNKRAKSFSLRIKDLTVADSATYYCKAQSGMAIDIGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSAKVDQTPRTATKETGESLTINCVLTDTSYGLYSTSWFRKNPGTTDWERMSIGGRYVESVNKRAKSFSLRIKDLTVADSATYYCKAQSGMAIDIGSGHGYNWYDGAGTVLTVNQASGAHHHHHHGAEFEQKLISEEDL

SEQ ID NO: 167 - protein, 402 aa, synthetic construct
  source 1..402: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 168 - protein, 392 aa, synthetic construct
  source 1..392: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHGAEFEQKLISEEDL

SEQ ID NO: 169 - protein, 262 aa, synthetic construct
  source 1..262: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 170 - protein, 249 aa, synthetic construct
  source 1..249: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNSPSTPPTPSPTPPATRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKACAHHHHHHGAEFEQKLISEEDL

SEQ ID NO: 171 - protein, 251 aa, synthetic construct
  source 1..251: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKSPSTPPTPSPTPPAASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 172 - protein, 283 aa, synthetic construct
  source 1..283: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKAEAAAKEAAAKEAAAKEAAAKALEAEAAAKEAAAKEAAAKEAAAKAASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 173 - protein, 264 aa, synthetic construct
  source 1..264: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKSPSTPPTPSPTPPSPSTPPTPSPTPPAASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 174 - protein, 249 aa, synthetic construct; identical to SEQ ID NO: 170
  source 1..249: note = Artificial Sequence

SEQ ID NO: 175 - protein, 251 aa, synthetic construct; identical to SEQ ID NO: 171
  source 1..251: note = Artificial Sequence

SEQ ID NO: 176 - protein, 283 aa, synthetic construct; identical to SEQ ID NO: 172
  source 1..283: note = Artificial Sequence

SEQ ID NO: 177 - protein, 264 aa, synthetic construct; identical to SEQ ID NO: 173
  source 1..264: note = Artificial Sequence

SEQ ID NO: 178 - protein, 256 aa, synthetic construct
  source 1..256: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 179 - protein, 276 aa, synthetic construct
  source 1..276: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 180 - protein, 252 aa, synthetic construct
  source 1..252: note = Artificial Sequence
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIKGGGGSGGGGSGGGGSASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHHGAEFEQKLISEEDL

SEQ ID NO: 181 - protein, 107 aa, synthetic construct; identical to SEQ ID NO: 65
  source 1..107: note = Artificial Sequence

SEQ ID NO: 182 - protein, 134 aa, synthetic construct; identical to SEQ ID NO: 107
  source 1..134: note = Artificial Sequence

SEQ ID NO: 183 - protein, 120 aa, synthetic construct
  source 1..120: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHH

SEQ ID NO: 184 - protein, 114 aa, synthetic construct
  source 1..114: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGA

SEQ ID NO: 185 - protein, 120 aa, synthetic construct
  source 1..120: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACGAHHHHHH

SEQ ID NO: 186 - protein, 120 aa, synthetic construct
  source 1..120: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQACKAHHHHHH

SEQ ID NO: 187 - protein, 114 aa, synthetic construct
  source 1..114: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNSAPSA

SEQ ID NO: 188 - protein, 123 aa, synthetic construct
  source 1..123: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNSPSTPPTPSPTPPA

SEQ ID NO: 189 - protein, 134 aa, synthetic construct
  source 1..134: note = Artificial Sequence
TRVDQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNQASGAHHHHHHGAEFEQKLISEEDL

SEQ ID NO: 190 - protein, 132 aa, synthetic construct; identical to SEQ ID NO: 106
  source 1..132: note = Artificial Sequence

SEQ ID NO: 191 - protein, 119 aa, synthetic construct
  source 1..119: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHH

SEQ ID NO: 192 - protein, 107 aa, synthetic construct; identical to SEQ ID NO: 43
  source 1..107: note = Artificial Sequence

SEQ ID NO: 193 - protein, 137 aa, synthetic construct; identical to SEQ ID NO: 108
  source 1..137: note = Artificial Sequence

SEQ ID NO: 194 - protein, 137 aa, synthetic construct; identical to SEQ ID NO: 109
  source 1..137: note = Artificial Sequence

SEQ ID NO: 195 - protein, 130 aa, synthetic construct; identical to SEQ ID NO: 104
  source 1..130: note = Artificial Sequence

SEQ ID NO: 196 - protein, 134 aa, synthetic construct; identical to SEQ ID NO: 105
  source 1..134: note = Artificial Sequence

SEQ ID NO: 197 - protein, 103 aa, synthetic construct; identical to SEQ ID NO: 66
  source 1..103: note = Artificial Sequence

SEQ ID NO: 198 - protein, 134 aa, synthetic construct
  source 1..134: note = Artificial Sequence
TRVDQSPSSLSASVGDRVTITCVLTGANYGLASTYWYRKNPGSSNKEQISISGRYSESVNKGTKSFTLTISSLQPEDSATYYCRAYPWGAGAPWLVQWYDGAGTKVEIKQASGAHHHHHHGAEFEQKLISEEDL

SEQ ID NO: 199 - protein, 134 aa, synthetic construct
  source 1..134: note = Artificial Sequence
TRVDQSPSSLSASVGDRVTITCVLTGANYGLASTYWYRKNPGSSNQERISISGRYSESVNKRTMSFTLTISSLQPEDSATYYCRAYPWGAGAPWLVQWYDGAGTKVEIKQASGAHHHHHHGAEFEQKLISEEDL

SEQ ID NO: 200 - protein, 134 aa, synthetic construct
  source 1..134: note = Artificial Sequence
ASVNQSPSSLSASVGDRVTITCVVTGANYGLAATYWYRKNPGSSNQERISISGRYVESVNKRTMSFTLTISSLQPEDFATYYCKAYPWGAGAPWLVQWYDGAGTKVEIKQASGAHHHHHHGAEFEQKLISEEDL

SEQ ID NO: 201 - protein, 134 aa, synthetic construct
  source 1..134: note = Artificial Sequence
ASVDQSPSSLSASVGDRVTITCVVTGANYGLAATYWYRKNPGSSNQERISISGRYSESVNKRTMSFTLTISSLQPEDSATYYCKAYPWGAGAPWLVQWYDGAGTKVEIKQASGAHHHHHHGAEFEQKLISEEDL

SEQ ID NO: 202 - protein, 134 aa, synthetic construct
  source 1..134: note = Artificial Sequence
ASVDQSPSSASASVGDRLTITCVVTGANYGLAATYWYRKNPGSSNQERISISGRYSESVNKRTMSFTLTISSLQPEDSATYYCKAYPWGAGAPWLVQWYDGAGTKLEVKQASGAHHHHHHGAEFEQKLISEEDL

SEQ ID NO: 203 - protein, 137 aa, synthetic construct
  source 1..137: note = Artificial Sequence
ASVNQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIKQASGAHHHHHHGAEFEQKLISEEDL

SEQ ID NO: 204 - protein, 137 aa, synthetic construct
  source 1..137: note = Artificial Sequence
TRVDQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIKQASGAHHHHHHGAEFEQKLISEEDL

SEQ ID NO: 205 - protein, 137 aa, synthetic construct
  source 1..137: note = Artificial Sequence
ASVNQSPSSASASVGDRLTITCVLTDTSYGLYSTSWFRKNPGTTDWERMSIGGRYSESVNKGAKSFTLTISSLQPEDSATYYCKAQSGMAISTGSGHGYNWYDGAGTKLEVKQASGAHHHHHHGAEFEQKLISEEDL

SEQ ID NO: 206 - protein, 137 aa, synthetic construct
  source 1..137: note = Artificial Sequence
ASVNQSPSSLSASVGDRVTITCVLTDTSYGLYSTSWFRKNPGTTDWERMSIGGRYVESVNKRAKSFSLRIKDLTVADSATYYCKAQSGMAISTGSGHGYNWYDGAGTKVEIKQASGAHHHHHHGAEFEQKLISEEDL

SEQ ID NO: 207 - protein, 137 aa, synthetic construct
  source 1..137: note = Artificial Sequence
ASVNQSPSSLSASVGDRVTITCVLTDTSYGLYSTSWFRKNPGTTDWERMSIGGRYVESVNKRAKSFTLTISSLQPEDFATYYCKAQSGMAISTGSGHGYNWYDGAGTKVEIKQASGAHHHHHHGAEFEQKLISEEDL

SEQ ID NO: 208 - protein, 137 aa, synthetic construct
  source 1..137: note = Artificial Sequence
ASVNQSPSSLSASVGDRVTITCVLTDTSYGLYSTSWFRKNPGTTDWERMSIGGRFSGSGSKRAKSFTLTISSLQPEDFATYYCKAQSGMAISTGSGHGYNWYDGAGTKVEIKQASGAHHHHHHGAEFEQKLISEEDL

SEQ ID NO: 209 - protein, 137 aa, synthetic construct
  source 1..137: note = Artificial Sequence
ASVNQSPSSLSASVGDRVTITCVLTDTSYGLYSTSWYQQKPGTTDWERMSIGGRYVESVNKRAKSFTLTISSLQPEDFATYYCKAQSGMAISTGSGHGYNWYDGAGTKVEIKQASGAHHHHHHGAEFEQKLISEEDL

SEQ ID NO: 210 - protein, 137 aa, synthetic construct
  source 1..137: note = Artificial Sequence
ASVNQSPSSLSASVGDRVTITCVLTDTSYGLYSTSWYQQKPGTTDWERMSIGGRFSGSGSKRAKSFTLTISSLQPEDFATYYCKAQSGMAISTGSGHGYNWYDGAGTKVEIKQASGAHHHHHHGAEFEQKLISEEDL

SEQ ID NO: 211 - protein, 264 aa, synthetic construct
  source 1..264: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHHGAEFEQKLISEEDL

SEQ ID NO: 212 - protein, 250 aa, synthetic construct
  source 1..250: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNQASGAHHHHHH

SEQ ID NO: 213 - protein, 244 aa, synthetic construct
  source 1..244: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNSAPSA

SEQ ID NO: 214 - protein, 274 aa, synthetic construct
  source 1..274: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHHGAEFEQKLISEEDL

SEQ ID NO: 215 - protein, 260 aa, synthetic construct
  source 1..260: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQASGAHHHHHH

SEQ ID NO: 216 - protein, 268 aa, synthetic construct
  source 1..268: note = Artificial Sequence
TRVDQTPRTATKETGESLTINCVVTGAKYGLFATYWYRKNPGSSNQERISISGRYVESVNKRTMSFSLRIKDLTVADSATYYCKAVFMPQHWHPAAHWYDGAGTVLTVNGGGGSGGGGSGGGGSGGGGSGGGGSTRVDQTPRTATKETGESLTINCVVTGAKYGLFATYWYRKNPGSSNQERISISGRYVESVNKRTMSFSLRIKDLTVADSATYYCKAVFMPQHWHPAAHWYDGAGTVLTVNQASGAHHHHHHGAEFEQKLISEEDL

SEQ ID NO: 217 - protein, 254 aa, synthetic construct
  source 1..254: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACGA

SEQ ID NO: 218 - protein, 260 aa, synthetic construct
  source 1..260: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNQACGAHHHHHH

SEQ ID NO: 219 - protein, 264 aa, synthetic construct
  source 1..264: note = Artificial Sequence
TRVDQSPSSLSASVGDRVTITCVLTDTSYGLYSTSWFRKNPGTTDWERMSIGGRYVESVNKGAKSFTLTISSLQPEDFATYYCKAREARHPWLRQWYDGAGTKVEIKGGGGSGGGGSGGGGSGGGGSGGGGSTRVDQSPSSLSASVGDRVTITCVLTDTSYGLYSTSWFRKNPGTTDWERMSIGGRYVESVNKGAKSFTLTISSLQPEDFATYYCKAREARHPWLRQWYDGAGTKVEIKQASGAHHHHHHGAEFEQKLISEEDL

SEQ ID NO: 220 - protein, 264 aa, synthetic construct
  source 1..264: note = Artificial Sequence
TRVDQSPSSLSASVGDRVTITCVLTDTSYGLYSTYWYRKNPGSSNKEQISISGRYSESVNKGTKSFTLTISSLQPEDSATYYCRAREARHPWLRQWYDGAGTKVEIKGGGGSGGGGSGGGGSGGGGSGGGGSTRVDQSPSSLSASVGDRVTITCVLTDTSYGLYSTYWYRKNPGSSNKEQISISGRYSESVNKGTKSFTLTISSLQPEDSATYYCRAREARHPWLRQWYDGAGTKVEIKQASGAHHHHHHGAEFEQKLISEEDL

SEQ ID NO: 221 - protein, 264 aa, synthetic construct
  source 1..264: note = Artificial Sequence
TRVDQSPSSLSASVGDRVTITCVLTDTSYGLYSTYWYRKNPGTTDWERMSIGGRYSESVNKGAKSFTLTISSLQPEDSATYYCRAREARHPWLRQWYDGAGTKVEIKGGGGSGGGGSGGGGSGGGGSGGGGSTRVDQSPSSLSASVGDRVTITCVLTDTSYGLYSTYWYRKNPGTTDWERMSIGGRYSESVNKGAKSFTLTISSLQPEDSATYYCRAREARHPWLRQWYDGAGTKVEIKQASGAHHHHHHGAEFEQKLISEEDL

SEQ ID NO: 222 - protein, 274 aa, synthetic construct
  source 1..274: note = Artificial Sequence
ASVNQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIKGGGGSGGGGSGGGGSGGGGSGGGGSASVNQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIKQASGAHHHHHHGAEFEQKLISEEDL

SEQ ID NO: 223 - protein, 274 aa, synthetic construct
  source 1..274: note = Artificial Sequence
TRVDQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIKGGGGSGGGGSGGGGSGGGGSGGGGSTRVDQSPSSLSASVGDRVTITCVLTDTSYGLYSTSWFRKNPGTTDWERMSIGGRYSESVNKGAKSFTLTISSLQPEDSATYYCKAQSGMAISTGSGHGYNWYDGAGTKVEIKQASGAHHHHHHGAEFEQKLISEEDL

SEQ ID NO: 224 - protein, 274 aa, synthetic construct
  source 1..274: note = Artificial Sequence
ASVNQSPSSASASVGDRLTITCVLTDTSYGLYSTSWFRKNPGTTDWERMSIGGRYSESVNKGAKSFTLTISSLQPEDSATYYCKAQSGMAISTGSGHGYNWYDGAGTKLEVKGGGGSGGGGSGGGGSGGGGSGGGGSASVNQSPSSASASVGDRLTITCVLTDTSYGLYSTSWFRKNPGTTDWERMSIGGRYSESVNKGAKSFTLTISSLQPEDSATYYCKAQSGMAISTGSGHGYNWYDGAGTKLEVKQASGAHHHHHHGAEFEQKLISEEDL

SEQ ID NO: 225 - protein, 268 aa, synthetic construct
  source 1..268: note = Artificial Sequence
TRVDQSPSSLSASVGDRVTITCVLTGANYGLASTYWYRKNPGSSNQERISISGRYSESVNKRTMSFTLTISSLQPEDSATYYCRAYPWGAGAPWLVQWYDGAGTKVEIKGGGGSGGGGSGGGGSGGGGSGGGGSTRVDQSPSSLSASVGDRVTITCVLTGANYGLASTYWYRKNPGSSNQERISISGRYSESVNKRTMSFTLTISSLQPEDSATYYCRAYPWGAGAPWLVQWYDGAGTKVEIKQASGAHHHHHHGAEFEQKLISEEDL

SEQ ID NO: 226 - protein, 268 aa, synthetic construct
  source 1..268: note = Artificial Sequence
TRVDQSPSSLSASVGDRVTITCVLTGANYGLASTYWYRKNPGSSNKEQISISGRYSESVNKGTKSFTLTISSLQPEDSATYYCRAYPWGAGAPWLVQWYDGAGTKVEIKGGGGSGGGGSGGGGSGGGGSGGGGSTRVDQSPSSLSASVGDRVTITCVLTGANYGLASTYWYRKNPGSSNKEQISISGRYSESVNKGTKSFTLTISSLQPEDSATYYCRAYPWGAGAPWLVQWYDGAGTKVEIKQASGAHHHHHHGAEFEQKLISEEDL

SEQ ID NO: 227 - protein, 357 aa, synthetic construct
  source 1..357: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 228 - protein, 355 aa, synthetic construct
  source 1..355: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 229 - protein, 356 aa, synthetic construct
  source 1..356: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 356 aa, synthetic construct
  source 1..356: note = Artificial Sequence
TRVDQSPSSLSASVGDRVTITCVLTGANYGLASTYWYRKNPGSSNKEQISISGRYSESVNKGTKSFTLTISSLQPEDSATYYCRAYPWGAGAPWLVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 356 aa, synthetic construct
  source 1..356: note = Artificial Sequence
TRVDQSPSSLSASVGDRVTITCVLTGANYGLASTYWYRKNPGSSNQERISISGRYSESVNKRTMSFTLTISSLQPEDSATYYCRAYPWGAGAPWLVQWYDGAGTKVEIKGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 356 aa, synthetic construct
  source 1..356: note = Artificial Sequence
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 233 - protein, 354 aa, synthetic construct
  source 1..354: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 359 aa, synthetic construct
  source 1..359: note = Artificial Sequence
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSTRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVN

SEQ ID NO: 235 - protein, 354 aa, synthetic construct
  source 1..354: note = Artificial Sequence
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 236 - protein, 359 aa, synthetic construct
  source 1..359: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 364 aa, synthetic construct
  source 1..364: note = Artificial Sequence
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVN

SEQ ID NO: 238 - protein, 496 aa, synthetic construct
  source 1..496: note = Artificial Sequence
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 501 aa, synthetic construct
  source 1..501: note = Artificial Sequence
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVNGGGGSGGGGSGGGGSGGGGSGGGGSASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVN

SEQ ID NO: 240 - protein, 359 aa, synthetic construct
  source 1..359: note = Artificial Sequence
AKVDQTPRTATKETGESLTINCVLTDTSYGLYSTSWFRKNPGTTDWERMSIGGRYVESVNKRAKSFSLRIKDLTVADSATYYCKAQSGMAIDIGSGHGYNWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 353 aa, synthetic construct
  source 1..353: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 242 - protein, 353 aa, synthetic construct
  source 1..353: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 243 - protein, 376 aa, synthetic construct
  source 1..376: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEFC

SEQ ID NO: 244 - protein, 616 aa, synthetic construct
  source 1..616: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 363 aa, synthetic construct
  source 1..363: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 377 aa, synthetic construct
  source 1..377: note = Artificial Sequence
EEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 322 aa, synthetic construct
  source 1..322: note = Artificial Sequence
KCYNSTGVDYRGTVSVTKSGRQCQPWNSQYPHTHTFTALRFPELNGGHSYCRNPGNQKEAPWCFTLDENFKSDLCDIPACGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 512 aa, synthetic construct
  source 1..512: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 469 aa, synthetic construct
  source 1..469: note = Artificial Sequence
EEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 611 aa, synthetic construct
  source 1..611: note = Artificial Sequence
EVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 616 aa, synthetic construct
  source 1..616: note = Artificial Sequence
QETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 615 aa, synthetic construct
  source 1..615: note = Artificial Sequence
QETELSVSAELVPTSSWNTSSEIDKDSYLTLDEPMNNITTSLGQTAELHCKVSGNPPPNIRWFKNDAPVVQEPRRISFRATNYGSRLRIRNLDTTDTGYFQCVATSGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCHTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLVDKSRWQQGNVFSCSVMHEALHNHYTQKSLSLSPGK

SEQ ID NO: 253 - protein, 382 aa, synthetic construct
  source 1..382: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEHHHHHHHH

SEQ ID NO: 254 - protein, 380 aa, synthetic construct
  source 1..380: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEHHHHHH

SEQ ID NO: 255 - protein, 126 aa, synthetic construct
  source 1..126: note = Artificial Sequence
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFHHHHH

SEQ ID NO: 256 - protein, 382 aa, synthetic construct
  source 1..382: note = Artificial Sequence
QETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEHHHHHHHH

SEQ ID NO: 257 - protein, 361 aa, synthetic construct; identical to SEQ ID NO: 83
  source 1..361: note = Artificial Sequence

SEQ ID NO: 258 - protein, 371 aa, synthetic construct; identical to SEQ ID NO: 84
  source 1..371: note = Artificial Sequence

SEQ ID NO: 259 - protein, 359 aa, synthetic construct; identical to SEQ ID NO: 85
  source 1..359: note = Artificial Sequence

SEQ ID NO: 260 - protein, 369 aa, synthetic construct; identical to SEQ ID NO: 86
  source 1..369: note = Artificial Sequence

SEQ ID NO: 261 - protein, 491 aa, synthetic construct; identical to SEQ ID NO: 87
  source 1..491: note = Artificial Sequence

SEQ ID NO: 262 - protein, 501 aa, synthetic construct; identical to SEQ ID NO: 88
  source 1..501: note = Artificial Sequence

SEQ ID NO: 263 - protein, 359 aa, synthetic construct
  source 1..359: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 264 - protein, 369 aa, synthetic construct
  source 1..369: note = Artificial Sequence
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVNGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSHHHHHH

SEQ ID NO: 265 - protein, 480 aa, synthetic construct; identical to SEQ ID NO: 89
  source 1..480: note = Artificial Sequence

SEQ ID NO: 266 - protein, 490 aa, synthetic construct; identical to SEQ ID NO: 90
  source 1..490: note = Artificial Sequence

SEQ ID NO: 267 - protein, 483 aa, synthetic construct; identical to SEQ ID NO: 91
  source 1..483: note = Artificial Sequence

SEQ ID NO: 268 - protein, 493 aa, synthetic construct; identical to SEQ ID NO: 92
  source 1..493: note = Artificial Sequence

SEQ ID NO: 269 - protein, 496 aa, synthetic construct; identical to SEQ ID NO: 93
  source 1..496: note = Artificial Sequence

SEQ ID NO: 270 - protein, 506 aa, synthetic construct; identical to SEQ ID NO: 94
  source 1..506: note = Artificial Sequence